(19) United States
(12) Patent Application Publication
(10) Pub. No.: US 2025/0329414 A1
(43) Pub. Date: Oct. 23, 2025

(54) Gene Expression-Based Molecular Biomarker To Identify Lung Transplant Recipients With Chronic Lung Allograft Dysfunction

(71) Applicants: The United States Government As Represented By The Department of Veterans Affairs, Washington, DC (US); University of Pittsburgh, Pittsburgh, PA (US); The Regents of the University of California, Oakland, CA (US)

(72) Inventors: John Greenland, Washington, DC (US); John McDyer, Pittsburgh, PA (US); Charles Regis Langelier, Oakland, CA (US)

(21) Appl. No.: 19/061,693

(22) Filed: Feb. 24, 2025

Related U.S. Application Data
(60) Provisional application No. 63/557,290, filed on Feb. 23, 2024.
(60) Provisional application No. 63/563,811, filed on Mar. 11, 2024.
(60) Provisional application No. 63/674,601, filed on Jul. 23, 2024.

(51) Int. Cl. G16B 25/10; G16B 40/20; G16H 50/20

(52) U.S. Cl. CPC G16B 25/10; G16B 40/20; G16H 50/20

(57) ABSTRACT

Chronic Lung Allograft Dysfunction (CLAD) is characterized by a progressive and irreversible decline in lung function affecting half of lung transplant recipients within five years and is the major cause of death contributing to a low median post-transplant survival. Disclosed herein is an optimized airway inflammation gene set (AI2) and AI2 score, as well as RS1, RS2, and RS3 subscores, for use in methods of treating or preventing CLAD, methods of treating or preventing graft failure, methods of treating or preventing transplant rejection, methods of treating or preventing small airway fibrosis, methods of treating or preventing antibody mediated rejection (AMR) and methods of reducing mortality associated with graft failure. Also disclosed are methods of method of detecting or diagnosing CLAD and methods of predicting transplant rejection.

### CROSS-REFERENCE TO RELATED APPLICATIONS

This application claims the benefit of U.S. Provisional Patent Application No. 63/557,290, filed Feb. 23, 2024, U.S. Provisional Patent Application No. 63/563,811, filed Mar. 11, 2024, and U.S. Provisional Patent Application No. 63/674,601, filed Jul. 23, 2024, each of which is incorporated by reference herein in its entirety.

### STATEMENT REGARDING FEDERALLY SPONSORED RESEARCH

This invention was made with government support under RO1 HL161048 awarded by the National Institutes of Health. The government has certain rights in this invention.

Research for this invention was supported by awards from the Cystic Fibrosis Foundation.

### BACKGROUND

Chronic Lung Allograft Dysfunction (CLAD) is characterized by a progressive and potentially irreversible decline in lung function (1) affecting half of lung transplant recipients within five years (2). CLAD erodes quality of life benefits from transplant and is the major cause of death contributing to a low median post-transplant survival of 6-7 years (3).

Lymphocytic inflammation in large (bronchitis) and small (bronchiolitis) airways has been linked to future development of CLAD but is a relatively rare finding (4, 5). While obliterative bronchiolitis is a small airways disease, small airways are not prominently sampled on transbronchial biopsies. The use of an airway cytology brush can substantially increase sampled airway surface area with the potential for reduced risk of significant bleeding or pneumothorax when compared to transbronchial biopsy (6, 7). It was previously found that a metagene, or sum of normalized counts within a gene set, of lymphocytic bronchiolitis-associated transcripts was increased in small airway brushes from participants with CLAD as compared to airways from recipients with stable lung function (8). Separately, differential expression analysis of the CLAD airway transcriptome identified a Type-1 immune activation signature in a University of Pittsburgh cohort (9). However, it is unknown when during CLAD development that these airway inflammation signatures become apparent. A 20% decline in one-second forced expiratory volume (FEV1) reflects a substantial loss of bronchiolar tissue, so detection of CLAD prior to this threshold may identify an optimal time period for therapeutic interventions (10).

### BRIEF SUMMARY

Disclosed herein is an optimized airway inflammation gene set (AI2) for the classification of CLAD cases versus controls and prediction of subsequent graft failure or death, as well as methods of diagnosing and treating CLAD in patients by detecting or identifying subjects with increased AI2 score compared to a reference population.

Disclosed are methods of treating or preventing chronic lung allograft dysfunction (CLAD) in a subject having or at risk of developing CLAD comprising: administering a CLAD therapeutic to the subject identified in need thereof, wherein the subject was identified as being in need thereof by determining that the subject has an Airway Inflammation 2 (AI2) metagene score from a sample obtained from the subject which is higher than a reference AI2 metagene score obtained from a reference population.

Disclosed are methods of treating or preventing graft failure in a subject having or at risk of developing graft failure comprising: administering a CLAD therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

Disclosed are methods of treating or preventing transplant rejection in a subject having or at risk of developing transplant rejection comprising: administering a CLAD therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

Disclosed are methods of treating or preventing small airway fibrosis in a subject having or at risk of developing small airway fibrosis comprising: administering a CLAD therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

Disclosed are methods of treating or preventing antibody mediated rejection (AMR) in a subject having or at risk of developing AMR comprising: administering an AMR therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

Disclosed are methods of reducing mortality associated with graft failure in a subject at risk of mortality associated with graft failure comprising: administering a CLAD therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

Disclosed are methods of method of detecting or diagnosing CLAD in a subject at risk of developing CLAD comprising a) determining an AI2 metagene score from a sample of cells from the airway of the subject; and b) comparing the AI2 metagene score to an AI2 metagene score obtained from a reference population; wherein the AI2 metagene score is higher than the AI2 metagene score obtained from the reference population detects or diagnoses CLAD in the subject.

Disclosed are methods of predicting transplant rejection in a subject at risk of developing transplant rejection comprising: a) determining an AI2 metagene score from a sample of cells from the airway of the subject; and b) comparing the AI2 metagene score to an AI2 metagene score obtained from a reference population; wherein when the AI2 metagene score is higher than the AI2 metagene score obtained from the reference population indicates the subject is at a higher risk for developing transplant rejection.

Disclosed are methods of identifying an effective therapeutic for treatment of CLAD in a subject comprising determining an RS1 score from a sample from a subject, wherein when the RS1 score from the sample obtained from the subject is higher than an RS1 score obtained from a reference population, thereby identifying an effective therapeutic for treatment of CLAD in the subject, wherein the effective therapeutic is cyclosporine, tacrolimus, baricitinib and/or belatacept.

Disclosed are methods of identifying an effective therapeutic for treatment of CLAD in a subject comprising determining an RS2 score from a sample from a subject, wherein when the RS2 score from the sample obtained from the subject is higher than an RS2 score obtained from a reference population, thereby identifying an effective therapeutic for treatment of CLAD in the subject, wherein the effective therapeutic is azithromycin, tocilizumab and/or calcineurin inhibitors.

Disclosed are methods of identifying an effective therapeutic for treatment of CLAD in a subject comprising determining an RS3 score from a sample from a subject, wherein when the RS3 score from the sample obtained from the subject is higher than an RS3 score obtained from a reference population, thereby identifying an effective therapeutic for treatment of CLAD in the subject, wherein the effective therapeutic is ECP, TLI, thymoglobulin, anti-CD94 monoclonal antibody and/or belumosudil.

Disclosed are methods of screening for a candidate therapeutic that can treat CLAD in a subject having or at risk of developing CLAD comprising a) determining a first AI2 metagene score from a sample of cells from the subject having or at risk of developing CLAD; b) adding a candidate therapeutic to the sample of cells; c) determining a second AI2 metagene score from the sample after incubating the sample of cells with the candidate therapeutic; and d) determining the candidate therapeutic treats CLAD when the second AI2 metagene score is lower than the first AI2 metagene score.

Disclosed are methods of screening whether a subject having CLAD is responsive to a therapeutic comprising a) determining a first AI2 metagene score from a first sample of cells from the subject having CLAD; b) administering the therapeutic to the subject; c) determining a second AI2 metagene score from a second sample of cells taken after administering the therapeutic to the subject; and d) determining the subject is responsive to the therapeutic when the second AI2 metagene score is lower than the first AI2 metagene score.

Disclosed are methods of identifying a subject for a clinical study for a CLAD therapeutic, comprising a) determining an AI2 metagene score from a sample of cells from the subject; wherein an AI2 metagene score above 0 indicates the subject is appropriate for the clinical study for a CLAD therapeutic.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a study enrollment flow (CONSORT) diagram.

FIGS. 2A-2B show candidate gene scores distinguish participants with CLAD from control participants in airway brushes before and after CLAD onset. (FIG. 2A) FEV1 as a smoothed function of time from airway brush is shown for the before, early-CLAD, and late-CLAD cohorts, with cases in red and controls in blue. (FIG. 2B) Metagene scores were calculated for 4 candidate gene scores in CLAD cases and controls for the three time groups. Unadjusted Mann-Whitney test p-values are shown. * denotes P<0.05; ** denotes P<0.01, and *** denotes P<0.001.

FIGS. 3A-3E show derivation of the Airway Inflammation 2 (AI2) metagene set. (FIG. 3A) The union of the Common Rejection Module, UCSF Lymphocytic Bronchitis, the Renal Rejection versus Everything Else, and Pitt CLAD score yielded 81 candidate genes. Each gene was assessed for association with CLAD and time to graft failure, and 49 genes were excluded because of a P-value >0.05 in at least one model. (FIG. 3B) A metagene score, or sum of normalized gene counts, from the 32 AI2 genes distinguished CLAD from controls in all three timepoints from the derivation cohort. P-values were determined by Mann-Whitney test. (FIG. 3C) The AI2 score stability over time was assessed across time points, with Pearson correlation coefficient shown. Lines correspond to individual subjects. Parentheses indicate 95% confidence intervals. (FIG. 3D) The AI2 metagene score is shown stratified by CLAD stage at the time of brush in cases and control brushes from the derivation cohort. CLAD stage 0 cases correspond to the before-CLAD group, whereas stages 1, 2, and 3 derive from the early and late CLAD groups. Differences versus control group were determined by GEE-adjusted regression to account for repeat brushes on the same subject (FIG. 3E) AI2 metagene scores are shown in cases and controls stratified by the presence of viral or bacterial pathogens on clinical BAL microbial testing. Differences between groups were determined by GEE-adjusted regression. * denotes P<0.05; ** denotes P<0.01, *** denotes P<0.001, and **** denotes P<0.0001.

FIGS. 4A-4B: AI2 subcomponent analysis. (FIG. 4A) Hierarchical clustering of cross-correlation within the 32 AI2 genes identified 3 separate clusters of co-expressed genes, which were identified as associated with antigen presentation, cytokine signaling, and lymphocyte activation, by Gene Ontology enrichment analysis. (FIG. 4B) A metagene score for each cluster was compared in cases and controls in before CLAD, early CLAD, and late CLAD time points of the derivation cohort. Unadjusted Mann-Whitney test p-values are shown from which * denotes P<0.05; ** is P<0.01, and *** is P<0.001.

FIG. 5 shows unbiased machine learning classifier performance in the three derivation timepoints. Receiver operating curves (ROC) are shown for leave one out cross-validated extreme gradient boosting models derived from all observed genes in the before, early and late CLAD time points of the derivation cohort. The area under the ROC curve (AUC) for before CLAD was 0.61 (95% CI 0.45-0.61), early CLAD AUC was 0.82 (95% CI 0.71-0.82, P=0.03 versus before CLAD), and late CLAD AUC was 0.83 (95% CI 0.67-0.83, P=0.06 versus before CLAD).

FIGS. 6A-6F show associations with CLAD, subsequent graft failure, and survival in derivation and validation cohorts. (FIG. 6A) Receiver Operating Curve (ROC) analysis for AI2 scores as a predictor of CLAD case versus control assignments both in (FIG. 6A) UCSF derivation and (FIG. 6B) Pitt validation cohorts. Area under ROC curve (AUC) for before CLAD was 0.79 (95% CI 0.66-0.91), early CLAD AUC was 0.89 (95% CI 0.80-0.97), and late CLAD AUC was 0.74 (95% CI 0.60-0.88). AUC for CLAD was 0.78 (95% CI 0.68-0.88) in the validation cohort. (FIG. 6C-FIG. 6D) Time to event analyses for graft failure, defined as death with CLAD or retransplantation, as a function of years from airway brush, stratified by median AI2 score are shown for the (FIG. 6C) derivation cohort and (FIG. 6D) validation cohort. (FIG. 6E-FIG. 6F) Kaplan-Meier curves for all-cause mortality showing survival versus time from airway brush stratified by tertile of AI2 score as a function of time to death from the date of airway brush stratified by AI2 score in (FIG. 6E) derivation cohort and (FIG. 6F) validation cohort. Confidence intervals in A-B reflect DeLong method. P-values in FIG. 6C-FIG. 6F are based on log-rank test.

FIGS. 7A-7F. Study design and FEV₁ trajectories across ALAD strata. FIG. 7A) Participants were screened based on FEV1 and charts reviewed for documentation of concern for ALAD. Some participants targeted for participation could not be enrolled based on safety concerns or technical issues. FIG. 7B) Controls were collected based on FEV1 stability, with some exclusions for technical issues. FIGS. 7C-7F) FEV1 trajectories before and after brush collection (performed at time 0). Each line represents a different individual. FIG. 7C) Controls (n=8). FIGS. 7D-7F) Individuals with ALAD, whose FIG. 7D) FEV1 recovered to pre-ALAD levels (n=4), FIG. 7E) FEV1 persisted at the lower-level post ALAD (n=5), and FIG. 7F) FEV1 further declined following ALAD episode (n=3).

FIGS. 8A-8G. Cell proportions in small airways differ as a function of ALAD outcome. FIG. 8A) Heatmap of cell markers used for cluster identification. FIG. 8B) UMAP of 68,140 high-quality cells, color-coded by cell subtype (labels under UMAP, categorized by lineage). FIG. 8C) UMAP of cells color-coded to their ALAD outcome grouping. Cells from each group were down sampled to 9000 for visualization purposes. FIG. 8D) Multidimensional scaling (MDS) analysis assessed robust Aitchison distance between cell counts for each cluster. Individuals are dots, color-coded by their ALAD grouping. Larger dots are the average per grouping. P-value reflects PERMANOVA of cell count distances compared with ALAD outcome grouping. FIG. 8E) Correlation coefficients of individual cell subset frequencies with MDS component 1 (x-axis) and MDS component 2 (y-axis). All coefficients were calculated using Spearman correlation. F-G) Comparison of cell proportions across ALAD outcome groups. FIG. 8F) epithelial cell subsets (% of EPCAM+ epithelial cells); FIG. 8G) non-epithelial subsets (% of EPCAM-non-epithelial subsets, excluding neutrophils). Spearman correlation coefficients and P-values are shown for the association between cell frequency and ALAD outcome grouping.

FIGS. 9A-9D. A CLAD-associated gene set is increased in epithelial subsets from recipients with persistent and progressive ALAD. FIG. 9A) Feature plot of AI2 score on UMAP of cells from control samples. FIG. 9B) Ridge plot of AI2 score distributions per cell subset on control samples. FIG. 9C) Comparison of AI2 score across ALAD outcome groups for cell subsets. P-values reflect mixed effects modeling of AI2 score as a function of worsening ALAD outcome, with participant ID as a random effect. FIG. 9D) Heatmap of AI2 score genes versus cell type and ALAD outcome groups. AI2 subcomponents to which the genes belong are noted on the left of the genes. Yellow indicates greater expression. Cells were downsampled at the inverse of cell frequency to 10,000 cells for more even representation.

FIGS. 10A-10D. Shift in transcriptomic profiles of basal cells and lymphocytes with severe ALAD. FIG. 10A) Gene set enrichment analysis was performed using Reactome pathways across cell types. Yellow indicates a positive association with worst ALAD outcome, while purple indicates enrichment in controls. Normalized enrichment score (NES) represents enrichment of gene set per unit increase in ALAD outcome severity group. FIG. 10B) Per cell type, percentage of all genes detected in the cell type that were differentially expressed (FDR <0.1) as a function of ALAD outcome grouping are shown. Cell populations with no significant GSEA hits were not included. FIGS. 10B-10C) Volcano plots showing genes with increased (yellow) or decreased (blue) expression with worst ALAD outcome among FIG. 10C) basal cells or FIG. 10D) lymphocytes. Results show the log fold change in gene expression (x-axis) per unit increase in ALAD outcome severity group, with FDR-adjusted log p-values on the y-axis.

FIGS. 11A-11F. CD8+ T cell activation and loss of alveolar macrophages in non-resolving ALAD. FIGS. 11A-11B) UMAP representation of lymphocyte subset clusters, color-coded by FIG. 11A) cluster identification or FIG. 11B) ALAD outcome groupings. FIG. 11C) Proportion of lymphocyte subsets on total lymphocytes, compared across ALAD outcome groupings. FIGS. 11D-11E) UMAP representation of myeloid subsets, color-coded by FIG. 11D) cluster identification or FIG. 11E) ALAD outcome groupings. F) Proportion of myeloid subsets on total myeloid, compared across ALAD outcome. Pearson correlation coefficients and P-values are shown for the association between cell frequency and ALAD outcome groupings.

FIGS. 12A-12C Single cell RNA sequencing analysis of the AI2 score identifies relevant cell types to enhance classification accuracy. FIG. 12A) Airway brushes were prospectively collected and single cell sequencing libraries for 16 CLAD cases and 17 controls were generated. Cell types were identified with based on Leuven clustering and comparison of cluster-driving genes with reference sets. The AI2 module was expressed in immune cells and epithelial cell subsets. FIG. 12B) A module score was calculated for the genes in the AI2 gene set as well as for cell type identifier reference genes (SCGB3A1, MS4A8, KRT5, and CALM1). Subtracting reference genes from the AI2 gene set improved differentiation between cases and controls (blue versus red). FIG. 12C) Random forest machine learning models were built using genes of the AI2 gene set with raw count values from before (N=51) and after CLAD onset (N=99). Adding cell type identifiers boosted the performance of machine learning models to approximate that of the original AI2 score.

FIG. 13 illustrates a flowchart for a lung allograft dysfunction diagnosis system 1300.

FIG. 14 illustrates a flowchart of a gene expression data processing method 1400.

FIG. 15 is a block diagram illustrating an exemplary operating environment for performing the disclosed methods.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an exosome” includes a plurality of such exosomes, reference to “the exosome” is a reference to one or more exosomes and equivalents thereof known to those skilled in the art, and so forth.

By a “therapeutically effective amount” of a composition as provided herein is meant a sufficient amount of the composition to provide the desired therapeutic effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “therapeutically effective amount.” However, an appropriate “therapeutically effective amount” may be determined by one of ordinary skill in the art using only routine experimentation.

The term “therapeutic” refers to a composition that treats a disease. The term “CLAD therapeutic” as disclosed herein are compositions that treat Chronic Lung Allograft Dysfunction (CLAD). Examples of CLAD therapeutics include, but are not limited to, macrolide antibiotic azithromycin, calcineurin inhibitors such as cyclosporine and tacrolimus, aerosolized cyclosporine, fundoplication for gastroesophageal reflux, montelukast, extracorporeal photopheresis (ECP), cytolytic anti-lymphocyte therapies, anti-human thymocyte globulin such as thymoglobulin, total lymphoid irradiation (TLI), pirfenidone, mTor inhibitors such as everolimus, sirolimus/rapamycin and inhaled rapamycin, macitentan, prednisone, inhibitors of the JAK/STAT pathway such as baricitinib, anti-CD94 monoclonal antibodies, such as DR-01, aztreonam lysine inhalation, tocilizumab, mesenchymal stem cells, regadenoson, Rho-kinase inhibitors such as belumosudil, immunoglobulin and/or co-stimulatory blockade therapeutics such as belatacept.

Calcineurin inhibitors include, but are not limited to, cyclosporine and tacrolimus. mTor inhibitors include, but are not limited to, rapamycin (sirolimus) and everolimus. JAK/STAT inhibitors include, but are not limited to, baricitinib. Co-stimulatory blockade therapeutics include, but are not limited to, belacitinib. Anti-CD94 monoclonal antibodies include, but are not limited to, DR-01. Rho-kinase inhibitors include, but are not limited to, belumosudil. Anti-human thymocyte globulins include, but are not limited to, thymoglobulin.

By “AMR therapeutic” as disclosed herein are compositions that treat antibody mediated rejection (AMR). Examples of AMR therapeutics include, but are not limited to, rituximab, intravenous immune globulin (IVIG), plasmapheresis, anti-thymocyte globulin, daratumumab, bortezomib, carfilzomib, tocilizumab, belimumab, C1 esterase inhibitor, plerixafor, a combination of these, or pharmaceuticals of the same classes.

By “treat” is meant to administer a therapeutic or composition of the invention to a subject, such as a human or other mammal (for example, an animal model), that has an increased susceptibility for developing CLAD, graft failure, transplant rejection, small airway fibrosis or antibody mediated rejection (AMR), or that has CLAD, graft failure, transplant rejection, small airway fibrosis or AMR, in order to prevent or delay a worsening of the effects or symptoms of the disease or condition, or to partially or fully reverse the effects of the disease.

By “prevent” is meant to minimize the chance that a subject who has an increased susceptibility for developing a disease or disorder, such as CLAD, graft failure, transplant rejection, small airway fibrosis, or AMR or will end up with the disease or disorder, such as CLAD, graft failure, transplant rejection, small airway fibrosis or AMR.

The terms “patient,” “subject,” “individual,” and the like are used interchangeably herein, and refer to any animal, or cells thereof whether in vitro or in situ, amenable to the methods described herein. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered. In some aspects, the subject is a lung transplant recipient.

An “effective amount” of a compound is that amount of compound which is sufficient to provide a beneficial effect to the subject to which the compound is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a delivery vehicle is that amount sufficient to effectively bind or deliver a compound.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

By “metagene” is meant to represent a collection of genes behaving in a functionally correlated fashion within the genome.

By “metagene score” is meant a sum of normalized counts within a gene set. Metagene scores are subsequently standardized to facilitate interpretation. Neither normalization nor standardization is strictly required for these scores to be effective.

The following human genes are part of the AI2 metagene. The NCBI Gene ID Nos. for the genes in the AI2 metagenes are set out in Table 1a, Table 1b or Table 1c. Sequences and additional information about the gene can be accessed can be accessed at ncbi.nlm.nih.gov/gene.

TABLE 1a
Gene Sequences

| Gene Name | NCBI Gene ID: |
|---|---|
| MYH9 | 4627 |
| SAT1 | 6303 |
| TPM4 | 7171 |
| MDK | 4192 |
| UBD | 10537 |
| CD74 | 972 |
| HLA-A | 3105 |
| HLA-E | 3133 |
| HLA-C | 3107 |
| HLA-B | 3106 |
| IRF1 | 3659 |
| PSMB9 | 5698 |
| PSMB8 | 5696 |
| ISG20 | 3669 |
| MIDN | 90007 |
| APOL3 | 80833 |
| CXCL11 | 6373 |
| CXCL10 | 3627 |
| CXCL9 | 4283 |
| GBP4 | 115361 |
| NLRC5 | 84166 |
| IDO1 | 3620 |
| TAP1 | 6890 |
| HLA-F | 3134 |
| SERPINA3 | 12 |
| ADAMDEC1 | 27299 |
| CXCL13 | 10563 |
| INPP5D | 3635 |
| KLRD1 | 3824 |
| FCAR | 2204 |
| NKG7 | 4818 |
| ADORA2A | 135 |

TABLE 1b
Gene Sequences

| Gene Name | NCBI Gene ID: |
|---|---|
| TPM4 | 7171 |
| MDK | 4192 |
| UBD | 10537 |
| HLA-A | 3105 |
| HLA-E | 3133 |
| HLA-C | 3107 |
| HLA-B | 3106 |
| PSMB9 | 5698 |
| PSMB8 | 5696 |
| CXCL10 | 3627 |
| CXCL9 | 4283 |
| GBP4 | 115361 |
| NLRC5 | 84166 |
| IDO1 | 3620 |
| TAP1 | 6890 |
| CXCL13 | 10563 |
| KLRD1 | 3824 |

TABLE 1c
Gene Sequences

| Gene Name | NCBI Gene ID: |
|---|---|
| SAT1 | 6303 |
| TPM4 | 7171 |
| MDK | 4192 |
| HLA-A | 3105 |
| HLA-E | 3133 |
| IRF1 | 3659 |
| PSMB9 | 5698 |
| CXCL11 | 6373 |
| GBP4 | 115361 |
| NLRC5 | 84166 |
| IDO1 | 3620 |
| TAP1 | 6890 |
| HLA-F | 3134 |
| ADAMDEC1 | 27299 |
| CXCL13 | 10563 |
| KLRD1 | 3824 |
| NKG7 | 4818 |

By CLAD is meant the clinical manifestations of pathological processes in the airway and parenchymal compartments of the lung allograft that lead to a significant and persistent deterioration of lung function. As used herein, CLAD refers to persistent and irreversible decline in forced expiratory volume in 1 second (FEV1) of at least 20% compared to the mean of the two best post-operative values at least 3 weeks apart.

Antibody mediated rejection (AMR) can be another cause of CLAD and loss of graft function. Criterial for AMR are defined in the Antibody-mediated rejection of the lung: A consensus report of the International Society for Heart and Lung Transplantation—The Journal of Heart and Lung Transplantation (jhltonline.org)). Antibody-mediated rejection (AMR) in lung transplant is diagnosed based on hallmark features of graft dysfunction, complement deposition, donor specific antibodies, and consistent histopathology. AMR can drive rapid graft failure and mortality, but often is more indolent, and challenging to distinguish from other forms of graft dysfunction. The AI2 metagene score is useful in identifying individuals with AMR who are at increased risk for CLAD or death.

CLAD also refers to the two subtypes of CLAD, bronchiolitis obliterans syndrome (BOS) and restrictive allograft syndrome (RAS), which are described in ISHLT consensus guidelines. (Chronic lung allograft dysfunction: Definition and update of restrictive allograft syndrome—A consensus report from the Pulmonary Council of the ISHLT—The Journal of Heart and Lung Transplantation (jhltonline.org)).

CLAD also refers to both acute lung allograft dysfunction (ALAD) and suspected CLAD. Definitions of ALAD and suspected CLAD have been proposed and are currently being formally defined through ISHLT (a new classification system for chronic lung allograft dysfunction—ScienceDirect). ALAD and suspected CLAD precede the diagnosis of CLAD and are both detected by the AI2 metagene score described herein. The AI2 metagene score is useful in determining individuals with ALAD and suspected CLAD who will go on to develop CLAD. The term “ALAD” also refers to a patient having a 10% decrease in forced expiratory volume (FEV1) compared to previous FEV1 measurements from the same patient, optionally compared to baseline FEV1 measurements after lung transplantation.

By “normalize” is meant transforming counts of a specific gene to conform to a roughly normal distribution. Normalizing can be done by any method known in the art, including but not limited to variance stabilizing transformation (VST) and regularized log (rlog) transformation.

By “reference population” is meant a population of lung transplant recipients undergoing evaluation post-transplant and including an even number of subjects with CLAD and subjects having stable lung function.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

### 1. Methods of Treating

Disclosed are methods of treating or preventing chronic lung allograft dysfunction (CLAD) in a subject having or at risk of developing CLAD comprising: administering a CLAD therapeutic to the subject identified in need thereof, wherein the subject was identified as being in need thereof by determining that the subject has an Airway Inflammation 2 (AI2) metagene score from a sample obtained from the subject which is higher than a control AI2 metagene score obtained from a reference population.

In some aspects of the methods of treating or preventing CLAD in a subject having or at risk of developing CLAD, the AI2 metagene score comprises one or more of an RS1 subscore, RS2 subscore, or RS3 subscore.

In some aspects of the methods of treating or preventing CLAD in a subject having or at risk of developing CLAD, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of treating or preventing CLAD in a subject having or at risk of developing CLAD, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of treating or preventing CLAD in a subject having or at risk of developing CLAD, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of treating or preventing CLAD in a subject having or at risk of developing CLAD, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of treating or preventing CLAD in a subject having or at risk of developing CLAD, the methods further comprise determining an RS1 subscore, RS2 subscore and/or RS3 subscore.

In some aspects of the methods of treating or preventing CLAD in a subject having or at risk of developing CLAD, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

In some aspects of the methods of treating or preventing CLAD in a subject having or at risk of developing CLAD, the RS1 subscore comprises expression data of an RS1 gene cluster comprising the genes PSMB9, HLA-A, SAT1, TPM4, MDK, HLA-E, and IRF1.

In some aspects of the methods of treating or preventing CLAD in a subject having or at risk of developing CLAD, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F.

In some aspects of the methods of treating or preventing CLAD in a subject having or at risk of developing CLAD, the RS2 subscore comprises expression data of an RS2 gene cluster comprising the genes TAP1, CXCL11, GBP4, HLA-F, IDO1, and NLRC5.

In some aspects of the methods of treating or preventing CLAD in a subject having or at risk of developing CLAD, the RS3 subscore comprises expression data of an RS3 gene cluster comprising the genes SERPINA3, ADAMDEC1, CXCL13, INPP5D, KLRD1, FCAR, NKG7, and ADORA2A.

In some aspects of the methods of treating or preventing CLAD in a subject having or at risk of developing CLAD, the RS3 subscore comprises expression data of an RS3 gene cluster comprising the genes NKG7, KLRD1, ADAMDEC1, and CXCL13.

In some aspects of the methods of treating or preventing CLAD in a subject having or at risk of developing CLAD, the RS1 subscore is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of treating or preventing CLAD in a subject having or at risk of developing CLAD, the RS2 subscore is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of treating or preventing CLAD in a subject having or at risk of developing CLAD, the RS3 subscore is determined by a) normalizing the number of copies of each gene in the RS3 gene cluster by variance stabilizing transformation to obtain a subject's normalized gene count for each gene in the RS3 gene cluster; b) calculating a subject's total normalized gene count by adding the subject's normalized gene counts for each gene in the RS3 gene cluster; c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS3 subscore.

In some aspects of the methods of treating or preventing CLAD in a subject having or at risk of developing CLAD, the AI2 metagene score is greater than 0.43. In some aspects of the methods of treating or preventing CLAD in a subject having or at risk of developing CLAD, the AI2 metagene score is greater than 0. In some aspects of the methods of treating or preventing CLAD in a subject having or at risk of developing CLAD, the AI2 metagene score is between 0 and 1, 0 and 2, 0 and 3, 0 to 4 and 0 and 5. In some aspects of the methods of treating or preventing CLAD in a subject having or at risk of developing CLAD, the AI2 metagene score is between 0.43 and 3.

In some aspects of the methods of treating or preventing CLAD in a subject having or at risk of developing CLAD, the sample was obtained from small airways. In some aspects of the methods of treating or preventing CLAD in a subject having or at risk of developing CLAD, the sample was obtained by a cytology brush. In some aspects of the methods of treating or preventing CLAD in a subject having or at risk of developing CLAD, the sample was obtained by bronchoscopy.

In some aspects of the methods of treating or preventing CLAD in a subject having or at risk of developing CLAD, the subject's AI2 metagene score was determined by isolating RNA from the sample.

In some aspects of the methods of treating or preventing CLAD in a subject having or at risk of developing CLAD, the CLAD therapeutic is macrolide antibiotic azithromycin, cyclosporine, tacrolimus, fundoplication for gastroesophageal reflux, montelukast, extracorporeal photopheresis (ECP), aerosolized cyclosporine, cytolytic anti-lymphocyte therapies, thymoglobulin, total lymphoid irradiation (TLI), pirfenidone, everolimus, sirolimus, rapamycin, inhaled rapamycin, macitentan, prednisone, baricitinib, anti-CD94 monoclonal antibody, aztreonam lysine inhalation, tocilizumab, mesenchymal stem cells, regadenoson, belumosudil, immunoglobulin and/or belatacept.

In some aspects of the methods of treating or preventing CLAD in a subject having or at risk of developing CLAD, said step of administering a CLAD therapeutic to a subject identified in need thereof comprises administering a higher dose of said CLAD therapeutic than had been administered prior to treating a subject having CLAD.

In some aspects of the methods of treating or preventing CLAD in a subject having or at risk of developing CLAD, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of treating or preventing CLAD in a subject having or at risk of developing CLAD, the AI2 gene score is used as input for a random forest machine learning model. In some aspects of the methods of treating or preventing CLAD in a subject having or at risk of developing CLAD, gene expression specific to basal, club, secretory, secretory-ciliated, ciliated, ionocyte, mast, lymphocyte, and/or monocyte cells is used to normalize and improve the accuracy of a classifier.

In some aspects of the methods of treating or preventing CLAD in a subject having or at risk of developing CLAD, the epithelial subtype genes include one or more of SCGB3A1, MS4A8, KRT5, and CALM1. In some aspects of the methods of treating or preventing CLAD in a subject having or at risk of developing CLAD, the leukocyte genes include one or more of PTPRC, MARCO, and GNLY.

Disclosed are methods of treating or preventing graft failure in a subject having or at risk of developing graft failure comprising: administering a CLAD therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a control population.

In some aspects of the methods of treating or preventing graft failure in a subject having or at risk of developing graft failure, the AI2 metagene score comprises one or more of an RS1 subscore, RS2 subscore, or RS3 subscore.

In some aspects of the methods of treating or preventing graft failure in a subject having or at risk of developing graft failure, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of treating or preventing graft failure in a subject having or at risk of developing graft failure, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of treating or preventing graft failure in a subject having or at risk of developing graft failure, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of treating or preventing graft failure in a subject having or at risk of developing graft failure, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of treating or preventing graft failure in a subject having or at risk of developing graft failure, the methods further comprise determining an RS1 subscore, RS2 subscore and/or RS3 subscore.

In some aspects of the methods of treating or preventing graft failure in a subject having or at risk of developing graft failure, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

In some aspects of the methods of treating or preventing graft failure in a subject having or at risk of developing graft failure, the RS1 subscore comprises expression data of an RS1 gene cluster comprising the genes PSMB9, HLA-A, SAT1, TPM4, MDK, HLA-E, and IRF1.

In some aspects of the methods of treating or preventing graft failure in a subject having or at risk of developing graft failure, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F.

In some aspects of the methods of treating or preventing graft failure in a subject having or at risk of developing graft failure, the RS2 subscore comprises expression data of an RS2 gene cluster comprising the genes TAP1, CXCL11, GBP4, HLA-F, IDO1, and NLRC5.

In some aspects of the methods of treating or preventing graft failure in a subject having or at risk of developing graft failure, the RS3 subscore comprises expression data of an RS3 gene cluster comprising the genes SERPINA3, ADAMDEC1, CXCL13, INPP5D, KLRD1, FCAR, NKG7, and ADORA2A.

In some aspects of the methods of treating or preventing graft failure in a subject having or at risk of developing graft failure, the RS3 subscore comprises expression data of an RS3 gene cluster comprising the genes NKG7, KLRD1, ADAMDEC1, and CXCL13.

In some aspects of the methods of treating or preventing graft failure in a subject having or at risk of developing graft failure, the RS1 subscore is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of treating or preventing graft failure in a subject having or at risk of developing graft failure, the RS2 subscore is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of treating or preventing graft failure in a subject having or at risk of developing graft failure, the RS3 subscore is determined by a) normalizing the number of copies of each gene in the RS3 gene cluster by variance stabilizing transformation to obtain a subject's normalized gene count for each gene in the RS3 gene cluster; b) calculating a subject's total normalized gene count by adding the subject's normalized gene counts for each gene in the RS3 gene cluster; c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS3 subscore.

In some aspects of the methods of treating or preventing graft failure in a subject having or at risk of developing graft failure, the AI2 metagene score is greater than 0.43. In some aspects of the methods of treating or preventing graft failure in a subject having or at risk of developing graft failure, the AI2 metagene score is greater than 0. In some aspects of the methods of treating or preventing graft failure in a subject having or at risk of developing graft failure, the AI2 metagene score is between 0 and 1, 0 and 2, 0 and 3, 0 and 4 or 0 and 5. In some aspects of the methods of treating or preventing graft failure in a subject having or at risk of developing graft failure, the AI2 metagene score is between 0.43 and 3.

In some aspects of the methods of treating or preventing graft failure in a subject having or at risk of developing graft failure, the sample was obtained from small airways. In some aspects of the methods of treating or preventing graft failure in a subject having or at risk of developing graft failure, the sample was obtained by a cytology brush. In some aspects of the methods of treating or preventing graft failure in a subject having or at risk of developing graft failure, the sample was obtained by bronchoscopy.

In some aspects of the methods of treating or preventing graft failure in a subject having or at risk of developing graft failure, the subject's AI2 metagene score was determined by isolating RNA from the sample.

In some aspects of the methods of treating or preventing graft failure in a subject having or at risk of developing graft failure, the CLAD therapeutic is macrolide antibiotic azithromycin, cyclosporine, tacrolimus, fundoplication for gastroesophageal reflux, montelukast, extracorporeal photopheresis (ECP), aerosolized cyclosporine, cytolytic anti-lymphocyte therapies, thymoglobulin, total lymphoid irradiation (TLI), pirfenidone, everolimus, sirolimus, rapamycin, inhaled rapamycin, macitentan, prednisone, baricitinib, anti-CD94 monoclonal antibody, aztreonam lysine inhalation, tocilizumab, mesenchymal stem cells, regadenoson, belumosudil, immunoglobulin and/or belatacept.

In some aspects of the methods of treating or preventing graft failure in a subject having or at risk of developing graft failure, said step of administering a CLAD therapeutic to a subject identified in need thereof comprises administering a higher dose of said CLAD therapeutic than had been administered prior to treating a subject having graft failure.

In some aspects of the methods of treating or preventing graft failure in a subject having or at risk of developing graft failure, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of treating or preventing graft failure in a subject having or at risk of developing graft failure, the AI2 gene score is used as input for a random forest machine learning model. In some aspects of the methods of treating or preventing graft failure in a subject having or at risk of developing graft failure, gene expression specific to basal, club, secretory, secretory-ciliated, ciliated, ionocyte, mast, lymphocyte, and/or monocyte cells is used to normalize and improve the accuracy of a classifier.

In some aspects of the methods of treating or preventing graft failure in a subject having or at risk of developing graft failure, the epithelial subtype genes include one or more of SCGB3A1, MS4A8, KRT5, and CALM1. In some aspects of the methods of treating or preventing graft failure in a subject having or at risk of developing graft failure, the leukocyte genes include one or more of PTPRC, MARCO, and GNLY.

Disclosed are methods of treating or preventing transplant rejection in a subject having or at risk of developing transplant rejection comprising: administering a CLAD therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

In some aspects of the methods of treating or preventing transplant rejection in a subject having or at risk of developing transplant rejection, the AI2 metagene score comprises one or more of an RS1 subscore, RS2 subscore, or RS3 subscore.

In some aspects of the methods of treating or preventing transplant rejection in a subject having or at risk of developing transplant rejection, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of treating or preventing transplant rejection in a subject having or at risk of developing transplant rejection, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of treating or preventing transplant rejection in a subject having or at risk of developing transplant rejection, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of treating or preventing transplant rejection in a subject having or at risk of developing transplant rejection, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of treating or preventing transplant rejection in a subject having or at risk of developing transplant rejection, the methods further comprise determining an RS1 subscore, RS2 subscore and/or RS3 subscore.

In some aspects of the methods of treating or preventing transplant rejection in a subject having or at risk of developing transplant rejection, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

In some aspects of the methods of treating or preventing transplant rejection in a subject having or at risk of developing transplant rejection, the RS1 subscore comprises expression data of an RS1 gene cluster comprising the genes PSMB9, HLA-A, SAT1, TPM4, MDK, HLA-E, and IRF1.

In some aspects of the methods of treating or preventing transplant rejection in a subject having or at risk of developing transplant rejection, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F.

In some aspects of the methods of treating or preventing transplant rejection in a subject having or at risk of developing transplant rejection, the RS2 subscore comprises expression data of an RS2 gene cluster comprising the genes TAP1, CXCL11, GBP4, HLA-F, IDO1, and NLRC5.

In some aspects of the methods of treating or preventing transplant rejection in a subject having or at risk of developing transplant rejection, the RS3 subscore comprises expression data of an RS3 gene cluster comprising the genes SERPINA3, ADAMDEC1, CXCL13, INPP5D, KLRD1, FCAR, NKG7, and ADORA2A.

In some aspects of the methods of treating or preventing transplant rejection in a subject having or at risk of developing transplant rejection, the RS3 subscore comprises expression data of an RS3 gene cluster comprising the genes NKG7, KLRD1, ADAMDEC1, and CXCL13.

In some aspects of the methods of treating or preventing transplant rejection in a subject having or at risk of developing transplant rejection, the RS1 subscore is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of treating or preventing transplant rejection in a subject having or at risk of developing transplant rejection, the RS2 subscore is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of treating or preventing transplant rejection in a subject having or at risk of developing transplant rejection, the RS3 subscore is determined by a) normalizing the number of copies of each gene in the RS3 gene cluster by variance stabilizing transformation to obtain a subject's normalized gene count for each gene in the RS3 gene cluster; b) calculating a subject's total normalized gene count by adding the subject's normalized gene counts for each gene in the RS3 gene cluster; c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS3 subscore.

In some aspects of the methods of treating or preventing transplant rejection in a subject having or at risk of developing transplant rejection, the AI2 metagene score is greater than 0.43. In some aspects of the methods of treating or preventing transplant rejection in a subject having or at risk of developing transplant rejection, the AI2 metagene score is greater than 0. In some aspects of the methods of treating or preventing transplant rejection in a subject having or at risk of developing transplant rejection, the AI2 metagene score is between 0 and 1, 0 and 2, 0 and 3, 0 and 4 or 0 and 5. In some aspects of the methods of treating or preventing transplant rejection in a subject having or at risk of developing transplant rejection, the AI2 metagene score is between 0.43 and 3.

In some aspects of the methods of treating or preventing transplant rejection in a subject having or at risk of developing transplant rejection, the sample was obtained from small airways. In some aspects of the methods of treating or preventing transplant rejection in a subject having or at risk of developing transplant rejection, the sample was obtained by a cytology brush. In some aspects of the methods of treating or preventing transplant rejection in a subject having or at risk of developing transplant rejection, the sample was obtained by bronchoscopy.

In some aspects of the methods of treating or preventing transplant rejection in a subject having or at risk of developing transplant rejection, the subject's AI2 metagene score was determined by isolating RNA from the sample.

In some aspects of the methods of treating or preventing transplant rejection in a subject having or at risk of developing transplant rejection, the CLAD therapeutic is macrolide antibiotic azithromycin, cyclosporine, tacrolimus, fundoplication for gastroesophageal reflux, montelukast, extracorporeal photopheresis (ECP), aerosolized cyclosporine, cytolytic anti-lymphocyte therapies, thymoglobulin, total lymphoid irradiation (TLI), pirfenidone, everolimus, sirolimus, rapamycin, inhaled rapamycin, macitentan, prednisone, baricitinib, anti-CD94 monoclonal antibody, aztreonam lysine inhalation, tocilizumab, mesenchymal stem cells, regadenoson, belumosudil, immunoglobulin and/or belatacept.

In some aspects of the methods of treating or preventing transplant rejection in a subject having or at risk of developing transplant rejection, said step of administering a CLAD therapeutic to a subject identified in need thereof comprises administering a higher dose of said CLAD therapeutic than had been administered prior to treating a subject having transplant rejection.

In some aspects of the methods of treating or preventing transplant rejection in a subject having or at risk of developing transplant rejection, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of treating or preventing transplant rejection in a subject having or at risk of developing transplant rejection, the AI2 gene score is used as input for a random forest machine learning model. In some aspects of the methods of treating or preventing transplant rejection in a subject having or at risk of developing transplant rejection, gene expression specific to basal, club, secretory, secretory-ciliated, ciliated, ionocyte, mast, lymphocyte, and/or monocyte cells is used to normalize and improve the accuracy of a classifier.

In some aspects of the methods of treating or preventing transplant rejection in a subject having or at risk of developing transplant rejection, the epithelial subtype genes include one or more of SCGB3A1, MS4A8, KRT5, and CALM1. In some aspects of the methods of treating or preventing transplant rejection in a subject having or at risk of developing transplant rejection, the leukocyte genes include one or more of PTPRC, MARCO, and GNLY.

Disclosed are methods of treating or preventing small airway fibrosis in a subject having or at risk of developing small airway fibrosis comprising: administering a CLAD therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

In some aspects of the methods of treating or preventing small airway fibrosis in a subject having or at risk of developing small airway fibrosis, the AI2 metagene score comprises one or more of an RS1 subscore, RS2 subscore, or RS3 subscore.

In some aspects of the methods of treating or preventing small airway fibrosis in a subject having or at risk of developing small airway fibrosis, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of treating or preventing small airway fibrosis in a subject having or at risk of developing small airway fibrosis, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of treating or preventing small airway fibrosis in a subject having or at risk of developing small airway fibrosis, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of treating or preventing small airway fibrosis in a subject having or at risk of developing small airway fibrosis, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of treating or preventing small airway fibrosis in a subject having or at risk of developing small airway fibrosis, the methods further comprise determining an RS1 subscore, RS2 subscore and/or RS3 subscore.

In some aspects of the methods of treating or preventing small airway fibrosis in a subject having or at risk of developing small airway fibrosis, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

In some aspects of the methods of treating or preventing small airway fibrosis in a subject having or at risk of developing small airway fibrosis, the RS1 subscore comprises expression data of an RS1 gene cluster comprising the genes PSMB9, HLA-A, SAT1, TPM4, MDK, HLA-E, and IRF1.

In some aspects of the methods of treating or preventing small airway fibrosis in a subject having or at risk of developing small airway fibrosis, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F.

In some aspects of the methods of treating or preventing small airway fibrosis in a subject having or at risk of developing small airway fibrosis, the RS2 subscore comprises expression data of an RS2 gene cluster comprising the genes TAP1, CXCL11, GBP4, HLA-F, IDO1, and NLRC5.

In some aspects of the methods of treating or preventing small airway fibrosis in a subject having or at risk of developing small airway fibrosis, the RS3 subscore comprises expression data of an RS3 gene cluster comprising the genes SERPINA3, ADAMDEC1, CXCL13, INPP5D, KLRD1, FCAR, NKG7, and ADORA2A.

In some aspects of the methods of treating or preventing small airway fibrosis in a subject having or at risk of developing small airway fibrosis, the RS3 subscore comprises expression data of an RS3 gene cluster comprising the genes NKG7, KLRD1, ADAMDEC1, and CXCL13.

In some aspects of the methods of treating or preventing small airway fibrosis in a subject having or at risk of developing small airway fibrosis, the RS1 subscore is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of treating or preventing small airway fibrosis in a subject having or at risk of developing small airway fibrosis, the RS2 subscore is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of treating or preventing small airway fibrosis in a subject having or at risk of developing small airway fibrosis, the RS3 subscore is determined by a) normalizing the number of copies of each gene in the RS3 gene cluster by variance stabilizing transformation to obtain a subject's normalized gene count for each gene in the RS3 gene cluster; b) calculating a subject's total normalized gene count by adding the subject's normalized gene counts for each gene in the RS3 gene cluster; c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS3 subscore.

In some aspects of the methods of treating or preventing small airway fibrosis in a subject having or at risk of developing small airway fibrosis, the AI2 metagene score is greater than 0.43. In some aspects of the methods of treating or preventing small airway fibrosis in a subject having or at risk of developing small airway fibrosis, the AI2 metagene score is greater than 0. In some aspects of the methods of treating or preventing small airway fibrosis in a subject having or at risk of developing small airway fibrosis, the AI2 metagene score is between 0 and 1, 0 and 2, 0 and 3, 0 and 4 or 0 and 5. In some aspects of the methods of treating or preventing small airway fibrosis in a subject having or at risk of developing small airway fibrosis, the AI2 metagene score is between 0.43 and 3.

In some aspects of the methods of treating or preventing small airway fibrosis in a subject having or at risk of developing small airway fibrosis, the sample was obtained from small airways. In some aspects of the methods of treating or preventing small airway fibrosis in a subject having or at risk of developing small airway fibrosis, the sample was obtained by a cytology brush. In some aspects of the methods of treating or preventing small airway fibrosis in a subject having or at risk of developing small airway fibrosis, the sample was obtained by bronchoscopy.

In some aspects of the methods of treating or preventing small airway fibrosis in a subject having or at risk of developing small airway fibrosis, the subject's AI2 metagene score was determined by isolating RNA from the sample.

In some aspects of the methods of treating or preventing small airway fibrosis in a subject having or at risk of developing small airway fibrosis, the CLAD therapeutic is macrolide antibiotic azithromycin, cyclosporine, tacrolimus, fundoplication for gastroesophageal reflux, montelukast, extracorporeal photopheresis (ECP), aerosolized cyclosporine, cytolytic anti-lymphocyte therapies, thymoglobulin, total lymphoid irradiation (TLI), pirfenidone, everolimus, sirolimus, rapamycin, inhaled rapamycin, macitentan, prednisone, baricitinib, anti-CD94 monoclonal antibody, aztreonam lysine inhalation, tocilizumab, mesenchymal stem cells, regadenoson, belumosudil, immunoglobulin and/or belatacept.

In some aspects of the methods of treating or preventing small airway fibrosis in a subject having or at risk of developing small airway fibrosis, said step of administering a CLAD therapeutic to a subject identified in need thereof comprises administering a higher dose of said CLAD therapeutic than had been administered prior to treating a subject having small airway fibrosis.

In some aspects of the methods of treating or preventing small airway fibrosis in a subject having or at risk of developing small airway fibrosis, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of treating or preventing small airway fibrosis in a subject having or at risk of developing small airway fibrosis, the AI2 gene score is used as input for a random forest machine learning model. In some aspects of the methods of treating or preventing small airway fibrosis in a subject having or at risk of developing small airway fibrosis, gene expression specific to basal, club, secretory, secretory-ciliated, ciliated, ionocyte, mast, lymphocyte, and/or monocyte cells is used to normalize and improve the accuracy of a classifier.

In some aspects of the methods of treating or preventing small airway fibrosis in a subject having or at risk of developing small airway fibrosis, the epithelial subtype genes include one or more of SCGB3A1, MS4A8, KRT5, and CALM1. In some aspects of the methods of treating or preventing small airway fibrosis in a subject having or at risk of developing small airway fibrosis, the leukocyte genes include one or more of PTPRC, MARCO, and GNLY.

Disclosed are methods of treating or preventing antibody mediated rejection (AMR) in a subject having or at risk of developing AMR comprising: administering an AMR therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

In some aspects of the methods of treating or preventing AMR in a subject having or at risk of developing AMR, the AI2 metagene score comprises one or more of an RS1 subscore, RS2 subscore, or RS3 subscore.

In some aspects of the methods of treating or preventing AMR in a subject having or at risk of developing AMR, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of treating or preventing AMR in a subject having or at risk of developing AMR, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of treating or preventing AMR in a subject having or at risk of developing AMR, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of treating or preventing AMR in a subject having or at risk of developing AMR, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of treating or preventing AMR in a subject having or at risk of developing AMR, the methods further comprise determining an RS1 subscore, RS2 subscore and/or RS3 subscore.

In some aspects of the methods of treating or preventing AMR in a subject having or at risk of developing AMR, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

In some aspects of the methods of treating or preventing AMR in a subject having or at risk of developing AMR, the RS1 subscore comprises expression data of an RS1 gene cluster comprising the genes PSMB9, HLA-A, SAT1, TPM4, MDK, HLA-E, and IRF1.

In some aspects of the methods of treating or preventing AMR in a subject having or at risk of developing AMR, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F.

In some aspects of the methods of treating or preventing AMR in a subject having or at risk of developing AMR, the RS2 subscore comprises expression data of an RS2 gene cluster comprising the genes TAP1, CXCL11, GBP4, HLA-F, IDO1, and NLRC5.

In some aspects of the methods of treating or preventing AMR in a subject having or at risk of developing AMR, the RS3 subscore comprises expression data of an RS3 gene cluster comprising the genes SERPINA3, ADAMDEC1, CXCL13, INPP5D, KLRD1, FCAR, NKG7, and ADORA2A.

In some aspects of the methods of treating or preventing AMR in a subject having or at risk of developing AMR, the RS3 subscore comprises expression data of an RS3 gene cluster comprising the genes NKG7, KLRD1, ADAMDEC1, and CXCL13.

In some aspects of the methods of treating or preventing AMR in a subject having or at risk of developing AMR, the RS1 subscore is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of treating or preventing AMR in a subject having or at risk of developing AMR, the RS2 subscore is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of treating or preventing AMR in a subject having or at risk of developing AMR, the RS3 subscore is determined by a) normalizing the number of copies of each gene in the RS3 gene cluster by variance stabilizing transformation to obtain a subject's normalized gene count for each gene in the RS3 gene cluster; b) calculating a subject's total normalized gene count by adding the subject's normalized gene counts for each gene in the RS3 gene cluster; c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS3 subscore.

In some aspects of the methods of treating or preventing AMR in a subject having or at risk of developing AMR, the AI2 metagene score is greater than 0.43. In some aspects of the methods of treating or preventing AMR in a subject having or at risk of developing AMR, the AI2 metagene score is greater than 0. In some aspects of the methods of treating or preventing AMR in a subject having or at risk of developing AMR, the AI2 metagene score is between 0 and 1, 0 and 2, 0 and 3, 0 and 4 or 0 and 5. In some aspects of the methods of treating or preventing AMR in a subject having or at risk of developing AMR, the AI2 metagene score is between 0.43 and 3.

In some aspects of the methods of treating or preventing AMR in a subject having or at risk of developing AMR, the sample was obtained from small airways. In some aspects of the methods of treating or preventing AMR in a subject having or at risk of developing AMR, the sample was obtained by a cytology brush. In some aspects of the methods of treating or preventing AMR in a subject having or at risk of developing AMR, the sample was obtained by bronchoscopy.

In some aspects of the methods of treating or preventing AMR in a subject having or at risk of developing AMR, the subject's AI2 metagene score was determined by isolating RNA from the sample.

In some aspects of the methods of treating or preventing AMR in a subject having or at risk of developing AMR, the AMR therapeutic is rituximab, intravenous immune globulin (IVIG), plasmapheresis, anti-thymocyte globulin, daratumumab, bortezomib, carfilzomib, tocilizumab, belimumab, C1 esterase inhibitor, plerixafor, a combination of these, or pharmaceuticals of the same classes.

In some aspects of the methods of treating or preventing AMR in a subject having or at risk of developing AMR, said step of administering an AMR therapeutic to a subject identified in need thereof comprises administering a higher dose of said AMR therapeutic than had been administered prior to treating a subject having AMR.

In some aspects of the methods of treating or preventing AMR in a subject having or at risk of developing AMR, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of treating or preventing AMR in a subject having or at risk of developing AMR, the AI2 gene score is used as input for a random forest machine learning model. In some aspects of the methods of treating or preventing AMR in a subject having or at risk of developing AMR, gene expression specific to basal, club, secretory, secretory-ciliated, ciliated, ionocyte, mast, lymphocyte, and/or monocyte cells is used to normalize and improve the accuracy of a classifier.

In some aspects of the methods of treating or preventing AMR in a subject having or at risk of developing AMR, the epithelial subtype genes include one or more of SCGB3A1, MS4A8, KRT5, and CALM1. In some aspects of the methods of treating or preventing AMR in a subject having or at risk of developing AMR, the leukocyte genes include one or more of PTPRC, MARCO, and GNLY.

Disclosed are methods of reducing mortality associated with graft failure in a subject at risk of mortality associated with graft failure comprising: administering a CLAD therapeutic to the subject identified in need thereof, wherein the subject was identified as being in need thereof by determining that the subject has an Airway Inflammation 2 (AI2) metagene score from a sample obtained from the subject which is higher than an AI2 metagene score obtained from a reference population.

In some aspects of the methods of reducing mortality associated with graft failure in a subject at risk of mortality associated with graft failure, the AI2 metagene score comprises one or more of an RS1 subscore, RS2 subscore, or RS3 subscore.

In some aspects of the methods of reducing mortality associated with graft failure in a subject at risk of mortality associated with graft failure, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of reducing mortality associated with graft failure in a subject at risk of mortality associated with graft failure, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of reducing mortality associated with graft failure in a subject at risk of mortality associated with graft failure, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of reducing mortality associated with graft failure in a subject at risk of mortality associated with graft failure, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of reducing mortality associated with graft failure in a subject at risk of mortality associated with graft failure, the methods further comprise determining an RS1 subscore, RS2 subscore and/or RS3 subscore.

In some aspects of the methods of reducing mortality associated with graft failure in a subject at risk of mortality associated with graft failure, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

In some aspects of the methods of reducing mortality associated with graft failure in a subject at risk of mortality associated with graft failure, the RS1 subscore comprises expression data of an RS1 gene cluster comprising the genes PSMB9, HLA-A, SAT1, TPM4, MDK, HLA-E, and IRF1.

In some aspects of the methods of reducing mortality associated with graft failure in a subject at risk of mortality associated with graft failure, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F.

In some aspects of the methods of reducing mortality associated with graft failure in a subject at risk of mortality associated with graft failure, the RS2 subscore comprises expression data of an RS2 gene cluster comprising the genes TAP1, CXCL11, GBP4, HLA-F, IDO1, and NLRC5.

In some aspects of the methods of reducing mortality associated with graft failure in a subject at risk of mortality associated with graft failure, the RS3 subscore comprises expression data of an RS3 gene cluster comprising the genes SERPINA3, ADAMDEC1, CXCL13, INPP5D, KLRD1, FCAR, NKG7, and ADORA2A.

In some aspects of the methods of reducing mortality associated with graft failure in a subject at risk of mortality associated with graft failure, the RS3 subscore comprises expression data of an RS3 gene cluster comprising the genes NKG7, KLRD1, ADAMDEC1, and CXCL13.

In some aspects of the methods of reducing mortality associated with graft failure in a subject at risk of mortality associated with graft failure, the RS1 subscore is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of reducing mortality associated with graft failure in a subject at risk of mortality associated with graft failure, the RS2 subscore is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of reducing mortality associated with graft failure in a subject at risk of mortality associated with graft failure, the RS3 subscore is determined by a) normalizing the number of copies of each gene in the RS3 gene cluster by variance stabilizing transformation to obtain a subject's normalized gene count for each gene in the RS3 gene cluster; b) calculating a subject's total normalized gene count by adding the subject's normalized gene counts for each gene in the RS3 gene cluster; c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS3 subscore.

In some aspects of the methods of reducing mortality associated with graft failure in a subject at risk of mortality associated with graft failure, the AI2 metagene score is greater than 0.43. In some aspects of the methods of reducing mortality associated with graft failure in a subject at risk of mortality associated with graft failure, the AI2 metagene score is greater than 0. In some aspects of the methods of reducing mortality associated with graft failure in a subject at risk of mortality associated with graft failure, the AI2 metagene score is between 0 and 1, 0 and 2, 0 and 3, 0 and 4 or 0 and 5. In some aspects of the methods of reducing mortality associated with graft failure in a subject at risk of mortality associated with graft failure, the AI2 metagene score is between 0.43 and 3.

In some aspects of the methods of reducing mortality associated with graft failure in a subject at risk of mortality associated with graft failure, the sample was obtained from small airways. In some aspects of the methods of reducing mortality associated with graft failure in a subject at risk of mortality associated with graft failure, the sample was obtained by a cytology brush. In some aspects of the methods of reducing mortality associated with graft failure in a subject at risk of mortality associated with graft failure, the sample was obtained by bronchoscopy.

In some aspects of the methods of reducing mortality associated with graft failure in a subject at risk of mortality associated with graft failure, the subject's AI2 metagene score was determined by isolating RNA from the sample.

In some aspects of the methods of reducing mortality associated with graft failure in a subject at risk of mortality associated with graft failure, the CLAD therapeutic is macrolide antibiotic azithromycin, cyclosporine, tacrolimus, fundoplication for gastroesophageal reflux, montelukast, extracorporeal photopheresis (ECP), aerosolized cyclosporine, cytolytic anti-lymphocyte therapies, thymoglobulin, total lymphoid irradiation (TLI), pirfenidone, everolimus, sirolimus, rapamycin, inhaled rapamycin, macitentan, prednisone, baricitinib, anti-CD94 monoclonal antibody, aztreonam lysine inhalation, tocilizumab, mesenchymal stem cells, regadenoson, belumosudil, immunoglobulin and/or belatacept.

In some aspects of the methods of reducing mortality associated with graft failure in a subject at risk of mortality associated with graft failure, said step of administering a CLAD therapeutic to a subject identified in need thereof comprises administering a higher dose of said CLAD therapeutic than had been administered prior to treating a subject having graft failure.

In some aspects of the methods of reducing mortality associated with graft failure in a subject at risk of mortality associated with graft failure, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of reducing mortality associated with graft failure in a subject at risk of mortality associated with graft failure, the AI2 gene score is used as input for a random forest machine learning model. In some aspects of the methods of reducing mortality associated with graft failure in a subject at risk of mortality associated with graft failure, gene expression specific to basal, club, secretory, secretory-ciliated, ciliated, ionocyte, mast, lymphocyte, and/or monocyte cells is used to normalize and improve the accuracy of a classifier.

In some aspects of the methods of reducing mortality associated with graft failure in a subject at risk of mortality associated with graft failure, the epithelial subtype genes include one or more of SCGB3A1, MS4A8, KRT5, and CALM1. In some aspects of the methods of reducing mortality associated with graft failure in a subject at risk of mortality associated with graft failure, the leukocyte genes include one or more of PTPRC, MARCO, and GNLY.

### 2. Methods of Diagnosing

Disclosed are methods of detecting or diagnosing CLAD in a subject at risk of developing CLAD comprising a) determining an AI2 metagene score from a sample of cells from the airway of the subject; and b) comparing the AI2 metagene score to an AI2 metagene score obtained from a reference population; wherein the AI2 metagene score is higher than the AI2 metagene score obtained from the reference population detects or diagnoses CLAD in the subject.

In some aspects of the methods of detecting or diagnosing CLAD in a subject at risk of developing CLAD, the AI2 metagene score comprises one or more of an RS1 subscore, RS2 subscore, or RS3 subscore.

In some aspects of the methods of detecting or diagnosing CLAD in a subject at risk of developing CLAD, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of detecting or diagnosing CLAD in a subject at risk of developing CLAD, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of detecting or diagnosing CLAD in a subject at risk of developing CLAD, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of detecting or diagnosing CLAD in a subject at risk of developing CLAD, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of detecting or diagnosing CLAD in a subject at risk of developing CLAD, the methods further comprise determining an RS1 subscore, RS2 subscore and/or RS3 subscore.

In some aspects of the methods of detecting or diagnosing CLAD in a subject at risk of developing CLAD, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

the RS1 subscore comprises expression data of an RS1 gene cluster comprising the genes PSMB9, HLA-A, SAT1, TPM4, MDK, HLA-E, and IRF1.

In some aspects of the methods of detecting or diagnosing CLAD in a subject at risk of developing CLAD, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F.

the RS2 subscore comprises expression data of an RS2 gene cluster comprising the genes TAP1, CXCL11, GBP4, HLA-F, IDO1, and NLRC5.

In some aspects of the methods of detecting or diagnosing CLAD in a subject at risk of developing CLAD, the RS3 subscore comprises expression data of an RS3 gene cluster comprising the genes SERPINA3, ADAMDEC1, CXCL13, INPP5D, KLRD1, FCAR, NKG7, and ADORA2A.

the RS3 subscore comprises expression data of an RS3 gene cluster comprising the genes NKG7, KLRD1, ADAMDEC1, and CXCL13.

In some aspects of the methods of detecting or diagnosing CLAD in a subject at risk of developing CLAD, the RS1 score is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of detecting or diagnosing CLAD in a subject at risk of developing CLAD, the RS2 score is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of detecting or diagnosing CLAD in a subject at risk of developing CLAD, the RS3 score is determined by a) normalizing the number of copies of each gene in the RS3 gene cluster by variance stabilizing transformation to obtain a subject's normalized gene count for each gene in the RS3 gene cluster; b) calculating a subject's total normalized gene count by adding the subject's normalized gene counts for each gene in the RS3 gene cluster; c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS3 subscore.

In some aspects of the methods of detecting or diagnosing CLAD in a subject at risk of developing CLAD, the AI2 metagene score is greater than 0.43. In some aspects of the methods of detecting or diagnosing CLAD in a subject at risk of developing CLAD, the AI2 metagene score is greater than 0. In some aspects of the methods of detecting or diagnosing CLAD in a subject at risk of developing CLAD, the AI2 metagene score is between 0 and 1, 0 and 2, 0 and 3, 0 and 4 or 0 and 5. In some aspects of the methods of detecting or diagnosing CLAD in a subject at risk of developing CLAD, the AI2 metagene score is between 0.43 and 3.

In some aspects of the methods of detecting or diagnosing CLAD in a subject at risk of developing CLAD, the sample was obtained from small airways. In some aspects of the methods of detecting or diagnosing CLAD in a subject at risk of developing CLAD, the sample was obtained by a cytology brush. In some aspects of the methods of detecting or diagnosing CLAD in a subject at risk of developing CLAD, the sample was obtained by bronchoscopy.

In some aspects of the methods of detecting or diagnosing CLAD in a subject at risk of developing CLAD, the subject's AI2 metagene score was determined by isolating RNA from the sample.

In some aspects of the methods of detecting or diagnosing CLAD in a subject at risk of developing CLAD, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of detecting or diagnosing CLAD in a subject at risk of developing CLAD, the AI2 gene score is used as input for a random forest machine learning model. In some aspects of the methods of detecting or diagnosing CLAD in a subject at risk of developing CLAD, gene expression specific to basal, club, secretory, secretory-ciliated, ciliated, ionocyte, mast, lymphocyte, and/or monocyte cells is used to normalize and improve the accuracy of a classifier.

In some aspects of the methods of detecting or diagnosing CLAD in a subject at risk of developing CLAD, the epithelial subtype genes include one or more of SCGB3A1, MS4A8, KRT5, and CALM1. In some aspects of the methods of detecting or diagnosing CLAD in a subject at risk of developing CLAD, the leukocyte genes include one or more of PTPRC, MARCO, and GNLY.

Disclosed are methods of predicting transplant rejection in a subject at risk of developing transplant rejection comprising: a) determining an AI2 metagene score from a sample of cells from the airway of the subject; and b) comparing the AI2 metagene score to an AI2 metagene score obtained from a reference population; wherein the AI2 metagene score is higher than the AI2 metagene score obtained from the reference population indicates the subject is at a higher risk for developing transplant rejection.

In some aspects of the methods of predicting transplant rejection in a subject at risk of developing transplant rejection, the AI2 metagene score comprises one or more of an RS1 subscore, RS2 subscore, or RS3 subscore.

In some aspects of the methods of predicting transplant rejection in a subject at risk of developing transplant rejection, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of predicting transplant rejection in a subject at risk of developing transplant rejection, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of predicting transplant rejection in a subject at risk of developing transplant rejection, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of predicting transplant rejection in a subject at risk of developing transplant rejection, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of predicting transplant rejection in a subject at risk of developing transplant rejection, the methods further comprise determining an RS1 subscore, RS2 subscore and/or RS3 subscore.

In some aspects of the methods of predicting transplant rejection in a subject at risk of developing transplant rejection, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

the RS1 subscore comprises expression data of an RS1 gene cluster comprising the genes PSMB9, HLA-A, SAT1, TPM4, MDK, HLA-E, and IRF1.

In some aspects of the methods of predicting transplant rejection in a subject at risk of developing transplant rejection, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F.

the RS2 subscore comprises expression data of an RS2 gene cluster comprising the genes TAP1, CXCL11, GBP4, HLA-F, IDO1, and NLRC5.

In some aspects of the methods of predicting transplant rejection in a subject at risk of developing transplant rejection, the RS3 subscore comprises expression data of an RS3 gene cluster comprising the genes SERPINA3, ADAMDEC1, CXCL13, INPP5D, KLRD1, FCAR, NKG7, and ADORA2A.

the RS3 subscore comprises expression data of an RS3 gene cluster comprising the genes NKG7, KLRD1, ADAMDEC1, and CXCL13.

In some aspects of the methods of predicting transplant rejection in a subject at risk of developing transplant rejection, the RS1 subscore is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of predicting transplant rejection in a subject at risk of developing transplant rejection, the RS2 subscore is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of predicting transplant rejection in a subject at risk of developing transplant rejection, the RS3 subscore is determined by a) normalizing the number of copies of each gene in the RS3 gene cluster by variance stabilizing transformation to obtain a subject's normalized gene count for each gene in the RS3 gene cluster; b) calculating a subject's total normalized gene count by adding the subject's normalized gene counts for each gene in the RS3 gene cluster; c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS3 subscore.

In some aspects of the methods of predicting transplant rejection in a subject at risk of developing transplant rejection, the AI2 metagene score is greater than 0.43. In some aspects of the methods of predicting transplant rejection in a subject at risk of developing transplant rejection, the AI2 metagene score is greater than 0. In some aspects of the methods of predicting transplant rejection in a subject at risk of developing transplant rejection, the AI2 metagene score is between 0 and 1, 0 and 2, 0 and 3, 0 and 4 or 0 and 5. In some aspects of the methods of predicting transplant rejection in a subject at risk of developing transplant rejection, the AI2 metagene score is between 0.43 and 3.

In some aspects of the methods of predicting transplant rejection in a subject at risk of developing transplant rejection.

In some aspects of the methods of predicting transplant rejection in a subject at risk of developing transplant rejection, the sample was obtained from small airways. In some aspects of the methods of predicting transplant rejection in a subject at risk of developing transplant rejection, the sample was obtained by a cytology brush. In some aspects of the methods of predicting transplant rejection in a subject at risk of developing transplant rejection, the sample was obtained by bronchoscopy.

In some aspects of the methods of predicting transplant rejection in a subject at risk of developing transplant rejection, the subject's AI2 metagene score was determined by isolating RNA from the sample.

In some aspects of the methods of predicting transplant rejection in a subject at risk of developing transplant rejection, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of predicting transplant rejection in a subject at risk of developing transplant rejection, the AI2 gene score is used as input for a random forest machine learning model. In some aspects of the methods of predicting transplant rejection in a subject at risk of developing transplant rejection, gene expression specific to basal, club, secretory, secretory-ciliated, ciliated, ionocyte, mast, lymphocyte, and/or monocyte cells is used to normalize and improve the accuracy of a classifier.

In some aspects of the methods of transplant rejection in a subject at risk of developing transplant rejection, the epithelial subtype genes include one or more of SCGB3A1, MS4A8, KRT5, and CALM1. In some aspects of the transplant rejection in a subject at risk of developing transplant rejection, the leukocyte genes include one or more of PTPRC, MARCO, and GNLY.

### 3. Methods of Selecting a Therapeutic

Disclosed are methods of identifying an effective therapeutic for treatment of CLAD in a subject comprising determining an RS1 score from a sample from a subject, wherein when the RS1 score from the sample obtained from the subject is higher than an RS1 score obtained from a reference population, thereby identifying an effective therapeutic for treatment of CLAD in the subject, wherein the effective therapeutic is calcineurin inhibitors, mTOR inhibitors, baricitinib and/or belatacept.

In some aspects of the methods of identifying an effective therapeutic for treatment of CLAD in a subject, the methods further comprise administering cyclosporine, tacrolimus, baricitinib and/or belatacept to the subject.

In some aspects of the methods of identifying an effective therapeutic for treatment of CLAD in a subject, the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8.

In some aspects of the methods of identifying an effective therapeutic for treatment of CLAD in a subject, the RS1 subscore is determined by a) normalizing the number of copies of each gene in the RS1 gene cluster by variance stabilizing transformation to obtain a subject's normalized gene count for each gene in the RS1 gene cluster; b) calculating a subject's total normalized gene count by adding the subject's normalized gene counts for each gene in the RS1 gene cluster; c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS1 subscore.

In some aspects of the methods of identifying an effective therapeutic for treatment of CLAD in a subject, the RS1 subscore is greater than 0.43.

In some aspects of the methods of identifying an effective therapeutic for treatment of CLAD in a subject, the sample was obtained from small airways. In some aspects of the methods of identifying an effective therapeutic for treatment of CLAD in a subject, the sample was obtained by a cytology brush. In some aspects of the methods of identifying an effective therapeutic for treatment of CLAD in a subject, the sample was obtained by bronchoscopy.

In some aspects of the methods of identifying an effective therapeutic for treatment of CLAD in a subject, the subject's RS1 subscore was determined by isolating RNA from the sample.

In some aspects of the methods of identifying an effective therapeutic for treatment of CLAD in a subject, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of identifying an effective therapeutic for treatment of CLAD in a subject, the AI2 gene score is used as input for a random forest machine learning model. In some aspects of the methods of identifying an effective therapeutic for treatment of CLAD in a subject, gene expression specific to basal, club, secretory, secretory-ciliated, ciliated, ionocyte, mast, lymphocyte, and/or monocyte cells is used to normalize and improve the accuracy of a classifier.

In some aspects of the methods of identifying an effective therapeutic for treatment of CLAD in a subject, the epithelial subtype genes include one or more of SCGB3A1, MS4A8, KRT5, and CALM1. In some aspects of the methods of identifying an effective therapeutic for treatment of CLAD in a subject, the leukocyte genes include one or more of PTPRC, MARCO, and GNLY.

Disclosed are methods of identifying an effective therapeutic for treatment of CLAD in a subject comprising determining an RS2 score from a sample from a subject, wherein when the RS2 score from the sample obtained from the subject is higher than an RS2 score obtained from a reference population, thereby identifying an effective therapeutic for the treatment of CLAD in the subject, wherein the effective therapeutic is azithromycin, tocilizumab and/or calcineurin inhibitors.

In some aspects of the methods of identifying an effective therapeutic for treatment of CLAD in a subject, the methods further comprise administering azithromycin, tocilizumab and/or calcineurin inhibitors to the subject.

In some aspects of the methods of identifying an effective therapeutic for treatment of CLAD in a subject, the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F. In some aspects of the methods of identifying an effective therapeutic for treatment of CLAD in a subject, the RS2 subscore is determined by a) normalizing the number of copies of each gene in the RS2 gene cluster by variance stabilizing transformation to obtain a subject's normalized gene count for each gene in the RS2 gene cluster; b) calculating a subject's total normalized gene count by adding the subject's normalized gene counts for each gene in the RS2 gene cluster; c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the RS2 subscore.

In some aspects of the methods of identifying an effective therapeutic for treatment of CLAD in a subject, the RS2 subscore is greater than 0.43.

In some aspects of the methods of identifying an effective therapeutic for treatment of CLAD in a subject, the sample was obtained from small airways. In some aspects of the methods of identifying an effective therapeutic for treatment of CLAD in a subject, the sample was obtained by a cytology brush. In some aspects of the methods of identifying an effective therapeutic for treatment of CLAD in a subject, the sample was obtained by bronchoscopy.

In some aspects of the methods of identifying an effective therapeutic for treatment of CLAD in a subject, the subject's RS2 subscore was determined by isolating RNA from the sample.

In some aspects of the methods of identifying an effective therapeutic for treatment of CLAD in a subject, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of identifying an effective therapeutic for treatment of CLAD in a subject, the AI2 gene score is used as input for a random forest machine learning model. In some aspects of the methods of identifying an effective therapeutic for treatment of CLAD in a subject, gene expression specific to basal, club, secretory, secretory-ciliated, ciliated, ionocyte, mast, lymphocyte, and/or monocyte cells is used to normalize and improve the accuracy of a classifier.

In some aspects of the methods of identifying an effective therapeutic for treatment of CLAD in a subject, the epithelial subtype genes include one or more of SCGB3A1, MS4A8, KRT5, and CALM1. In some aspects of the methods of identifying an effective therapeutic for treatment of CLAD in a subject, the leukocyte genes include one or more of PTPRC, MARCO, and GNLY.

Disclosed are methods of identifying an effective therapeutic for treatment of CLAD in a subject comprising determining an RS3 score from a sample from a subject, wherein if the RS3 score from the sample obtained from the subject is higher than an RS3 score obtained from a reference population, thereby identifying an effective therapeutic for treatment of CLAD in the subject, wherein the effective therapeutic is extracorporeal photopheresis (ECP), lymphoid irradiation (TLI), anti-human thymocyte immunoglobulin (Thymoglobulin®), anti-CD94 monoclonal antibody and/or belumosudil.

In some aspects of the methods of identifying an effective therapeutic for treatment of CLAD in a subject, the methods further comprise administering ECP, TLI, Thymoglobulin®, anti-CD94 monoclonal antibody and/or belumosudil to the subject.

In some aspects of the methods of identifying an effective therapeutic for treatment of CLAD in a subject, the RS3 subscore comprises expression data of an RS3 gene cluster comprising the genes SERPINA3, ADAMDEC1, CXCL13, INPP5D, KLRD1, FCAR, NKG7, and ADORA2A.

In some aspects of the methods of identifying an effective therapeutic for treatment of CLAD in a subject, the RS3 subscore is determined by

In some aspects of the methods of identifying an effective therapeutic for treatment of CLAD in a subject, the RS3 subscore is greater than 0.43.

In some aspects of the methods of identifying an effective therapeutic for treatment of CLAD in a subject, the sample was obtained from small airways. In some aspects of the methods of identifying an effective therapeutic for treatment of CLAD in a subject, the sample was obtained by a cytology brush. In some aspects of the methods of identifying an effective therapeutic for treatment of CLAD in a subject, the sample was obtained by bronchoscopy.

In some aspects of the methods of identifying an effective therapeutic for treatment of CLAD in a subject, the subject's RS3 subscore was determined by isolating RNA from the sample.

In some aspects of the methods of identifying an effective therapeutic for treatment of CLAD in a subject, the AI2, RS1, RS2 and/or RS3 score is determined from basal, club, secretory, secretory-ciliated, ciliated, ionocyte, mast, lymphocyte, and/or monocyte cells in the small airways.

### 4. Methods of Screening

Disclosed are methods of screening for a candidate therapeutic that can treat CLAD in a subject having or at risk of developing CLAD comprising a) determining a first AI2 metagene score from a sample of cells from the subject having or at risk of developing CLAD; b) adding a candidate therapeutic to the sample of cells; c) determining a second AI2 metagene score from the sample after incubating the sample of cells with the candidate therapeutic; and d) determining the candidate therapeutic treats CLAD when the second AI2 metagene score is lower than the first AI2 metagene score.

In some aspects of the methods of screening a candidate therapeutic that can treat CLAD in a subject having or at risk of developing CLAD, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of screening a candidate therapeutic that can treat CLAD in a subject having or at risk of developing CLAD, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of screening a candidate therapeutic that can treat CLAD in a subject having or at risk of developing CLAD, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of screening a candidate therapeutic that can treat CLAD in a subject having or at risk of developing CLAD, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of screening a candidate therapeutic that can treat CLAD in a subject having or at risk of developing CLAD, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of screening a candidate therapeutic that can treat CLAD in a subject having or at risk of developing CLAD, the AI2 gene score is used as input for a random forest machine learning model. In some aspects of the methods of screening a candidate therapeutic that can treat CLAD in a subject having or at risk of developing CLAD, gene expression specific to basal, club, secretory, secretory-ciliated, ciliated, ionocyte, mast, lymphocyte, and/or monocyte cells is used to normalize and improve the accuracy of a classifier.

In some aspects of the methods of screening a candidate therapeutic that can treat CLAD in a subject having or at risk of developing CLAD, the epithelial subtype genes include one or more of SCGB3A1, MS4A8, KRT5, and CALM1. In some aspects of the methods of screening a candidate therapeutic that can treat CLAD in a subject having or at risk of developing CLAD, the leukocyte genes include one or more of PTPRC, MARCO, and GNLY.

Disclosed are methods of screening whether a subject having CLAD is responsive to a therapeutic comprising a) determining a first AI2 metagene score from a first sample of cells from the subject having CLAD; b) administering the therapeutic to the subject; c) determining a second AI2 metagene score from a second sample of cells taken after administering the therapeutic to the subject; d) determining the subject is responsive to the therapeutic when the second AI2 metagene score is lower than the first AI2 metagene score.

In some aspects of the methods of screening whether a subject having CLAD is responsive to a therapeutic, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of screening whether a subject having CLAD is responsive to a therapeutic, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of screening whether a subject having CLAD is responsive to a therapeutic, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of screening whether a subject having CLAD is responsive to a therapeutic, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of screening whether a subject having CLAD is responsive to a therapeutic, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of screening whether a subject having CLAD is responsive to a therapeutic, the AI2 gene score is used as input for a random forest machine learning model. In some aspects of the methods of screening whether a subject having CLAD is responsive to a therapeutic, gene expression specific to basal, club, secretory, secretory-ciliated, ciliated, ionocyte, mast, lymphocyte, and/or monocyte cells is used to normalize and improve the accuracy of a classifier.

In some aspects of the methods of screening whether a subject having CLAD is responsive to a therapeutic, the epithelial subtype genes include one or more of SCGB3A1, MS4A8, KRT5, and CALM1. In some aspects of the methods of screening whether a subject having CLAD is responsive to a therapeutic, the leukocyte genes include one or more of PTPRC, MARCO, and GNLY.

Disclosed are methods of identifying a subject for a clinical study for a CLAD therapeutic, comprising a) determining an AI2 metagene score from a sample of cells from the subject; wherein when the AI2 metagene score is above 0, the subject is appropriate for the clinical study for a CLAD therapeutic.

In some aspects of the methods of identifying a subject for a clinical study for a CLAD therapeutic, when the AI2 metagene score is above 0.43, the subject is appropriate for the clinical study for a CLAD therapeutic.

In some aspects of the methods of identifying a subject for a clinical study for a CLAD therapeutic, when the AI2 metagene score is below 0, the subject is not appropriate for the clinical study for a CLAD therapeutic.

In some aspects of the methods of identifying a subject for a clinical study for a CLAD therapeutic, when the AI2 metagene score is below −0.43, the subject is not appropriate for the clinical study for a CLAD therapeutic.

In some aspects of the methods of identifying a subject for a clinical study for a CLAD therapeutic, the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A.

In some aspects of the methods of identifying a subject for a clinical study for a CLAD therapeutic, the AI2 metagene score comprises expression data from an AI2 metagene comprising the genes CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, and UBD.

In some aspects of the methods of identifying a subject for a clinical study for a CLAD therapeutic, the AI2 metagene score comprises expression data from an AI2 metagene comprising the genes ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4.

In some aspects of the methods of identifying a subject for a clinical study for a CLAD therapeutic, the AI2 metagene score is determined by a) normalizing the number of copies of each gene of the AI2 metagene by variance stabilizing transformation to obtain a subject's normalized gene count for each gene in the AI2 metagene; b) calculating a subject's total normalized gene count by adding the subject's normalized gene counts for each gene in the AI2 metagene; c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises: i) subtracting the reference population mean from the subject's total normalized gene count to obtain a numerator; and ii) dividing the numerator by the reference population standard deviation; thereby determining the AI2 metagene score.

In some aspects of the methods of identifying a subject for a clinical study for a CLAD therapeutic, the AI2, RS1, RS2 and/or RS3 score is determined from basal, club, secretory, secretory-ciliated, ciliated, ionocyte, mast, lymphocyte, and/or monocyte cells in the small airways.

In some aspects of the methods of identifying a subject for a clinical study for a CLAD therapeutic, the AI2 gene score is used as input for a random forest machine learning model. In some aspects of the methods of identifying a subject for a clinical study for a CLAD therapeutic, gene expression specific to basal, club, secretory, secretory-ciliated, ciliated, ionocyte, mast, lymphocyte, and/or monocyte cells is used to normalize and improve the accuracy of a classifier.

In some aspects of the methods of identifying a subject for a clinical study for a CLAD therapeutic, the epithelial subtype genes include one or more of SCGB3A1, MS4A8, KRT5, and CALM1. In some aspects of the methods of identifying a subject for a clinical study for a CLAD therapeutic, the leukocyte genes include one or more of PTPRC, MARCO, and GNLY.

### 5. Administration

The disclosed methods can include one or more of the types of administration disclosed herein.

In the methods described herein, administration or delivery of the therapeutics to a subject can be via a variety of mechanisms. For example, the therapeutic can be formulated as a pharmaceutical composition.

Pharmaceutical compositions can be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising compositions and instructions for carrying out the claimed methods.

### D. Machine Learning Approaches

The present disclosure relates to methods for treating lung conditions in transplant recipients, methods for diagnosing lung conditions in transplant recipients, and methods of screening lung transplant recipients. It is to be appreciated that any of the methods disclosed herein can utilize the machine learning approach described below.

In some implementations, the methods disclosed herein may utilize a random forest machine learning model with an AI2 gene set as input to classify and diagnose CLAD or ALAD. The AI2 gene set may comprise a collection of genes identified as relevant markers for assessing lung allograft health and function.

The disclosed methods may be applied to diagnose Acute Lung Allograft Dysfunction (ALAD) in lung transplant recipients. ALAD may be characterized by a decline in lung function over a relatively short period, which in some cases may progress to CLAD if left untreated. Early and accurate diagnosis of ALAD may allow for timely intervention and improved patient outcomes.

In some cases, the methods described herein may incorporate cell type-specific gene expression data to enhance diagnostic accuracy. This approach may account for variations in cell populations present in biological samples, improving the robustness and reliability of the diagnostic predictions.

The AI2 gene set may be used as a diagnostic tool for assessing lung allograft dysfunction. This gene set may comprise a collection of genes associated with immune response and inflammation in the context of lung transplantation.

In some cases, the AI2 gene set may be positively correlated with myeloid- and lymphoid-associated genes. Conversely, the AI2 gene set may be negatively correlated with epithelial-associated genes. This correlation pattern may provide insights into the cellular processes involved in lung allograft dysfunction.

The AI2 gene set may be expressed in multiple airway brush cell subsets. These subsets may include club cells, T cells, myeloid cells, and certain subsets of basal and ciliated cells. The expression of the AI2 gene set across these diverse cell types may indicate its broad relevance in assessing lung health post-transplantation.

In some implementations, the AI2 gene set may be used in conjunction with a reference gene set. This reference gene set may include genes that are highly expressed and negatively correlated with the AI2 score. Examples of such reference genes may include SCGB3A1 (associated with secretory/club cells), MS4A8 (associated with ciliated cells), KRT5 (associated with basal cells), and CALM1 (associated with transitional epithelial cells).

The AI2 gene set, when used in combination with appropriate reference genes, may allow for the development of assays based on unnormalized counts. This approach may facilitate the use of more clinically applicable sequencing technologies, such as quantitative PCR, reverse transcription-loop mediated isothermal amplification (LAMP), or digital RNA counting methods.

In some cases, the AI2 gene set may be used to calculate an AI2 score. This score may be highest in recipients with Acute Lung Allograft Dysfunction (ALAD) who subsequently decline in lung function. The AI2 score may show statistical significance in various epithelial cell subsets, including basal, club, ciliated, and transitional epithelial cells.

The AI2 gene set may serve as a valuable tool for predicting outcomes in lung transplant recipients. When used in combination with appropriate reference genes and analytical methods, the AI2 gene set may contribute to improved diagnosis and monitoring of lung allograft dysfunction.

The method may utilize a random forest machine learning model to process and analyze the AI2 gene set data for diagnosing CLAD or ALAD. Random forest is an ensemble learning technique that constructs multiple decision trees and combines their outputs to make predictions.

In some implementations, the random forest model may comprise 500 individual decision trees. Each tree in the forest may be trained on a bootstrap sample of the training data, drawn with replacement. This approach, known as bagging (bootstrap aggregating), helps to reduce overfitting and improve the model's generalization capabilities.

The random forest model may take the AI2 gene set as input features. In some cases, the input may be preprocessed by applying a logarithmic transformation to the gene expression counts, specifically log(counts+1). This transformation may help to normalize the data and reduce the impact of extreme values.

During the training process, each decision tree in the random forest may be constructed by recursively splitting the data based on the input features. At each node, a subset of features may be randomly selected, and the best split among these features may be chosen based on a criterion such as Gini impurity or information gain.

When making predictions, the random forest model may aggregate the outputs of all individual trees. For classification tasks, such as diagnosing CLAD or ALAD, this aggregation may involve a majority vote among the trees. The final prediction may be determined by the class that receives the most votes across all trees in the forest.

The use of multiple trees and random feature selection at each node may contribute to the model's robustness and ability to capture complex relationships in the gene expression data. This approach may help to mitigate the impact of noise or irrelevant features in the AI2 gene set.

In some cases, the random forest model may provide feature importance scores, indicating the relative contribution of each gene in the AI2 set to the classification decision. This information may be valuable for understanding the biological mechanisms underlying CLAD or ALAD and for refining the gene set in future iterations of the diagnostic method.

The random forest model's performance may be evaluated using metrics such as area under the receiver operating characteristic curve (AUC-ROC), accuracy, precision, and recall. These metrics may help assess the model's ability to correctly classify CLAD or ALAD cases and controls.

The method may utilize cell type-specific genes for normalization to improve the accuracy of the classifier. In some cases, a reference gene set may be employed to account for variations in cell populations present in airway brush samples. This reference gene set may include genes that are highly expressed and negatively correlated with the AI2 score, reflecting several epithelial subtypes.

The reference gene set may comprise SCGB3A1, MS4A8, KRT5, and CALM1. SCGB3A1 may be associated with secretory or club cells, MS4A8 may be associated with ciliated cells, KRT5 may be associated with basal cells, and CALM1 may be associated with transitional epithelial cells. These genes may represent different epithelial subtypes commonly found in airway brush samples.

In some implementations, single cell RNA sequencing data may be used to validate the reference gene set. This approach may allow for a more detailed examination of gene expression patterns across different cell types present in airway brush samples. The single cell RNA sequencing data may be collected from lung transplant recipients, including those with Acute Lung Allograft Dysfunction (ALAD) and stable controls.

The reference gene set may be used to calculate a reference score, which may be negatively associated with Chronic Lung Allograft Dysfunction (CLAD). In some cases, the reference score may be particularly significant in club cells, consistent with literature linking club cell dysfunction to CLAD.

By incorporating these cell type-specific genes, the method may normalize the AI2 gene scores and account for variations in cell populations. This normalization process may improve the accuracy of the classifier in diagnosing CLAD or ALAD. In some implementations, the difference between the AI2 gene set score and the reference gene set score may provide the most accurate prediction of ALAD outcomes.

The use of this reference gene set may allow for the development of assays based on unnormalized counts. This approach may facilitate the translation of RNA sequencing findings to more clinically applicable technologies, such as quantitative PCR, reverse transcription-loop mediated isothermal amplification (LAMP), or digital RNA counting methods.

In some implementations, additional reference genes may be incorporated into the diagnostic method to enhance classification accuracy. These additional reference genes may represent various cell types commonly found in airway brush samples, including secretory, ciliated, basal, and leukocyte cells.

The reference gene set may be expanded to include SCGB3A1 for secretory cells, MS4A8 and CALM1 for ciliated cells, KRT5 and TP63 for basal cells, and PTPRC, MARCO, and GNLY for leukocyte cells. This expanded set of reference genes may provide a more comprehensive representation of the cellular composition in airway brush samples.

SCGB3A1, previously mentioned as a marker for secretory or club cells, may be retained in the expanded reference gene set. MS4A8 and CALM1 may serve as markers for ciliated cells, offering complementary information about this cell type. KRT5, previously included as a basal cell marker, may be supplemented with TP63, which may also be associated with basal epithelial cells.

The addition of leukocyte-specific genes, namely PTPRC, MARCO, and GNLY, may allow for better quantification of immune cell populations in the samples. PTPRC, also known as CD45, may be a general marker for leukocytes. MARCO may be associated with macrophages, while GNLY may be expressed in certain subsets of T cells and natural killer cells.

In some cases, the inclusion of these additional reference genes in the random forest machine learning model may improve the model's ability to distinguish between different cell types present in the airway brush samples. This enhanced cellular resolution may contribute to more accurate classification of Chronic Lung Allograft Dysfunction (CLAD) or Acute Lung Allograft Dysfunction (ALAD) cases.

The expanded reference gene set may help normalize the AI2 gene expression data by accounting for variations in cell type composition across different samples. This normalization process may reduce noise and improve the signal-to-noise ratio in the gene expression data, leading to more robust and reliable diagnostic predictions.

In some implementations, the relative importance of these additional reference genes in the classification process may be assessed. This assessment may provide insights into which cell types or cellular processes are most relevant for distinguishing between CLAD or ALAD cases and controls.

The use of this expanded reference gene set may contribute to the development of more sophisticated and accurate diagnostic tools for lung allograft dysfunction. By accounting for a broader range of cell types, these tools may offer a more nuanced understanding of the cellular changes associated with CLAD or ALAD.

The method may utilize unnormalized counts from clinically applicable sequencing technologies as input for the random forest machine learning model. In some cases, these unnormalized counts may be derived from quantitative PCR, reverse transcription-loop mediated isothermal amplification (LAMP), or digital RNA counting methods. The use of unnormalized counts may facilitate the translation of RNA sequencing findings to more clinically relevant and accessible technologies.

In some implementations, the random forest model may be trained using a modified leave-one-out cross validation strategy. This approach may help ensure that the model is not trained on data from the same subject as the test data, reducing bias and improving generalization. The modified leave-one-out cross validation strategy may involve iteratively training the model on all but one subject's data, then testing on the held-out subject's data.

The training process may involve constructing multiple decision trees, each trained on a bootstrap sample of the training data. In some cases, 500 trees may be used in the random forest model. Each tree may be built by recursively splitting the data based on randomly selected subsets of features at each node.

During the modified leave-one-out cross validation, the model may be trained multiple times, each time excluding data from a different subject. This approach may help assess the model's performance on unseen data and provide a more robust estimate of its generalization capabilities.

The input features for the random forest model may include the log-transformed gene expression counts from the AI2 gene set, as well as the reference genes for various cell types. In some implementations, the log transformation may be applied as log(counts+1) to handle zero counts and reduce the impact of extreme values.

The modified leave-one-out cross validation strategy may address overfitting issues and provide a more accurate assessment of the model's performance in real-world scenarios. This approach may be particularly valuable when working with limited sample sizes, as is often the case in clinical studies involving lung transplant recipients.

The method for diagnosing Chronic Lung Allograft Dysfunction (CLAD) or Acute Lung Allograft Dysfunction (ALAD) may integrate multiple components to achieve accurate classification. These components may include the AI2 gene set, a random forest machine learning model, cell type-specific genes, and additional reference genes.

In some implementations, the diagnostic process may begin with the collection of gene expression data from airway brush samples obtained from lung transplant recipients. This data may include expression levels for genes in the AI2 gene set, as well as cell type-specific genes and additional reference genes.

The AI2 gene set may serve as the primary input for the random forest machine learning model. This gene set may comprise markers associated with immune response and inflammation in the context of lung transplantation. The expression levels of these genes may be quantified using clinically applicable sequencing technologies, such as quantitative PCR, reverse transcription-loop mediated isothermal amplification (LAMP), or digital RNA counting methods.

In some cases, the gene expression data may be preprocessed before being input into the random forest model. This preprocessing may involve applying a logarithmic transformation to the gene expression counts, specifically log(counts+1). This transformation may help normalize the data and reduce the impact of extreme values.

The random forest model may be trained to classify samples as CLAD, ALAD, or control based on the AI2 gene set expression patterns. During the training process, the model may construct multiple decision trees, each trained on a bootstrap sample of the training data. This approach, known as bagging, may help reduce overfitting and improve the model's generalization capabilities.

To enhance the accuracy of the classifier, cell type-specific genes may be incorporated into the analysis. These genes may include SCGB3A1 (associated with secretory cells), MS4A8 and CALM1 (associated with ciliated cells), KRT5 and TP63 (associated with basal cells), and PTPRC, MARCO, and GNLY (associated with leukocyte cells). The inclusion of these genes may help normalize the AI2 gene scores and account for variations in cell populations across different samples.

In some implementations, the random forest model may use both the AI2 gene set and the cell type-specific genes as input features. This combined approach may allow the model to consider both the immune response signals captured by the AI2 gene set and the cellular composition information provided by the cell type-specific genes.

The model may process the input data through its multiple decision trees, with each tree making a classification decision. The final classification may be determined by aggregating the outputs of all trees, typically through a majority vote mechanism.

In some cases, the model may provide feature importance scores, indicating the relative contribution of each gene to the classification decision. This information may be valuable for understanding the biological mechanisms underlying CLAD or ALAD and for refining the gene set in future iterations of the diagnostic method.

The output of the random forest model may be a classification of the sample as CLAD, ALAD, or control. Additionally, the model may provide a probability score associated with each classification, indicating the confidence level of the prediction.

In some implementations, the diagnostic process may involve multiple stages of analysis. For example, an initial classification may be made based on the AI2 gene set alone, followed by a refined classification that incorporates the cell type-specific genes. This multi-stage approach may allow for a more nuanced assessment of the sample's status.

The integration of these various components—the AI2 gene set, random forest model, cell type-specific genes, and additional reference genes—may result in a comprehensive diagnostic tool for CLAD and ALAD. This integrated approach may leverage the strengths of each component to provide a more accurate and robust classification of lung allograft dysfunction.

The performance of the Chronic Lung Allograft Dysfunction (CLAD) classifier may be evaluated using various statistical methods to assess its accuracy and reliability. In some cases, the area under the receiver operating characteristic curve (AUC-ROC) may be calculated to quantify the classifier's performance.

The AUC-ROC may provide a measure of the classifier's ability to distinguish between CLAD cases and controls across different classification thresholds. A higher AUC value may indicate better discriminative power of the classifier, with a value of 1.0 representing perfect classification and 0.5 representing performance no better than random chance.

In some implementations, the DeLong method may be employed to assess the statistical significance of the AUC-ROC and to calculate confidence intervals. The DeLong method may account for the correlation between multiple ROC curves derived from the same set of cases and controls, providing a more accurate estimate of the standard error.

The calculation of AUC-ROC may involve plotting the true positive rate (sensitivity) against the false positive rate (1—specificity) at various threshold settings. The resulting curve may be used to compute the AUC, which may represent the probability that the classifier will rank a randomly chosen positive instance higher than a randomly chosen negative instance.

In some cases, the performance of different classifiers or variations of the CLAD diagnostic method may be compared using their respective AUC-ROC values. For instance, the AUC of the AI2 score alone may be compared to the AUC of the random forest model trained on unnormalized counts, as well as the AUC of the model incorporating cell type reference genes.

The DeLong method may be used to test for statistically significant differences between these AUC values. This approach may help determine whether the addition of cell type reference genes or the use of machine learning techniques significantly improves the classifier's performance compared to simpler methods.

In some implementations, confidence intervals for the AUC-ROC may be calculated using the DeLong method. These confidence intervals may provide a range of plausible values for the true AUC, accounting for the uncertainty in the estimate due to sample size and variability.

The performance evaluation process may also involve assessing the classifier's sensitivity and specificity at various decision thresholds. This analysis may help in selecting an optimal threshold for clinical use, balancing the trade-off between false positives and false negatives.

In some cases, additional performance metrics such as positive predictive value, negative predictive value, and overall accuracy may be calculated to provide a more comprehensive assessment of the classifier's performance in diagnosing CLAD.

The performance evaluation methods described may help validate the effectiveness of the CLAD diagnostic approach and provide quantitative evidence of its accuracy and reliability. These assessments establish the clinical utility of the diagnostic method and may guide its implementation in lung transplant recipient care.

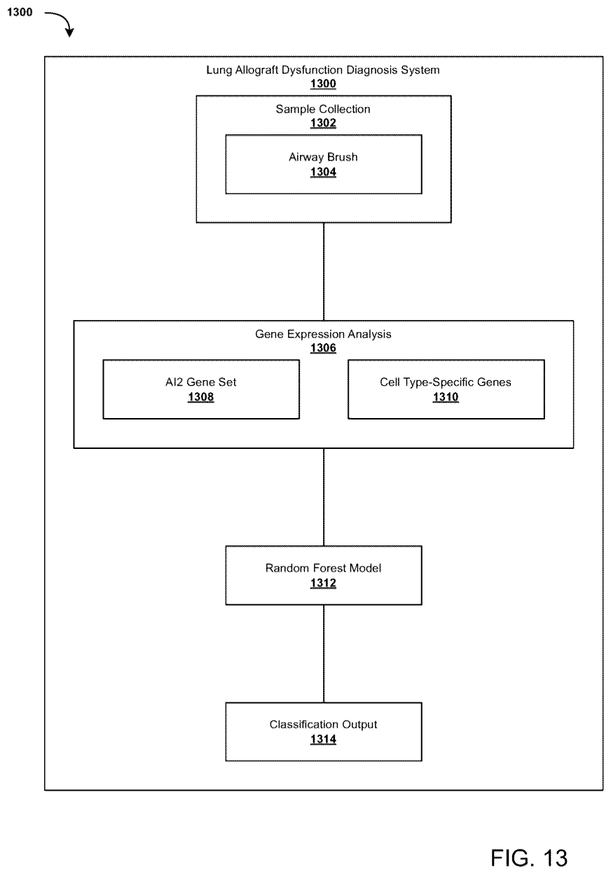

FIG. 13 illustrates a flowchart for a lung allograft dysfunction diagnosis system 1300. The system 1300 includes multiple processing steps arranged in a sequential flow.

The system 1300 begins with a sample collection step 1302, which contains an airway brush step 1304 for obtaining biological samples. From the sample collection, the process flows to a gene expression analysis step 1306.

The gene expression analysis step 1306 comprises two parallel components: an AI2 gene set step 1308 and a cell type-specific genes step 1310. These components process different types of genetic information obtained from the samples.

The AI2 gene set step 1308 may involve processing and analyzing the expression data for a specific set of genes identified as relevant markers for assessing lung allograft health and function. This step may include several sub-processes to prepare the AI2 gene expression data for input into the random forest model.

In some implementations, the AI2 gene set step 1308 may begin with data normalization to account for variations in sample preparation and sequencing depth. This normalization process may involve techniques such as quantile normalization or scaling to a reference gene set.

The AI2 gene set step 1308 may also include a quality control phase where outliers or samples with poor sequencing quality are identified and may be excluded from further analysis. This quality control process may help ensure the reliability of the input data for the machine learning model.

In some cases, the AI2 gene set step 1308 may involve calculating an AI2 score based on the expression levels of the genes in the set. This score may be derived using various methods, such as taking the mean or median expression across the gene set, or applying a weighted sum based on the relative importance of each gene.

The AI2 gene set step 1308 may also include a feature selection or dimensionality reduction process. This may involve identifying the most informative genes within the AI2 set for distinguishing between CLAD, ALAD, and control samples. Techniques such as principal component analysis (PCA) or recursive feature elimination may be employed in this sub-step.

In some implementations, the AI2 gene set step 1308 may incorporate a log transformation of the gene expression counts, such as log(counts+1), to address the skewed distribution often observed in gene expression data and to make the data more suitable for input into the random forest model.

The AI2 gene set step 1308 may also involve scaling or standardizing the gene expression values to ensure that all features contribute equally to the model's decision-making process. This may be particularly important when combining the AI2 gene set data with other input features in subsequent steps of the analysis pipeline.

The cell type-specific genes step 1310 may involve processing and analyzing gene expression data for markers associated with various cell types present in airway brush samples. This step may be crucial for normalizing the AI2 gene set data and accounting for variations in cellular composition across different samples.

In some implementations, the cell type-specific genes step 1310 may begin with the selection of appropriate marker genes for different cell types. This selection may include genes such as SCGB3A1 for secretory cells, MS4A8 and CALM1 for ciliated cells, KRT5 and TP63 for basal cells, and PTPRC, MARCO, and GNLY for leukocyte cells. The choice of marker genes may be based on prior knowledge of cell-specific expression patterns and literature evidence.

The cell type-specific genes step 1310 may involve quantifying the expression levels of these marker genes using the same sequencing technology employed for the AI2 gene set. In some cases, this may include techniques such as quantitative PCR, reverse transcription-loop mediated isothermal amplification (LAMP), or digital RNA counting methods.

Data preprocessing may be performed as part of the cell type-specific genes step 1310. This preprocessing may include normalization techniques to account for technical variations in sample preparation and sequencing. In some implementations, the preprocessing may involve log transformation of gene expression counts, similar to the approach used for the AI2 gene set.

The cell type-specific genes step 1310 may include a quality control phase to identify and potentially exclude outlier samples or genes with unreliable expression measurements. This quality control process may help ensure the robustness of the cell type composition estimates.

In some cases, the cell type-specific genes step 1310 may involve calculating relative abundances or proportions of different cell types based on the expression levels of the marker genes. Various computational methods, such as deconvolution algorithms or marker gene averaging, may be employed to estimate cell type proportions from the gene expression data.

The cell type-specific genes step 1310 may also include a feature engineering phase, where derived features based on cell type proportions or expression ratios between different marker genes are calculated. These derived features may provide additional information about the cellular composition of the samples and may be used as inputs for the random forest model.

In some implementations, the cell type-specific genes step 1310 may involve integrating the cell type composition data with the AI2 gene set data. This integration may include techniques such as batch correction or data harmonization to ensure compatibility between the two data types.

The output of the cell type-specific genes step 1310 may include processed gene expression data for the cell type markers, estimated cell type proportions, and potentially derived features based on cellular composition. This output may be combined with the processed AI2 gene set data to serve as input for the subsequent random forest model step 1312.

Following the gene expression analysis, the system 1300 proceeds to a random forest model step 1312. This step processes the combined genetic information from both the AI2 gene set and cell type-specific genes.

The random forest model step 1312 may involve several key processes and considerations in processing the combined genetic information from the AI2 gene set and cell type-specific genes. In some implementations, this step may begin with data integration, combining the preprocessed inputs from the AI2 gene set step 1308 and the cell type-specific genes step 1310 into a unified feature set.

The random forest model may be constructed using an ensemble of decision trees, typically numbering in the hundreds or thousands. Each tree in the forest may be trained on a bootstrap sample of the input data, a technique known as bagging. This approach may help reduce overfitting and improve the model's generalization capabilities.

In some cases, the random forest model step 1312 may incorporate feature importance analysis. This process may involve calculating the mean decrease in impurity or the mean decrease in accuracy for each input feature across all trees in the forest. These importance scores may provide insights into which genes or cell type markers are most informative for distinguishing between CLAD, ALAD, and control samples.

The model may employ a technique called random feature selection at each node split. Instead of considering all available features at each split, the model may randomly select a subset of features. This approach may help to decorrelate the trees and further improve the model's robustness.

In some implementations, the random forest model step 1312 may include hyperparameter tuning. This process may involve optimizing parameters such as the number of trees, the maximum depth of each tree, the minimum number of samples required to split an internal node, and the minimum number of samples required to be at a leaf node. Techniques such as grid search, random search, or Bayesian optimization may be employed for this purpose.

The random forest model may use a voting mechanism to aggregate predictions from individual trees. For classification tasks, such as distinguishing between CLAD, ALAD, and control samples, the model may use majority voting, where the class predicted by the majority of trees becomes the final prediction.

In some cases, the random forest model step 1312 may incorporate techniques to handle class imbalance, which may occur if one class (e.g., control samples) is significantly more prevalent in the dataset than others. Methods such as class weighting or synthetic minority over-sampling technique (SMOTE) may be employed to address this issue.

The model may also provide probability estimates for each class, which may be derived from the proportion of trees voting for each class. These probability estimates may offer additional information about the model's confidence in its predictions.

In some implementations, the random forest model step 1312 may include a cross-validation procedure to assess the model's performance and generalization capabilities. This may involve techniques such as k-fold cross-validation or leave-one-out cross-validation, depending on the size and structure of the dataset.

The output of the random forest model step 1312 may include class predictions for each sample, probability estimates for each class, feature importance scores, and various performance metrics such as accuracy, precision, recall, and F1 score. This comprehensive output may serve as input for the subsequent classification output step 1314, providing a robust basis for diagnosing lung allograft dysfunction.

The system 1300 concludes with a classification output step 1314, where the processed data from the random forest model produces diagnostic results. The flowchart shows a linear progression from sample collection through analysis to final classification output.

The classification output step 1314 may involve processing and interpreting the results generated by the random forest model to produce a final diagnostic classification for lung allograft dysfunction. In some implementations, this step may begin by aggregating the predictions and probability estimates from all trees in the random forest ensemble.

The system may apply a threshold to the aggregated probability estimates to determine the final classification. For instance, if the probability of CLAD or ALAD exceeds a predetermined threshold, the sample may be classified as positive for that condition. The threshold may be adjustable based on the desired balance between sensitivity and specificity.

In some cases, the classification output step 1314 may incorporate a multi-class classification scheme, distinguishing between CLAD, ALAD, and control samples. The system may use techniques such as one-vs-rest or one-vs-one classification strategies to handle this multi-class scenario.

The classification output step 1314 may also include a confidence score associated with each prediction. This score may be derived from the probability estimates provided by the random forest model and may indicate the reliability of the classification result.

In some implementations, the classification output step 1314 may involve post-processing of the model's predictions. This may include techniques such as ensemble averaging or majority voting if multiple random forest models or other machine learning algorithms are employed in parallel.

The system may generate a detailed report as part of the classification output step 1314. This report may include the final classification, associated confidence scores, and relevant feature importance information. The report may also present visualizations such as receiver operating characteristic (ROC) curves or confusion matrices to illustrate the model's performance.

In some cases, the classification output step 1314 may incorporate a decision support system to assist clinicians in interpreting the results. This system may provide recommendations for further testing or treatment based on the classification output and other clinical factors.

The classification output step 1314 may also include a mechanism for flagging uncertain or borderline cases that may require additional review or testing. This may involve setting secondary thresholds or using clustering techniques to identify samples that fall in ambiguous regions of the decision space.

In some implementations, the classification output step 1314 may include a temporal analysis component, comparing the current classification result with previous results for the same patient to track changes over time. This longitudinal perspective may provide additional context for clinical decision-making.

The system may also incorporate a feedback loop as part of the classification output step 1314, allowing clinicians to provide input on the accuracy of the classifications. This feedback may be used to continuously refine and improve the performance of the random forest model and the overall diagnostic system.

The system 1300 incorporates both genetic analysis and machine learning components to process biological sample data. The arrangement of steps shows how the system processes information from initial sample collection to final diagnostic output.

The methods and systems disclosed may provide several technological improvements in the field of lung allograft dysfunction diagnosis:

1. Enhanced diagnostic accuracy: By combining the AI2 gene set with cell type-specific genes in a random forest machine learning model, the system may achieve higher accuracy in distinguishing between CLAD, ALAD, and control samples compared to traditional diagnostic methods.

2. Improved data integration: The system may effectively integrate multiple types of genetic information, including the AI2 gene set and cell type-specific genes, providing a more comprehensive analysis of lung allograft health.

3. Robust feature selection: The random forest model's feature importance analysis may identify the most informative genes and cell type markers for diagnosis, leading to more targeted and efficient diagnostic tests in the future.

4. Adaptive learning capabilities: The incorporation of a feedback loop in the classification output step may allow the system to continuously improve its performance over time based on clinician input and new data.

5. Personalized medicine approach: By including temporal analysis and tracking changes in individual patients over time, the system may support more personalized and precise diagnostic and treatment strategies.

6. Handling of complex data: The random forest model's ability to process high-dimensional data and handle non-linear relationships may allow for more sophisticated analysis of gene expression patterns in lung allograft dysfunction.

7. Improved early detection: The system's ability to detect subtle changes in gene expression patterns may enable earlier diagnosis of CLAD or ALAD, leading to more timely interventions and improved patient outcomes.

8. Standardization of diagnosis: By providing a systematic, data-driven approach to lung allograft dysfunction diagnosis, the system may help reduce variability in diagnostic practices across different clinical settings.

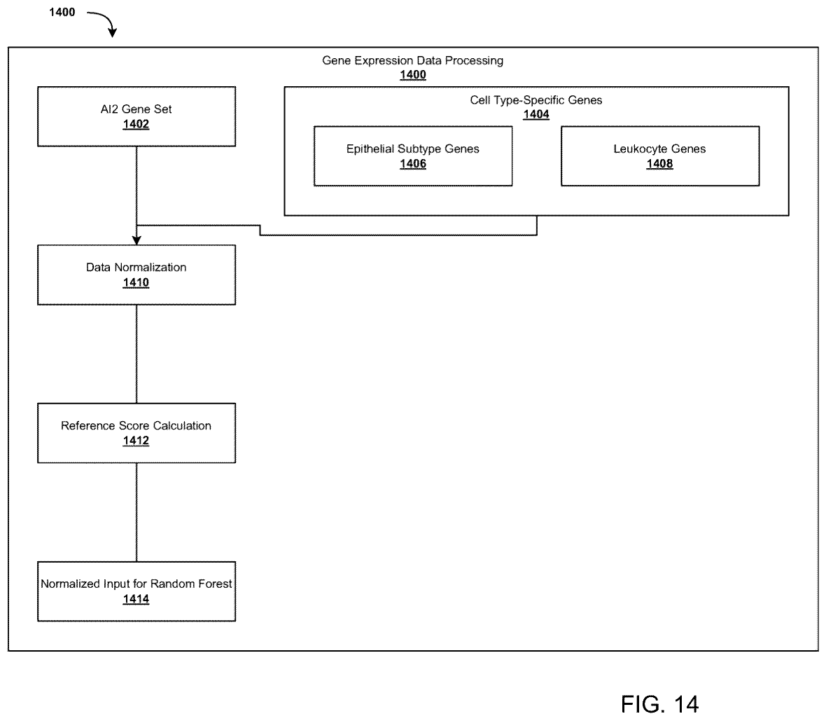

FIG. 14 illustrates a flowchart of a gene expression data processing method 1400. The method 1400 includes two parallel input paths that converge into a sequential processing sequence.

The first input path begins with an AI2 gene set 1402. The second input path comprises cell type-specific genes 1404, which includes two subcomponents: epithelial subtype genes 1406 and leukocyte genes 1408.

The first input path in the gene expression data processing method 1400 begins with the AI2 gene set 1402. This gene set may comprise a collection of genes identified as relevant markers for assessing lung allograft health and function. In some implementations, the AI2 gene set may include genes associated with immune response and inflammation in the context of lung transplantation.

The AI2 gene set 1402 may serve as a primary input for the subsequent processing steps. In some cases, the expression levels of these genes may be quantified using clinically applicable sequencing technologies, such as quantitative PCR, reverse transcription-loop mediated isothermal amplification (LAMP), or digital RNA counting methods.

The gene expression data from the AI2 gene set 1402 may undergo preprocessing before entering the main processing sequence. This preprocessing may involve steps such as quality control to identify and potentially exclude outliers or samples with poor sequencing quality. In some implementations, the preprocessing may include normalization techniques to account for technical variations in sample preparation and sequencing depth.

The AI2 gene set data may then flow into a data normalization step 1410. This step may involve various normalization methods to make the gene expression data comparable across different samples and experimental conditions. In some cases, the normalization process may include techniques such as quantile normalization, scaling to a reference gene set, or applying a logarithmic transformation to address the skewed distribution often observed in gene expression data.

Following normalization, the AI2 gene set data may proceed to subsequent steps in the processing sequence, where it may be combined with data from the cell type-specific genes path for further analysis and classification. This integration of the AI2 gene set with other genetic information may contribute to a more comprehensive assessment of lung allograft dysfunction.

The second input path in the gene expression data processing method 1400 begins with the cell type-specific genes 1404. This component may include genes associated with various cell types present in airway brush samples, providing information about the cellular composition of the tissue.

The cell type-specific genes 1404 may be further divided into two subcomponents: epithelial subtype genes 1406 and leukocyte genes 1408. This division may allow for a more nuanced analysis of the cellular landscape in lung allograft samples.

The epithelial subtype genes 1406 may include markers for different types of epithelial cells found in the airway. In some implementations, these genes may comprise markers such as SCGB3A1 for secretory or club cells, MS4A8 for ciliated cells, KRT5 for basal cells, and CALM1 for transitional epithelial cells. The expression levels of these genes may provide insights into the relative abundance and health of various epithelial cell populations in the sample.

The leukocyte genes 1408 may include markers associated with different types of immune cells. In some cases, these genes may include PTPRC (CD45) as a general leukocyte marker, MARCO for macrophages, and GNLY for certain T cell and natural killer cell subsets. The expression patterns of these genes may offer information about the immune cell composition and potential inflammatory processes in the lung allograft.

The gene expression data for the cell type-specific genes may be obtained using similar sequencing technologies as those used for the AI2 gene set, such as quantitative PCR, reverse transcription-loop mediated isothermal amplification (LAMP), or digital RNA counting methods. In some implementations, the data from both epithelial subtype genes and leukocyte genes may undergo preprocessing steps, including quality control and initial normalization.

The processed data from the cell type-specific genes path may then be integrated with the normalized AI2 gene set data in subsequent steps of the method 1400. This integration may occur during the reference score calculation step 1412 or as part of the input preparation for the random forest model in step 1414.

By incorporating cell type-specific gene expression data alongside the AI2 gene set, the method 1400 may account for variations in cellular composition across different samples. This approach may enhance the accuracy and robustness of the lung allograft dysfunction diagnosis by providing a more comprehensive view of the tissue microenvironment.

From the AI2 gene set 1402, the method 1400 proceeds to a data normalization step 1410.

The data normalization step 1410 may involve several techniques to standardize the gene expression data from the AI2 gene set and prepare it for subsequent analysis. In some implementations, this step may include quantile normalization, which may adjust the distribution of gene expression values across samples to follow a common distribution. This approach may help reduce technical variability between samples while preserving biological differences.

In some cases, the normalization process may involve scaling the gene expression data to a set of reference genes. This method may use genes known to have stable expression levels across different conditions as a baseline for normalizing the expression of other genes. The choice of reference genes may be based on prior knowledge or determined through statistical analysis of the dataset.

The data normalization step 1410 may also incorporate a log transformation of the gene expression values. This transformation may help address the skewed distribution often observed in gene expression data and may make the data more suitable for downstream analysis. In some implementations, a log 2 transformation may be applied, while in others, a natural log or log 10 transformation may be used depending on the specific requirements of the analysis pipeline.

In some aspects, the normalization step may include methods to account for batch effects or other sources of technical variation. This may involve the use of techniques such as ComBat or surrogate variable analysis (SVA) to identify and remove unwanted variation while preserving biological signal.

The data normalization step 1410 may also involve handling missing or zero values in the gene expression data. In some cases, imputation methods may be used to estimate missing values, while in others, genes with a high proportion of zero or missing values across samples may be filtered out.

In some implementations, the normalization process may include adjusting for differences in sequencing depth or library size between samples. This may involve techniques such as reads per kilobase of transcript per million mapped reads (RPKM) or transcripts per million (TPM) normalization, which may account for differences in both gene length and sequencing depth.

The output of the data normalization step 1410 may be a set of normalized gene expression values that are comparable across samples and suitable for input into subsequent analysis steps, such as the reference score calculation or the random forest model.

Following normalization, the method 1400 continues to a reference score calculation step 1412.

After the reference score calculation, the method 1400 moves to a normalized input for random forest step 1414. This step 1414 processes the normalized data for use in a random forest machine learning model.

The normalized input for random forest step 1414 may involve several processes to prepare the normalized gene expression data for input into the random forest machine learning model. In some implementations, this step may combine the normalized AI2 gene set data with the processed cell type-specific gene data to create a comprehensive feature set for the model.

The step 1414 may include feature scaling techniques to ensure all input variables are on a similar scale. This may involve methods such as min-max scaling or standardization, which may help prevent features with larger magnitudes from dominating the model's decision-making process.

In some cases, the step 1414 may incorporate feature selection or dimensionality reduction techniques. This may include methods such as principal component analysis (PCA) or recursive feature elimination to identify the most informative features for the random forest model. These techniques may help reduce noise in the data and improve the model's performance.

The normalized input preparation may also involve handling any remaining missing values or outliers in the dataset. In some implementations, this may include imputation techniques for missing data or methods to detect and address extreme values that could potentially skew the model's results.

Step 1414 may also include data formatting to ensure compatibility with the specific implementation of the random forest algorithm being used. This may involve organizing the normalized gene expression data into appropriate data structures or file formats accepted by the machine learning framework.

In some aspects, the step 1414 may incorporate data augmentation techniques to increase the diversity of the training set. This may include methods such as adding small amounts of noise to the gene expression values or generating synthetic samples using techniques like SMOTE (Synthetic Minority Over-sampling Technique), particularly if dealing with imbalanced datasets.

The output of step 1414 may be a prepared dataset ready for input into the random forest model, with features appropriately scaled, selected, and formatted to maximize the model's ability to learn patterns associated with lung allograft dysfunction.

The flowchart shows how the gene expression data flows through various processing stages, with the cell type-specific genes 1404 connecting to the main processing path after the AI2 gene set 1402. The arrangement of steps indicates a systematic approach to preparing gene expression data for analysis through a random forest model.

The methods and systems may represent several technological improvements over conventional approaches to diagnosing lung allograft dysfunction:

1. Enhanced data integration: The method may combine multiple types of gene expression data, including the AI2 gene set and cell type-specific genes, into a unified analysis framework. This integrated approach may provide a more comprehensive view of the lung allograft microenvironment compared to methods that focus on a single gene set or cell type.

2. Improved normalization techniques: The data normalization step may incorporate advanced methods such as quantile normalization, reference gene scaling, and batch effect correction. These techniques may help reduce technical variability and enhance the biological signal in the gene expression data, potentially leading to more accurate diagnostic results.

3. Adaptive feature selection: The use of a random forest model with feature importance analysis may allow for dynamic selection of the most informative genes for diagnosis. This approach may adapt to individual patient characteristics and evolving disease patterns more effectively than static gene panels.

4. Robust handling of heterogeneous data: By incorporating cell type-specific genes, the method may account for variations in cellular composition across samples. This may improve the reliability of the diagnosis in cases where cellular heterogeneity could confound traditional analysis methods.

5. Scalable machine learning integration: The use of a random forest model may allow for efficient processing of high-dimensional gene expression data. This scalable approach may handle larger datasets and more complex gene interactions compared to simpler statistical methods.

6. Flexible data preprocessing: The method may accommodate various types of gene expression data, including those from different sequencing technologies. This flexibility may facilitate the integration of data from multiple sources or time points, potentially enabling more comprehensive longitudinal analyses.

7. Enhanced outlier detection: The preprocessing and normalization steps may include advanced methods for identifying and handling outliers and missing data. This may improve the overall data quality and reduce the impact of technical artifacts on the diagnostic results.

8. Improved interpretability: By combining machine learning with biologically relevant gene sets, the method may provide results that are both accurate and interpretable in a clinical context. This may enhance the utility of the diagnostic tool for healthcare providers.

9. Personalized medicine: The integration of cell type-specific data with the AI2 gene set may allow for more nuanced patient stratification. This approach may support the development of personalized treatment strategies based on individual gene expression profiles.

10. Streamlined clinical implementation: The use of clinically applicable sequencing technologies and the ability to work with unnormalized counts may facilitate the translation of this method into clinical practice. This may reduce the barriers to adoption compared to more complex or specialized laboratory techniques.

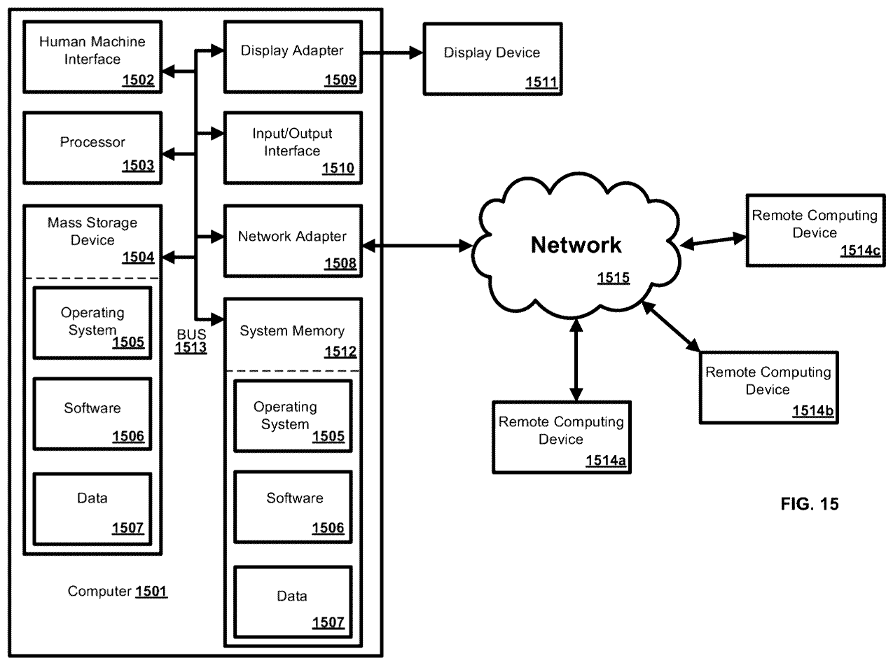

In an exemplary aspect, the methods and systems can be implemented on a computer 1501 as illustrated in FIG. 15 and described below. Similarly, the methods and systems disclosed can utilize one or more computers to perform one or more functions in one or more locations. FIG. 15 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 1501. The components of the computer 1501 can comprise, but are not limited to, one or more processors 1503, a system memory 1512, and a system bus 1513 that couples various system components including the one or more processors 1503 to the system memory 1512. The system can utilize parallel computing.

The system bus 1513 represents one or more of several possible types of bus structures, including a memory bus or memory controller, a peripheral bus, an accelerated graphics port, or local bus using any of a variety of bus architectures. The bus 1513, and all buses specified in this description can also be implemented over a wired or wireless network connection and each of the subsystems, including the one or more processors 1503, a mass storage device 1504, an operating system 1505, software 1506, data 1507, a network adapter 1508, the system memory 1512, an Input/Output Interface 1510, a display adapter 1509, a display device 1511, and a human machine interface 1502, can be contained within one or more remote computing devices 1514a,b,c at physically separate locations, connected through buses of this form, in effect implementing a fully distributed system.

The computer 1501 typically comprises a variety of computer readable media. Exemplary readable media can be any available media that is accessible by the computer 1501 and comprises, for example and not meant to be limiting, both volatile and non-volatile media, removable and non-removable media. The system memory 1512 comprises computer readable media in the form of volatile memory, such as random access memory (RAM), and/or non-volatile memory, such as read only memory (ROM). The system memory 1512 typically contains data such as the data 1507 and/or program modules such as the operating system 1505 and the software 1506 that are immediately accessible to and/or are presently operated on by the one or more processors 1503.

In another aspect, the computer 1501 can also comprise other removable/non-removable, volatile/non-volatile computer storage media. By way of example, FIG. 15 illustrates the mass storage device 1504 which can provide non-volatile storage of computer code, computer readable instructions, data structures, program modules, and other data for the computer 1501. For example and not meant to be limiting, the mass storage device 1504 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 1504, including by way of example, the operating system 1505 and the software 1506. Each of the operating system 1505 and the software 1506 (or some combination thereof) can comprise elements of the programming and the software 1506. The data 1507, such as the AI2 gene set and/or other gene sets, can also be stored on the mass storage device 1504. The data 1507 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In an aspect, the software 1506 can comprise artificial intelligence software, such a software configured for implementing a random forest machine learning algorithm.

In another aspect, the user can enter commands and information into the computer 1501 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the one or more processors 1503 via the human machine interface 1502 that is coupled to the system bus 1513, but can be connected by other interface and bus structures, such as a parallel port, game port, an IEEE 1394 Port (also known as a Firewire port), a serial port, or a universal serial bus (USB).

In yet another aspect, the display device 1511 can also be connected to the system bus 1513 via an interface, such as the display adapter 1509. It is contemplated that the computer 1501 can have more than one display adapter 1509 and the computer 1501 can have more than one display device 1511. For example, the display device 1511 can be a monitor, an LCD (Liquid Crystal Display), or a projector. In addition to the display device 1511, other output peripheral devices can comprise components such as speakers (not shown) and a printer (not shown) which can be connected to the computer 1501 via the Input/Output Interface 1510. Any step and/or result of the methods can be output in any form to an output device. Such output can be any form of visual representation, including, but not limited to, textual, graphical, animation, audio, tactile, and the like. The display device 1511 and computer 1501 can be part of one device, or separate devices.

The computer 1501 can operate in a networked environment using logical connections to one or more remote computing devices 1514a,b,c. By way of example, a remote computing device can be a personal computer, portable computer, smartphone, a server, a router, a network computer, a peer device or other common network node, and so on. Logical connections between the computer 1501 and a remote computing device 1514a,b,c can be made via a network 1515, such as a local area network (LAN) and/or a general wide area network (WAN). Such network connections can be through the network adapter 1508. The network adapter 1508 can be implemented in both wired and wireless environments.

For purposes of illustration, application programs and other executable program components such as the operating system 1505 are illustrated herein as discrete blocks, although it is recognized that such programs and components reside at various times in different storage components of the computing device 1501, and are executed by the one or more processors 1503 of the computer. An implementation of the software 1506 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

### EXAMPLES

### A. Example 1: Small Airway Brush Gene Expression Predicts Chronic Lung Allograft Dysfunction and Mortality

Candidate genes from 4 rejection-associated transcript sets were assessed in 156 small airway brushes in a derivation cohort. Brushes were serially collected before CLAD and during early or late CLAD in 45 recipients and in 37 time-matched controls with >1-year stable lung function. Multivariable CLAD models were GEE-adjusted for repeat measures. Time to graft failure (death with CLAD or retransplant) was assessed with Cox models. Candidate genes not associated with CLAD or time to graft failure were excluded, yielding the Airway Inflammation 2 (AI2) gene set. Area under receiver operating curve (AUC) for CLAD and competing risks of death or graft failure were assessed in an independent validation cohort of 37 CLAD cases and 37 controls.

The 4 candidate gene sets identified CLAD in brushes taken before CLAD onset. Thirty-two genes were associated with CLAD and graft failure, comprising the AI2 score, which clustered into three subcomponents. The AI2 score identified CLAD in derivation and validation cohorts (AUC 0.74-0.89). AI2 score also predicted time to graft failure and all-cause mortality in both cohorts (P<0.03).

These results indicate that airway inflammation transcripts are linked to CLAD, subsequent graft survival, and overall mortality.

### Methods

### Derivation Cohort

Lung transplant recipients were prospectively enrolled in a longitudinal cohort that included optional airway brushing for research. Starting at 2 months post-transplant, participants underwent airway brushing during surveillance and clinically indicated bronchoscopies whenever transbronchial biopsies were performed. Airway brushes were performed between 2015 and 2022 in recipients transplanted between 2004 and 2020. A 2-mm cytology brush (Conmed #129) was advanced under fluoroscopic guidance to 1-2 cm from the periphery, inserted and retracted 10 times, and placed into a QIAzol (#79306, QIAGEN, Germantown, MD) tube in the bronchoscopy suite. The tubes were vortexed to dislodge genomic material, and brushes were discarded before storage of the lysates at −80° C.

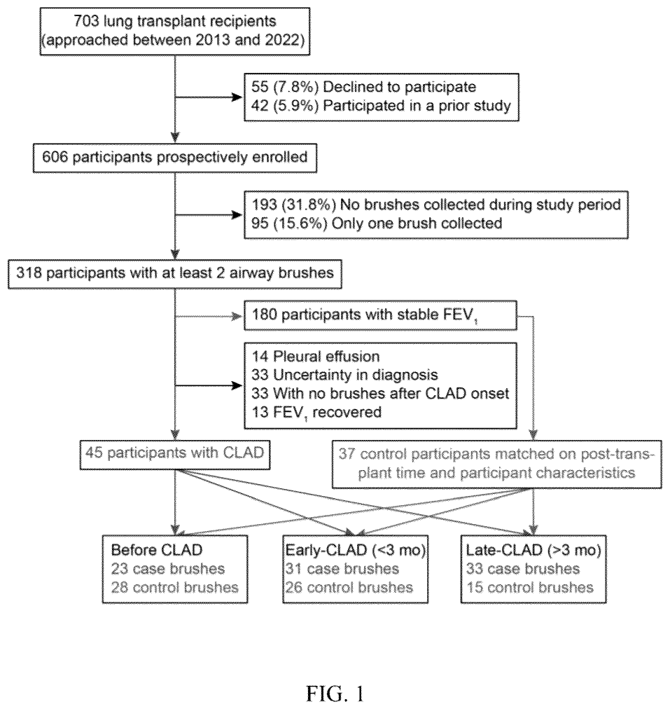

Participants were considered for inclusion if they received at least 2 airway brushings, taken before and after CLAD onset, and were not previously included in our prior study of the lymphocytic bronchitis metagene as a predictor of CLAD (FIG. 1) (8). CLAD was defined per 2019 International Society for Heart and Lung Transplant (ISHLT) guidelines by a persistent >20% decline in FEV1 from post-transplant baseline, absent alternative explanations for this decline, as adjudicated by 2 investigators (1). Control participants with over 1-year of stable lung function their first brush were matched from potential candidates targeting a 1:1 ratio by minimizing the sum of the square of the standardized mean difference in donor and recipient age, sex, ethnicity, body mass index, transplant indication, CMV serostatus, and time post-transplant. Control participants were also manually adjudicated. Brushes were grouped into before CLAD, early CLAD (within 3 months of CLAD onset), and late CLAD (>3 months from CLAD onset) groups, with each participant contributing no more than one brush per time group. Some participants had only one brush included, while others had 2 or 3.

Time to graft failure was defined as the interval between airway brush and retransplantation or death with CLAD. Non-CLAD causes of death, like malignancy or heart failure, were considered separately in competing risks models but included in all-cause mortality models. Infection was assessed based on clinical microbiology detection of one or more of a pre-determined list of likely pathogens in concomitant BAL fluid.

### Validation Cohort

Airway brushings were performed in consenting lung transplant recipients. Participants were prospectively identified based on clinical diagnosis of CLAD or as non-CLAD control participants with stable lung function. CLAD diagnosis followed 2019 ISHLT guidelines, like the derivation cohort (1). Cases in the validation cohort had CLAD at the time of brushing, like the UCSF early or late CLAD groups. In some cases, small airway brushing was performed where brushes were cut into RLT Plus buffer (Qiagen #1053393) prior to storage.

### RNA Sequencing

RNA was extracted from small airway brushings using Qiagen miRNeasy Mini Kit (UCSF) or Qiagen RNeasy kit (Pitt), as previously described in Dugger D T et al., Chronic lung allograft dysfunction small airways reveal a lymphocytic inflammation gene signature. Am J Transplant 2021; 21: 362-371 and Iasella C J et al., Type-1 immunity and endogenous immune regulators predominate in the airway transcriptome during chronic lung allograft dysfunction. Am J Transplant 2021; 21: 2145-2160 (8, 9). At UCSF, libraries were prepared using the NEBNext Ultra2 kit (New England Biolabs, Ipswich, MA) on a Labcyte Echo (Beckman Coulter, Pasadena, CA). Depletion of rRNA was carried out by using the Qiagen FastSelect approach (UCSF). Initial low depth sequencing was conducted in a pool including an equal volume of library RNA for each sample to normalize reads for a high depth NovaSeq (Illumina, San Diego, CA) run. Pitt libraries were prepared using Illumina reagents, also with rRNA depletion. Reads were aligned to the hg38 human genome using STAR (UCSF) and CLC Genomics Workbench (Pitt). HLA typing was imputed with arcasHLA and matched to UNOS data to confirm sample identities (11).

Aligned RNAseq gene counts were normalized using DESeq2 and transformed with the “vst” function. Metagene scores were calculated as the sum of target gene counts centered and scaled to a mean of 0 and SD 1.

### Statistical Analysis

Participant characteristics in derivation and validation cohorts were compared across strata of CLAD status by chi-square test for categorical variables, Wilcox test for non-parametric variables, and t-test for normally distributed variables, as determined by the Shapiro-Wilk test. FEV1 slope was calculated exponentially weighted moving average of spirometry data over time.

Differences in metagene scores were compared by Wilcoxon rank sum test. To derive the airway inflammation score 2 (AI2) score, genes from UCSF's lymphocytic bronchitis score (12), Pitt's CLAD score (9), the Renal Rejection versus Everything Else score (13, 14), and the Common Rejection Module (15) were assessed. Genes not meeting the following criteria were excluded: 1) positive association with CLAD in a univariable logistic regression model using generalized estimating equations to adjust for multiple comparisons; and 2) positive association with time to graft failure in a Cox hazards model. Because negative associations were not distinguished from no association, a one-tailed alpha of 0.05 threshold was used. Co-expression of AI2 genes was assessed by hierarchical clustering using a complete linkage function of Pearson correlations.

The XGBoost algorithm was used with leave-one-out cross validation to develop an unbiased comparator classifier, with default hyperparameters (eta learning rate 0.3, gamma 0, lambda regularization 1, alpha 0, maximum depth 6, child weight 1, and no subsampling). Classifier performance characteristics were visualized by receiving operating curve (ROC). Area under the ROC (AUC) 95% confidence intervals were determined using the DeLong Method.

Comparisons across CLAD strata were performed by Mann-Whitney test within time groups or using GEE-adjusted models with an exchangeable covariance matrix where there were repeat measures across individuals. A multivariable Cox regression model was employed to assess the association between AI2 score and times to graft failure or all-cause mortality. Graft failure time to event models included three outcome states: censor, retransplant or death from CLAD, or death not attributed to CLAD. Multivariable Cox regression models for graft and patient survival included donor and recipient age, sex, and self-reported White ethnicity, transplant type (single versus double) and UNOS group (A, B or C versus D), donor-specific antibodies (DSA), acute cellular rejection (ACR, ISHLT grade A1 or B1R or greater), and positive bronchoalveolar lavage (BAL) cultures or viral studies as covariates. ACR, DSA, and positive BAL microbial studies were calculated as binary variables referring to the recipient status at the time of bronchoscopy when airway brushing was performed. To determine if the AI2 score provided prognostic data within individuals diagnosed with CLAD, pooled Cox analyses of to time to graft failure (death with CLAD or retransplantation) and retransplant-free survival (including death from non-CLAD causes) was performed, including only CLAD cases from the derivation and validation cohorts, using study center as a covariate. AI2 score was assessed as a continuous variable in Cox models but shown graphically stratified by tertiles.

### Results:

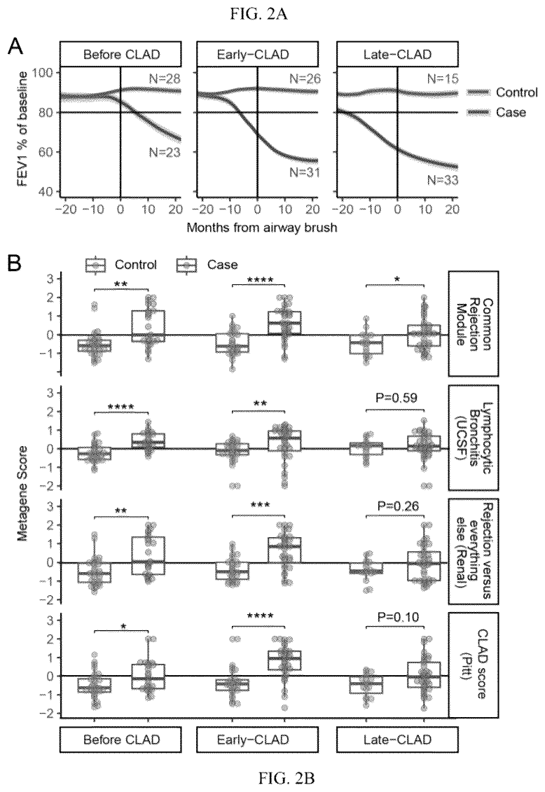

Over 92 percent of potential participants consented to airway brushing for research purposes and over a 9-year study period two or more serial airway brushes were obtained from 318 subjects in the derivation cohort. There were no brushing-related adverse events. As shown in FIG. 1, 156 airway brushes were included in the study from 45 CLAD-participants and 37 matched control participants with stable lung function. Eight control participants contributed only one brush. Multiple brushes were included for 60 participants, grouped as: before CLAD, early-CLAD, and late-CLAD or at matched time-points. Only a single brush for 14 cases and 8 controls so that each participant only contributed up to 1 brush per time point. Five samples failed quality control metrics (3%). As shown in FIG. 2A, for CLAD cases in the before CLAD time point, the FEV1 had not yet crossed the 20% threshold, while FEV1 decline was steepest in the early-CLAD group. Aggregated derivation cohort participant characteristics are shown in Table 2, and participant characteristics for each time grouping are seen in Table 3. Before CLAD brushes were collected at a median 1.6 years from transplant, early CLAD brushes at 2.3 years, and late CLAD brushes at 4.7 years. Late CLAD bronchoscopies were less commonly for surveillance, and those participants were more likely to have had an mTOR inhibitor added to their immune suppression regimen.

TABLE 2
Derivation Cohort (UCSF) Participant Characteristics

| | Control | | Case | | p-value |
|---|---|---|---|---|---|
| N | 37 | | 45 | | |
| Recipient Age, median [IQR] | 59 | [50, 67] | 57 | [50, 64 | 0.31 |
| Donor Age, median [IQR] | 40 | [21, 48] | 33 | [23, 49] | 0.93 |
| Male Recipient, N (%) | 21 | (56.8) | 29 | (64.4) | 0.63 |
| Male Donor, N (%) | 23 | (62.2) | 31 | (68.9) | 0.69 |
| UNOS Transplant Indication Group, N (%) | | | | | 0.65 |
| A-Obstructive | 3 | (8.1) | 8 | (17.8) | |
| B-Pulmonary vascular | 1 | (2.7) | 1 | (2.2) | |
| C-Cystic fibrosis | 3 | (8.1) | 3 | (6.7) | |
| D-Restrictive | 30 | (81.1) | 33 | (73.3) | |
| Double lung transplant, N (%) | 34 | (91.9) | 43 | (95.6) | 0.82 |
| Cytomegalovirus serostatus, N (%) | | | | | 0.12 |
| Donor+, Recipient− | 11 | (29.7) | 8 | (17.8) | |
| Donor+, Recipient+ | 9 | (24.3) | 20 | (44.4) | |
| Donor−, Recipient+ | 13 | (35.1) | 12 | (26.7) | |
| Donor−, Recipient− | 2 | (5.4) | 5 | (11.1) | |
| Recipient Ethnicity, N (%) | | | | | 0.36 |
| White | 26 | (70.3) | 30 | (66.7) | |
| Black | 3 | (8.1) | 5 | (11.1) | |
| Hispanic | 6 | (16.2) | 8 | (17.8) | |
| Asian | 2 | (5.4) | 0 | (0.0) | |
| Multiracial | 0 | (0) | 2 | (4.4) | |
| Donor Ethnicity, N (%) | | | | | 0.60 |
| White | 19 | (51.4) | 23 | (51.1) | |
| Black | 3 | (8.1) | 4 | (8.9) | |
| Hispanic | 12 | (32.4) | 12 | (26.7) | |
| Asian | 2 | (5.4) | 6 | (13.3) | |
| Native Hawaiian/other Pacific | 1 | (2.7) | 0 | (0.0) | |
| Islander | | | | | |

TABLE 3
Derivation Cohort Participant Characteristics by Time point.

| | | Before CLAD | | | | Early CLAD | | | | Late CLAD | | | | |
|---|---|---|---|---|---|---|---|---|---|---|---|---|---|---|
| | | Control | | Case | | Control | | Case | | Control | | Case | | p-value |
| N | | 28 | | 23 | | 26 | | 31 | | 15 | | 33 | | |
| Recipient Age, | | 58 | [47, 66] | 57 | [50, 64] | 59 | [53, 67] | 55 | [50, 64] | 62 | [50, 66] | 57 | [50, 64] | 0.84 |
| median [IQR] | | | | | | | | | | | | | | |
| Donor Age, | | 42 | [23, 50] | 33 | [26, 50] | 30 | [20, 47] | 33 | [25, 5] | 24 | [21, 43] | 39 | [26, 49] | 0.77 |
| median [IQR] | | | | | | | | | | | | | | |
| Male Recipient, | | 15 | (53.6) | 13 | (56.5) | 14 | (53.8) | 18 | (58.1) | 9 | (60.0) | 25 | (75.8) | 0.48 |
| N (%) | | | | | | | | | | | | | | |
| Male Donor, | | 19 | (67.9) | 16 | (69.6) | 17 | (65.4) | 20 | (64.5) | 8 | (53.3) | 22 | (66.7) | 0.94 |
| N (%) | | | | | | | | | | | | | | |
| UNOS Transplant Indication Group, N (%) | | | | | | | | | | | | | | 0.86 |
| | A-Obstructive | 2 | (7.1) | 1 | (4.3) | 2 | (7.7) | 5 | (16.1) | 1 | (6.7) | 7 | (21.2) | |
| | B-Pulmonary | 1 | (3.6) | 1 | (4.3) | 1 | (3.8) | 1 | (3.2) | 1 | (6.7) | 0 | (0.0) | |
| | vascular | | | | | | | | | | | | | |
| | C-Cystic | 3 | (10.7) | 2 | (8.7) | 2 | (7.7) | 2 | (6.5) | 0 | (0.0) | 2 | (6.1) | |
| | fibrosis | | | | | | | | | | | | | |
| | D-Restrictive | 22 | (78.6) | 19 | (82.6) | 21 | (80.8) | 23 | (74.2) | 13 | (86.7) | 24 | (72.7) | |
| Cytomegalovirus serostatus, N (%) | | | | | | | | | | | | | | 0.19 |
| | Donor+, | 8 | (28.6) | 6 | (26.1) | 7 | (26.9) | 7 | (22.6) | 7 | (46.7) | 5 | (15.2) | |
| | Recipient− | | | | | | | | | | | | | |
| | Donor+, | 7 | (25.0) | 9 | (39.1) | 7 | (26.9) | 14 | (45.2) | 2 | (13.3) | 15 | (45.5) | |
| | Recipient+ | | | | | | | | | | | | | |
| | Donor−, | 11 | (39.3) | 7 | (30.4) | 8 | (30.8) | 7 | (22.6) | 3 | (20.0) | 9 | (27.3) | |
| | Recipient+ | | | | | | | | | | | | | |
| | Donor−, | 2 | (7.1) | 1 | (4.3) | 2 | (7.7) | 3 | (9.7) | 1 | (6.7) | 4 | (12.1) | |
| | Recipient− | | | | | | | | | | | | | |
| Recipient Ethnicity, N (%) | | | | | | | | | | | | | | 0.91 |
| | White | 19 | (67.9) | 15 | (65.2) | 18 | (69.2) | 18 | (58.1) | 10 | (66.7) | 22 | (66.7) | |
| | Black | 2 | (7.1) | 2 | (8.7) | 2 | (7.7) | 5 | (16.1) | 1 | (6.7) | 3 | (9.1) | |
| | Hispanic | 5 | (17.9) | 5 | (21.7) | 5 | (19.2) | 6 | (19.4) | 3 | (20.0) | 6 | (18.2) | |
| | Asian | 2 | (7.1) | 0 | (0.0) | 1 | (3.8) | 0 | (0.0) | 1 | (6.7) | 0 | (0.0) | |
| | Multiracial | 0 | (0.0) | 1 | (4.3) | 0 | (0.0) | 2 | (6.5) | 0 | (0.0) | 2 | (6.1) | |
| Donor Ethnicity, N (%) | | | | | | | | | | | | | | 0.96 |
| | White | 15 | (53.6) | 11 | (47.8) | 14 | (53.8) | 16 | (51.6) | 6 | (40.0) | 15 | (45.5) | |
| | Black | 1 | (3.6) | 3 | (13.0) | 3 | (11.5) | 4 | (12.9) | 2 | (13.3) | 3 | (9.1) | |
| | Hispanic | 10 | (35.7) | 6 | (26.1) | 7 | (26.9) | 7 | (22.6) | 5 | (33.3) | 10 | (30.3) | |
| | Asian | 1 | (3.6) | 3 | (13.0) | 2 | (7.7) | 4 | (12.9) | 2 | (13.3) | 5 | (15.2) | |
| | Native Hawaiian/ | 1 | (3.6) | 0 | (0.0) | 0 | (0.0) | 0 | (0.0) | 0 | (0.0) | 0 | (0.0) | |
| | other Pacific | | | | | | | | | | | | | |
| | Islander | | | | | | | | | | | | | |
| FEV₁ slope | | 10.6 | (19.0) | −11.4 | (44.3) | 0.8 | (10.5) | −75.4 | (53.1) | −0.6 | (16.2) | −34.5 | (43.7) | <0.01 |
| (ml/month), | | | | | | | | | | | | | | |
| mean (SD) | | | | | | | | | | | | | | |
| A-grade | | | | | | | | | | | | | | 0.14 |
| rejection, N (%) | | | | | | | | | | | | | | |
| | 0 | 28 | (100) | 21 | (91.3) | 26 | (100) | 27 | (87.1) | 14 | (93.3) | 32 | (97) | |
| | 1 | 0 | (0) | 0 | (0) | 0 | (0) | 2 | (6.5) | 0 | (0) | 1 | (3) | |
| | 2 | 0 | (0) | 2 | (8.7) | 0 | (0) | 1 | (3.2) | 0 | (0) | 0 | (0) | |
| B-grade | | | | | | | | | | | | | | 0.16 |
| rejection, N (%) | | | | | | | | | | | | | | |
| | 0 | 28 | (100) | 22 | (95.7) | 25 | (96.2) | 25 | (80.6) | 14 | (93.3) | 3 | (93.9) | |
| | 1R | 0 | (0) | 1 | (4.3) | 1 | (3.8) | 5 | (16.1) | 0 | (0.0) | 1 | (3.0) | |

BAL microbiology culture positive, N (%)

| | | | | | | | | | | | | | |
|---|---|---|---|---|---|---|---|---|---|---|---|---|---|
| Bacteria | 7 | (25.0) | 5 | (21.7) | 8 | (30.8) | 7 | (22.6) | 2 | (13.3) | 8 | (24.2) | 0.89 |
| Virus | 1 | (3.6) | 5 | (21.7) | 0 | (0.0) | 2 | (6.5) | 0 | (0.0) | 2 | (6.1) | 0.03 |
| Fungus | 2 | (7.1) | 3 | (13.0) | 0 | (0.0) | 4 | (12.9) | 1 | (6.7) | 4 | (12.1) | 0.52 |
| Acid Fast | 0 | (0.0) | 0 | (0.0) | 0 | (0.0) | 1 | (3.2) | 0 | (0.0) | 0 | (0.0) | 0.54 |
| Bacilli | | | | | | | | | | | | | |

| | | | | | | | | | | | | | |
|---|---|---|---|---|---|---|---|---|---|---|---|---|---|
| Years from | 1.5 | [1.1, 1.8] | 1.7 | [0.8, 2.4] | 2.2 | [1.7, 2.4] | 4.4 | [1.8, 6.8] | 4.1 | [2.3, 6.2] | 5.2 | [2.3, 7.8] | <0.01 |
| transplant to brush, | | | | | | | | | | | | | |
| median [IQR] | | | | | | | | | | | | | |
| Months without | 54 | [48, 66] | 11 | [3, 24] | 48 | [36, 52] | −0.2 | [−2, 0] | 42 | [15, 48] | −12 | [−22, −4] | <0.01 |
| CLAD from brush, | | | | | | | | | | | | | |
| median [IQR] | | | | | | | | | | | | | |

Analyses in FIGS. 1-5 were limited to the derivation cohort. Participant characteristics for the 37 cases and 37 controls in the validation cohort are shown in Table 4. Validation cohort cases were similar to early CLAD cases in the derivation cohort; however, the frequency of pulmonary fibrosis was less.

TABLE 4
Validation Cohort (Pitt) Participant Characteristics

| | Control | | CLAD | | p-value |
|---|---|---|---|---|---|
| N | 37 | | 37 | | |
| Recipient Age, median | 56 | [37, 64] | 55 | [34, 62] | 0.62 |
| (IQR) | | | | | |
| Male Recipient, N (%) | 19 | (51.4) | 21 | (56.7) | 0.82 |
| Transplant diagnosis, N (%) | | | | | 0.92 |
| COPD | 9 | (24.3) | 8 | (21.6) | |
| CF | 10 | (27.0) | 8 | (21.6) | |
| IPF | 13 | (35.1) | 13 | (35.1) | |
| Redo | 3 | (8.1) | 4 | (10.8) | |
| Other | 2 | (5.4) | 4 | (10.8) | |
| Double-lung transplant, N | 32 | (86.5) | 28 | (75.7) | 0.37 |
| (%) | | | | | |
| Cytomegalovirus serostatus, | | | | | 0.64 |
| N (%) | | | | | |
| Donor+, Recipient− | 8 | (21.6) | 5 | (13.5) | |
| Donor+, Recipient+ | 13 | (35.1) | 18 | (48.6) | |
| Donor−, Recipient+ | 8 | (21.6) | 7 | (18.9) | |
| Donor−, Recipient− | 8 | (21.6) | 7 | (18.9) | |
| Anti-HLA donor-specific | 7 | (18.9) | 2 | (5.4) | 0.15 |
| antibody, N (%) | | | | | |
| Acute rejection, N (%) | 6 | (16.2) | 11 | (29.7) | 0.27 |
| Acute infection, N (%) | 5 | (13.5) | 20 | (54.1) | <0.01 |
| Bacterial | 4 | (10.8) | 14 | (37.8) | 0.01 |
| Viral | 1 | (2.7) | 3 | (8.1) | 0.61 |
| Fungal | 1 | (2.7) | 3 | (8.1) | 0.61 |
| Non-tuberculous | 1 | (2.7) | 3 | (8.1) | 0.61 |
| mycobacterial | | | | | |
| Days to CLAD, median | | | 1310 | [628-2090] | |
| (IQR) | | | | | |
| CLAD stage, N (%) | | | | | |
| 1 | | | 24 | (64.9) | |
| 2 | | | 5 | (13.5) | |
| 3 | | | 8 | (21.6) | |
| 4 | | | 0 | (0) | |

### Candidate CLAD Gene Scores Before and After CLAD Onset:

In the derivation cohort, previously described airway rejection scores (9, 12, 13, 15) were calculated to determine whether they could identify CLAD prior to a 20% decline in FEV1 and for how long this signal would persist. As shown in FIG. 2B, CLAD cases had statistically significant increases in all four of the assessed metagenes at the before CLAD and early CLAD time points. The Lymphocytic Bronchitis score, derived from samples with acute rejection, performed best in before CLAD samples, while the Pitt CLAD score, derived from CLAD airway brushes, performed best in the early CLAD time point. By contrast, in the late-CLAD time point, only the Common Rejection Module gene set had a statistically significantly association with CLAD.

### Selection of Airway Inflammation 2 (AI2) Score Genes

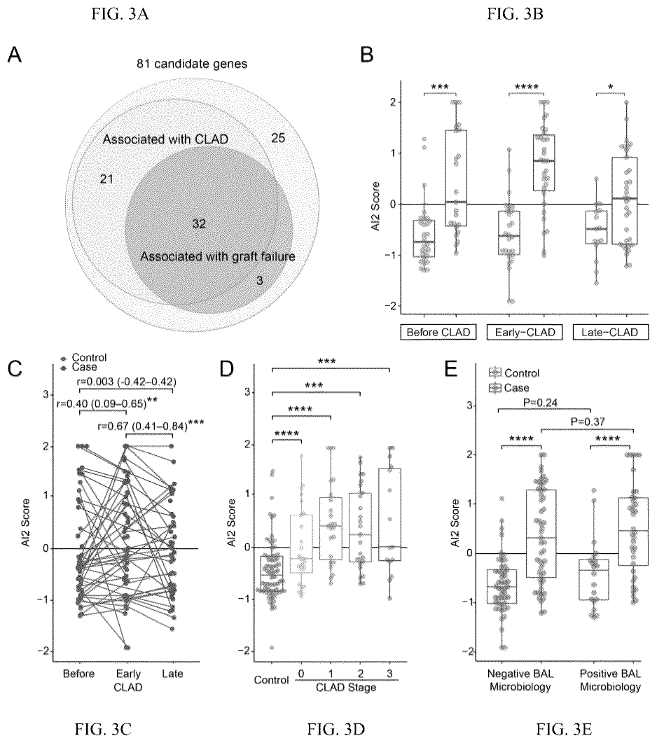

While all four candidate metagenes showed promise for the identification of CLAD, given their diverse origin, some of the component genes might be associated with non-CLAD processes. Thus, genes not associated with CLAD or graft outcomes were eliminated. Of the 81 genes in the union of these four transcript sets: 53 genes were positively associated with CLAD, 35 genes were associated with graft failure risk, and 32 genes showed linkage to both CLAD and graft failure, as depicted in the Venn diagram in FIG. 3A. All these genes were associated with CLAD status after adjusting for time post-transplant, except for midnolin. Together, there was increased expression of the AI2 metagene in all three time points (FIG. 3B). To see how stable the AI2 was over time, AI2 scores were plotted with lines indicating individual subjects (FIG. 3C). Within cases and controls in the derivation cohort, a low positive correlation was observed before CLAD to early CLAD and a moderate positive correlation between early and late CLAD. There was no association from the before to late time groups.

To determine how airway infection might influence this AI2 score, an analysis was performed stratified by infection, assessed by the identification of a potential pathogen on clinical microbiology testing. As shown in FIG. 3E, the AI2 score distinguished CLAD cases from controls in both infection and non-infection brushes, while there was no statistically significant increase in AI2 score associated with clinical detection of BAL pathogens a priori considered pathogenic.

Although the derivation cohort was designed to explore changes with respect to time of CLAD onset, CLAD severity is typically staged based on the degree of FEV1 loss from post-transplant baseline. Whether the AI2 score varied with CLAD stage was explored. As shown in FIG. 3D, there was an increase in AI2 score for participants categorized as CLAD cases at all CLAD stages (including stage 0, representing those who would soon go on to develop CLAD), as compared with the control group. However, the median AI2 score was highest at CLAD stage 1, which may indicate that inflammation was greatest in those with a low degree of functional decline at the time of brush.

Finally, how AI2 scores compared between restrictive (RAS) and obstructive (BOS) subtypes of CLAD was explored, as airway brushes sample bronchioles that would be most impacted by BOS, and whether past acute cellular and lymphocytic bronchiolitis rejection impacted AI2 score. AI2 scores were increased in all three CLAD subtypes, but the differences between cases and controls were less for the RAS subtype (FIG. 3E). AI2 scores were not significantly increased in individuals with a history of past rejection (effect estimate 0.69, P=0.18).

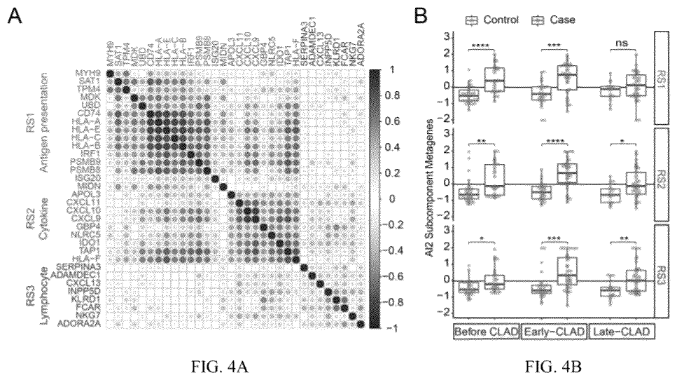

Hierarchical clustering of these 32 AI2 genes resulted in three co-expression clusters, which were labeled RS1, RS2, and RS3 (FIG. 4A). Gene Ontology pathway analysis found that RS1 enriches for antigen presentation pathways, RS2 for cytokine responses, and RS3 for lymphocyte activation (p<0.001 for each). Statistically significant differences in AI2 subcomponent metagene scores between CLAD and non-CLAD subjects were found for RS1 at the before CLAD timepoint, for RS2 at the early CLAD timepoint, and for RS3 at the late CLAD timepoint. (FIG. 4D).

Comparison with an Unbiased CLAD Classifier Across Timepoints

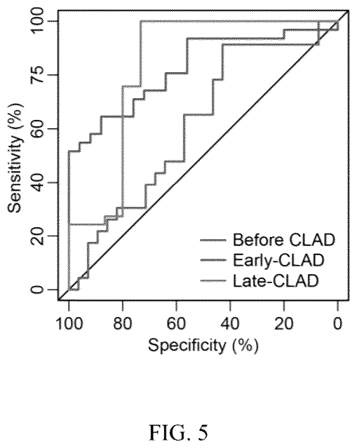

Using leave-one-out cross validation with a gradient boosted tree model, the late-CLAD time point now had this highest AUC (0.83 versus 0.82 in early CLAD and 0.61 in the before CLAD group. FIG. 5). Comparing these curves, the early CLAD and late unbiased classifiers had a greater AUC than the pre-CLAD classifier (P=0.03 and P=0.06, respectively), suggesting that strong performance of the AI2 score before CLAD could be a result of the candidate gene approach.

### Validation of the AI2 Score as a CLAD Classifier

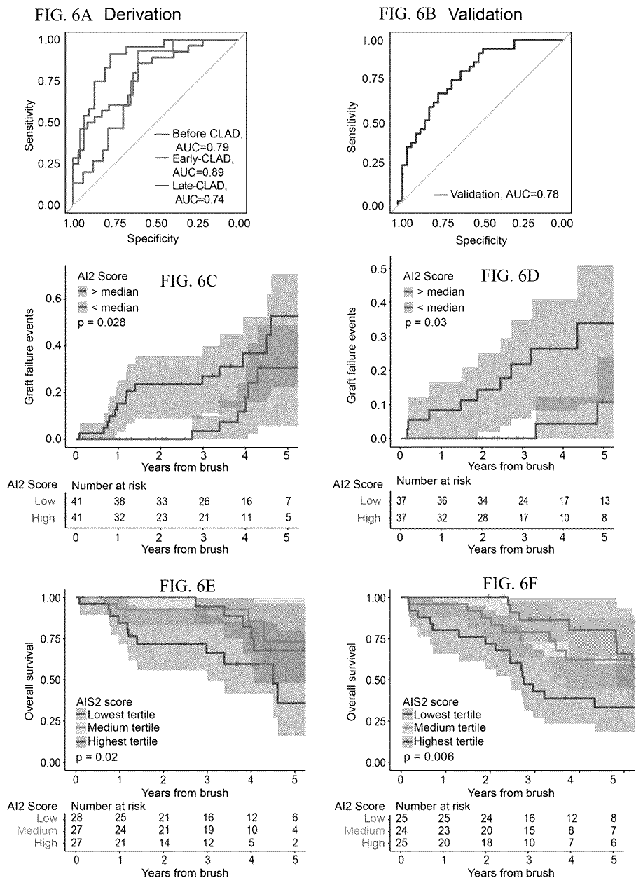

To assess the potential of the AI2 score as a CLAD biomarker, AI2 score was examined in the derivation cohort and in an independent validation cohort from a separate institution. Subject characteristics for the external validation cohort are shown in Table 3. ROC analyses was used to gauge the potential sensitivity and specificity of the AI2 score in distinguishing CLAD cases and controls. Because the cohorts were designed with a balance of cases and controls, a pre-specified threshold of 0 was used to assess sensitivity (SE), specificity (SP), positive predictive value (PPV), and negative predictive value (NPV). The AI2 score yielded an AUC in the before-CLAD cohort of 0.79 (95% CI 0.66-0.91, SE: 0.89, SP: 0.52, PPV: 0.69, NPV: 0.80). In the early CLAD cohort, the AUC for discriminating CLAD cases and controls was 0.89 (95% CI 0.80-0.97, SE: 0.81, SP: 0.88, PPV: 0.79, NPV: 0.89) and in the late-CLAD cohort, the AUC was 0.74 (95% CI 0.60-0.88, SE: 0.57, SP: 0.84, PPV: 0.66, NPV: 0.78) (FIG. 6A). In an external validation cohort of 37 CLAD cases and 37 controls from another institution, the calculated AI2 score yielded an AUC of 0.78 (95% CI 0.68-0.88) (FIG. 6B). 3.5 AI2 score association with time to graft failure and retransplant-free survival.

From the latest available brush in the derivation cohort, a one standard deviation increase in AI2 score increased the hazard ratio for graft failure by 1.97-fold (95% CI 1.39-2.78-fold, 20 events, P=0.0001, FIG. 6C). After adjusting for baseline subject characteristics, DSA, ACR, and BAL culture positivity in a multivariable model, this hazard ratio was 1.93-fold (95% CI 1.29-2.88, P=0.001). In the validation cohort, each 1-point increase in AI2 score was associated with a 2.44 (95% CI 1.44-4.13)-fold increase in the risk of graft failure (12 events, P=0.0009, FIG. 6D). There was no association between AI2 score and death from other causes in the derivation cohort (11 events, P=0.49), but there was an unexpected increased risk of non-CLAD death in the validation cohort (HR 1.44, 95% CI 1.01-2.05, 21 events). Limited to CLAD cases, a pooled analysis of the derivation and validation cohorts found a 1.65-fold (95% CI 1.14-2.38, P=0.008, 28 events) increased risk of graft failure per standard deviation increase in AI2 score.

In the derivation cohort, each 1-point increase in AI2 score was associated with a 1.43-fold (95% CI 0.99-1.89) increase in the risk of death that approached statistical significance (P=0.06, 31 events, FIG. 6E). After adjustment for participant characteristics, ACR, DSA, and BAL culture positivity, the hazard ratio for death or retransplantation was 1.56 (95% CI 1.05-2.33, P=0.03). In the validation cohort, each 1-point increase in AI2 score was associated with a 1.77-fold increase in the risk of death or retransplantation (95% CI 1.31-2.40, 33 events, P=0.0002) (33 events, P=0.0002, FIG. 6F). After adjusting for participant characteristics, the hazard ratio was 2.47 (95% CI 1.61-3.78, P<0.0001). Finally, in a pooled analysis including only CALD cases, increasing the AI2 score was associated with a 1.44-fold increased risk of death or retransplantation (HR 1.44, 95% CI 1.09-1.91, P=0.01, 47 events).

More stringent gene selection criteria resulted in smaller gene lists but similar associations with outcomes in the validation cohort. Specifying an exchangeable covariance structure resulted in a score of 17 genes (CXCL9, CXCL10, CXCL13, GBP4, HLA-A, HLA-B, HLA-C, HLA-E, IDO1, KLRD1, MDK, NLRC5, PSMB8, PSMB9, TAP1, TPM4, UBD), which had an unadjusted death or retransplantation hazard ratio of 1.69, P=0.0008. Increasing the significance threshold resulted in a partially overlapping list of 17 genes: ADAMDEC1, CXCL11, CXCL13, GBP4, HLA-A, HLA-E, HLA-F, IDO1, IRF1, KLRD1, MDK, NKG7, NLRC5, PSMB9, SAT1, TAP1, and TPM4; and resulted in a HR of 1.74, P=0.0004.

### DISCUSSION

Airway transcriptome inflammatory gene signatures identified CLAD, even when the brush was done before the time of CLAD onset. A hybrid AI2 score comprised of genes associated with antigen presentation, cytokine signaling, and lymphocytic inflammation was identified. Increased AI2 scores were associated with CLAD in airway brushes taken before CLAD, early and late after CLAD onset, and in an independent validation cohort. Further, AI2 scores predicted time to graft failure and death in derivation and validation cohorts. Given the limitations of histopathological analysis of transbronchial biopsies for diagnosing CLAD, these data suggest that transcriptional analysis of airway brushing could provide diagnostic and prognostic data in lung transplant recipients (8). Recipients with high AI2 scores may be particularly suitable candidates for clinical trials to prevent CLAD development or progression.

This study's longitudinal design can allow the analysis to determine how biological processes of airway inflammation change in individuals and over the course of CLAD (14). In the before CLAD time point, prominent increases were observed in genes associated with antigen presentation, followed by those associated with cytokine signaling in the early CLAD time point, and lymphocyte activation in late CLAD. Of the candidate gene scores, the lymphocytic bronchitis score performed best in the before CLAD cohort and the Pitt score worked better in the cohort early after CLAD onset, which would be consistent with the original cohort designs from which these gene signatures were derived (9, 12). The AI2 score performed least well in the late CLAD cohort and, when compared across CLAD FEV1 severity stages, the AI2 score peaked at CLAD stage 1.

Together these findings show advanced CLAD may have less prominent inflammatory features. An alternate interpretation of these findings is that CLAD onset is associated with acute rejection that can be detected by AI2 score but is not reliably seen by histopathology. Unbiased machine learning approaches demonstrated that advanced CLAD did have gene expression changes that could be used to separate cases from time-matched controls. Such features likely involve epithelial cell-specific changes, such as the response to wounding pattern observed in CLAD transbronchial biopsies (19), that would not be captured by a rejection/inflammation candidate gene approach.

While several genes can be shown to be differentially expressed between groups of lung transplant recipients with and without CLAD, substantial heterogeneity was observed in CLAD molecular patterns between individuals (see FIG. 4A). The metagene score approach, evaluating the sum of normalized gene expression for several genes attempts to address that heterogeneity. The AI2 subcomponents identified here based on patterns of co-expression may reflect underlying molecular endotypes of CLAD, which may or may not reflect heterogeneous CLAD clinical phenotypes (20).

The AI2 score identified recipients at risk for future graft failure and death across two cohorts, despite differences in management protocols and transplant candidate populations. Recipients at Pitt may have more profound telomere dysfunction amongst those with IPF (21, 22). In non-IPF recipients, Pitt uses alemtuzumab for induction, whereas UCSF uses basiliximab for all recipients (23).

While such recipients may be undergoing workup for possible infection, there were no increased rates of infections associated with the AI2 score. Larger studies may identify associations between select pathogens or stricter definitions of infection and molecular airway inflammation. There is potential for survival bias as recipients with progressive CLAD may not survive to be eligible for a late CLAD brush. Finally, the late CLAD group may be exposed to salvage immune suppression, including mTOR inhibitors that might impact gene expression (24).

### B. Example 2: Lung Transplant Antibody Mediated Rejection is Diagnosed by AI2 Score

Antibody-mediated rejection (AMR) in lung transplant is diagnosed based on hallmark features of graft dysfunction, complement deposition, donor specific antibodies, and consistent histopathology. AMR can drive rapid graft failure and mortality, but often is more indolent, and challenging to distinguish from other forms of graft dysfunction. Airway brush gene expression identifies patterns associated with chronic lung allograft dysfunction (CLAD). Small airway brush transcriptomes for 11 participants with active AMR and 22 references with stable lung function and no AMR features were assessed. Differential expression analysis was performed on small airway brushings and scores were computed for AI2 and other relevant rejection-associated transcript sets. Associations with AMR features by multivariable models and time to graft failure by Cox proportional hazards modeling were examined. AMR histology, DSA, C4D, and graft dysfunction were associated with increased AI2 score (P<0.05, for each). C4D binding and FEV1 decline were the only AMR features associated with graft failure. However, the AI2 score predicted time to graft failure (p=0.0024).

Thus, AMR is associated with increases in AI2 score in small airway brushes from lung transplant recipients, an expression pattern overlapping with CLAD. Airway inflammation gene expression can predict outcomes in transplant recipients with concern for AMR.

In summary, the AI2 metagene in small airway brushings identifies CLAD before its spirometric diagnosis and predicts subsequent graft outcomes, with less risk than has been reported from transbronchial biopsies.

### C. Example 3

Acute lung allograft dysfunction (ALAD) is a clinical syndrome of FEV1 decline concerning for chronic lung allograft dysfunction (CLAD) onset. Novel diagnostic tools are needed to identify those with ALAD who will progress to CLAD and to target appropriate therapies.

12 recipients with ALAD and 8 controls with stable allograft function were prospectively identified for small airway brushing and single cell RNA sequencing analysis using 10×. ALAD cases were stratified as recovered (28), persistent (29), or progressive (27) FEV1 decline. Cell compositional changes, pseudobulk Reactome pathways, and the AI2 score, previously linked to CLAD in airway brush transcriptomes, were assessed as a function of ALAD outcome.

### Study Design

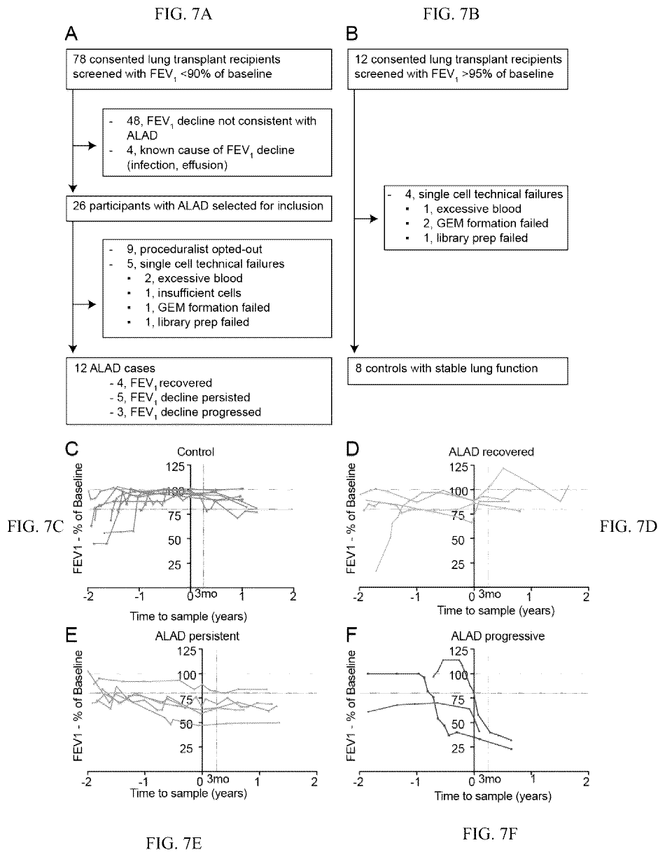

Lung transplant recipients at least one year post transplant, and who were scheduled for bronchoscopy were screened based on FEV1 as a percent of their post-transplant baseline (mean of two highest measurements of FEV1 post-transplantation >21 days apart) (31) using an automated algorithm (FIG. 7A). Recipients with a recent >10% decline in FEV1 who had clinical documentation of suspected ALAD or new onset CLAD were selected for prospective airway brush collection for single cell RNA sequencing. Recipients with a clear alternate cause of FEV1 decline, such as pneumonia or effusion, or low clinical suspicion for CLAD were excluded. In some unstable recipients, airway brushes were deferred to prioritize clinically necessary tissue samples. A control cohort with FEV1 >95% of baseline without chart documentation concerning for graft instability was recruited in parallel (FIG. 7B). Participants were followed for at least 12 months and outcomes from ALAD were retrospectively stratified as: “ALAD recovered” participants, who returned to near-baseline FEV1 following the ALAD episode; “ALAD persisted” participants, who had a loss of FEV1 that stabilized following bronchoscopy; and “ALAD progressive” participants who continued to have further FEV1 decline (>10% decline in FEV1 within one year of transplant) leading to graft failure following the ALAD episode.

### Airway Brush Single Cell RNA Sequencing

During bronchoscopy, two 2-mm cytology brushes (Conmed #129) were inserted and retracted 10 times at 1 to 2 cm from the periphery then cut into a tube of culture media. Cell were dissociated, strained and resuspended for loading onto a 10× chip. Gel Bead-In Emulsions (GEMs) formed in a 10× Chromium controller were immediately collected for cDNA amplification. Completed libraries were sequenced in two separate batches on the NovaSeq 6000, with a targeted depth of 50,000 reads/cell.

### Single Cell RNA Seq Data Analysis

Completed libraries were aligned using Cell Ranger v7.0.1, with GRCh38 (2020A) as reference genome. Multiplexed samples were demultiplexed using SoupOrCell and individual samples were identified via SNP matching from genotyping data. Ambient RNA and doublets were removed by SoupX (v1.6.2) and DoubletFinder (v2.0.3), respectively.

Seurat (v4.3.0) was used for downstream analysis. Cells were clustered with the Louvain algorithm and identified using previously reported canonical markers (32). ALAD outcome group was treated as a continuous variable, coded as 1: control, 2: recovered, 3: persisted, and 4: progressive. Differential expression analysis of ALAD outcome grouping as a function of gene count was performed using edgeR for each cell cluster, using a minimum library size of 5,000, and minimum total gene count of 20. Gene set enrichment analyses were performed using the Reactome pathway database (33).

### Statistical Analyses

The type of statistical test is specified in the figure legends. As the cohorts were small, conservative non-parametric tests were used. For PERMANOVA analysis, cell count distances were calculated using the robust Aitchison method and tested as a function of ALAD outcome group as a continuous variable. Spearman correlation coefficients were used to assess associations between multidimensional scaling (MDS) components and cell frequencies and between cell frequencies and ALAD outcome grouping.

For contrasts with value per sample, Spearman correlations were performed with ALAD outcome group coding as the independent variable (continuous predictor), and the variable of interest as the independent (for example, the frequency of cell subsets), with no correction for multiple comparisons.

AI2 gene set was calculated per cell using Seurat's module score method. When contrasting AI2 score per cell across cohorts, linear mixed effects regression (LMER) was performed using the lme4 package (v3.1-3), which can account for intra-correlations between repeated measurements (cells) within a subject. Sample of origin was accounted for by inclusion of patient identification as random effect. The ALAD outcome grouping (continuous predictor) was included as the fixed effect.

### Results

Prospective Enrollment of Individuals with ALAD for Single Cell RNA Sequencing

Chart review was triggered for 78 recipient bronchoscopies based on an FEV1 value below 10% of post-transplant baseline lung function (FIG. 7A). Recipients were excluded based on the absence of documented clinician concern for new onset CLAD or ALAD (n=48) or the presence of known causes of lung dysfunction (n=4). A total of 26 individuals with ALAD were prospectively selected for single cell RNA sequencing on collected airway brushes. Investigational brushes were not available due to proceduralist preference (n=9) or technical failures (n=5). Of 12 stable controls (FEV1 >95% of baseline) for whom brushes were collected, 4 of the samples were not processed (FIG. 7B). The resulting cohort included 12 participants with ALAD, and 8 control participants with stable lung function.

Participant characteristics are shown in Table 5. Donor specific antibodies, all of which were class II, were seen in some participants in the persistent and progressive cohorts, although not statistically significant. All transbronchial biopsies were ISHLT grade A0B0.

TABLE 5
Participant Characteristics

| | | | ALAD | | ALAD | | ALAD | | P- |
|---|---|---|---|---|---|---|---|---|---|
| | Control | | recovered | | persistent | | progressive | | value |
| n | 8 | | 4 | | 5 | | 3 | | |
| Age, median [IQR] | 64.5 | [61, 65] | 60.5 | [60, 61] | 61 | [61, 67] | 49 | [46, 56] | 0.31 |
| Donor Age, median [IQR] | 40 | [35, 56] | 35 | [28, 44] | 49 | [31, 59] | 30 | [30, 34] | 0.54 |
| Male, N (%) | 3 | (37.5) | 2 | (50) | 4 | (80) | 2 | (66.7) | 0.48 |
| Male Donor, N (%) | 5 | (62.5) | 3 | (75) | 5 | (100) | 3 | (100) | 0.31 |
| Recipient ethnicity, N (%) | | | | | | | | | 0.43 |
| White | 5 | (62.5) | 2 | (50) | 4 | (80) | 1 | (33.3) | |
| Black | 1 | (12.5) | 0 | (0) | 0 | (0) | 0 | (0) | |
| Hispanic | 2 | (25) | 1 | (25) | 0 | (0) | 0 | (0) | |
| Asian | 0 | (0) | 0 | (0) | 0 | (0) | 1 | (33.3) | |
| Multiracial | 0 | (0) | 1 | (20) | 1 | (20) | 1 | (33.3) | |
| Transplant indication, N (%) | | | | | | | | | 0.49 |
| A, Obstructive | 2 | (25) | 2 | (50) | 1 | (20) | 0 | (0) | |
| D, Restrictive | 6 | (75) | 2 | (50) | 4 | (80) | 3 | (100) | |
| Donor/Recipient cytomegalovirus serostatus, N (%) | | | | | | | | | 0.15 |
| D−/R− | 2 | (25) | 1 | (25) | 4 | (80) | 0 | (0) | |
| R+ | 3 | (37.5) | 3 | (75) | 1 | (20) | 2 | (66.7) | |
| D+/R− | 3 | (37.5) | 0 | (0) | 0 | (0) | 1 | (33.3) | |
| Single lung, N (%) | 0 | (0) | 1 | (25) | 0 | (0) | 0 | (0) | 0.24 |
| Brush months from | 18.6 | [16.7, 23.3] | 38.0 | [31.5, 56.3] | 46.7 | [34.2, 51.6] | 28.2 | [18.9, 34.2] | 0.02 |
| transplant, median | | | | | | | | | |
| [IQR] | | | | | | | | | |
| Follow up months | 12.1 | [11.7, 13.6] | 12.2 | [11.3, 14.4] | 14.6 | [13.5, 15.6] | 11.1 | [7.3, 11.1] | 0.16 |
| from brush, median | | | | | | | | | |
| [IQR] | | | | | | | | | |
| Donor specific | 0 | (0) | 0 | (0) | 2 | (40) | 1 | (33.3) | 0.15 |
| antibodies, N (%) | | | | | | | | | |

FEV1 trajectories for stable controls showed stable FEV1 except for one participant who contracted SARS-CoV-2 1 month following the bronchoscopy and had associated decline of FEV1 (FIG. 7C). Of 12 patients with ALAD, the FEV1 recovered to baseline following the ALAD episode in four (33%), classified as “ALAD recovered” (FIG. 7D). Five (42%) participants had stabilization of FEV1 at a lower baseline, classified as “ALAD persisted” (FIG. 7E). In three (25%) of participants with ALAD, the FEV1 continued to drop after the bronchoscopy—this group was termed “ALAD progressive”. The three participants in this group proceeded to repeat transplant (n=1) or death (n=2) within one year of sample collection (Table 5). All but one participant in the ALAD persistent group, and all ALAD progressive participants met the criteria for CLAD (31) three months following sample collection.

ALAD Outcome is Associated with Changes in Cell Composition

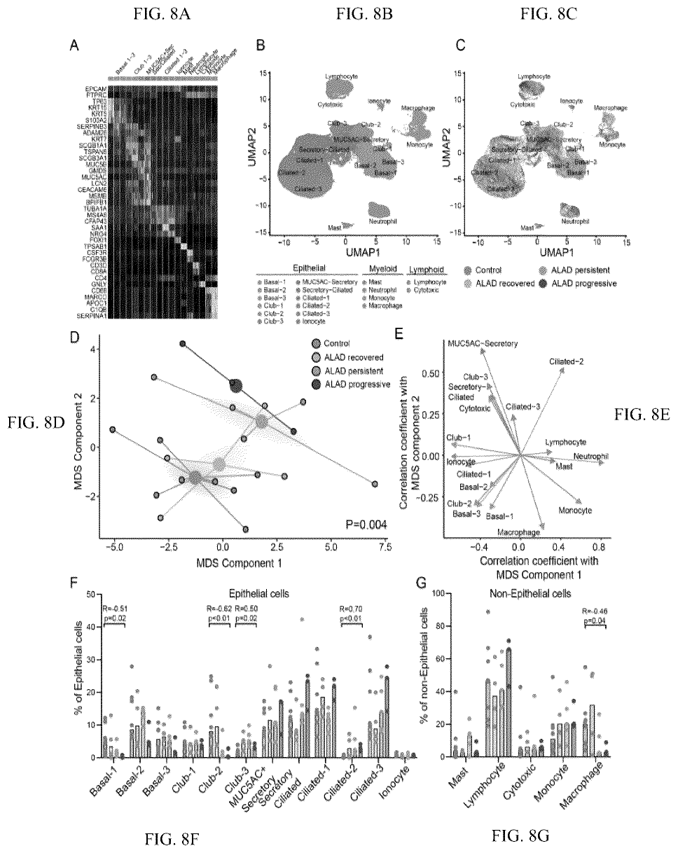

Of the 68,140 analyzable cells from 20 recipients, around 80% were epithelial cells (EPCAM+), which encompassed 12 distinct clusters. Using canonical markers, three basal cells clusters, three club cell clusters, one secretory cluster with MUC5AC expression, one cluster transitioning from secretory to ciliated, three ciliated clusters, and a small population of ionocytes (FIG. 8AB) were identified. The remaining 20% of cells were leukocytes (PTPRC+), and included two lymphocyte clusters, and single clusters of macrophages, monocytes, mast cell, and neutrophils (FIG. 8AB). Neutrophil counts had high variability, which could reflect biological variation (34) or poor viability during processing (35), and this cluster was excluded from comparisons of cell proportions.

UMAP plots suggested cell compositional differences linked to worse ALAD outcome groupings, such as a loss of the rightmost basal and club cell populations (FIG. 8C). To examine the global impact of ALAD outcome grouping on cell beta-diversity, MDS analysis of cell cluster frequencies was performed. Distributions of small airway cell populations differed significantly across ALAD groups (PERMANOVA P=0.004, FIG. 8D). Cells from control and ALAD recovered groups largely overlapped, while ALAD progressive recipients had greater deviations from controls. To understand the drivers of these cell composition differences, the correlation coefficients between cell frequency and MDS components were plotted (FIG. 8E). The control group showed a greater association with club clusters and macrophages, while non-resolving ALAD groups were associated with ciliated populations.

The proportions of cell populations across ALAD outcome groups was compared. There were no observed differences in the proportion of immune cells relative to the total sequenced population. Within the epithelial cells, worst ALAD outcome grouping had decreases in the proportion of basal-1 and club-2 subsets and increases in club-3 and ciliated-2 populations (FIG. 8F). No differences in epithelial subsets proportions were observed in the control participants compared to the ALAD recovered group.

Among the non-epithelial cells, the frequency of macrophages decreased among patients with non-recovering ALAD (FIG. 8G). The proportion of all other non-epithelial subsets were similar between controls and ALAD recovered.

Taken together, these data reveal shifts in small airway cell composition during the ALAD events in recipients with persistent or progressive loss of lung function, but not in participants with recovered ALAD function.

Epithelial cell subtypes are primary drivers of CLAD-associated gene expression.

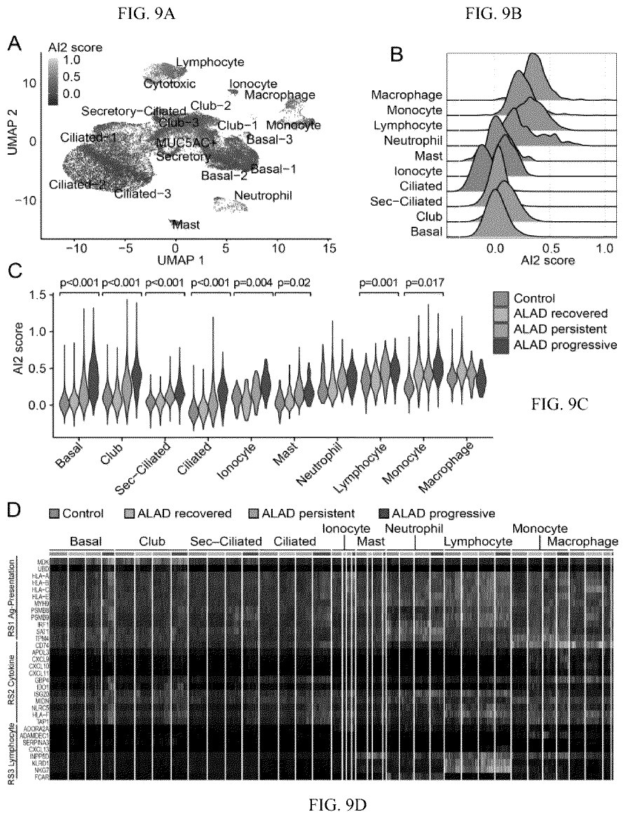

A set of 32 genes associated with CLAD and graft failure, termed the AI2 score (30) was identified. Across all cell types in ALAD control recipients, the AI2 score was most expressed in immune cell populations, as expected given the immune cell-associations of some of the AI2 genes (FIG. 9A). Among epithelial cells, it was observed that club cells tended to have higher AI2 scores, while ciliated cells had the lowest (FIG. 9B).

AI2 expression change with ALAD outcome within cell subsets was next examined (FIG. 9C). In every epithelial subset, the AI2 score increased with worsening ALAD outcome, where progenitor-type cells (basal and club) tended to have the greatest increase. Among the immune cell populations, increases in AI2 were observed in lymphocytes and monocytes only, with increases more modest than in epithelial cells. ALAD recovered AI2 profiles was similar to controls for all cell types.

Individual AI2 genes were assessed across cell types and ALAD status (FIG. 9D). Genes related to antigen presentation, interferon response, and IDO1 were primarily increased in epithelial cell subsets. Lymphocyte subsets primarily expressed INPP5D, KLRD1 and NKG7, while CD74 was primarily from monocytes.

Taken together, these results confirm an increase in AI2 score in individuals with unresolving ALAD, with the most statistically significant increases coming from epithelial cell subsets.

ALAD outcome is associated with immune activation and impaired homeostasis.

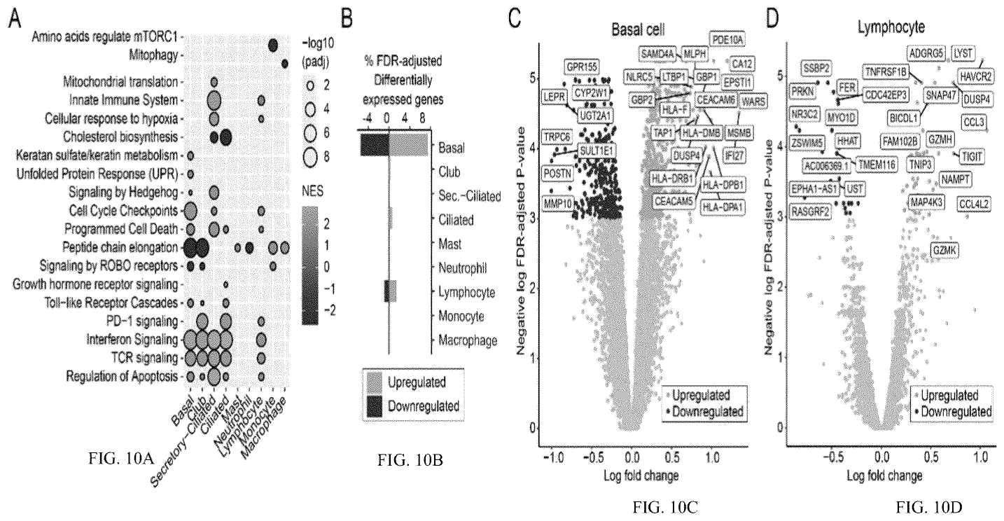

Gene set enrichment analysis (GSEA) of Reactome pathways associated with ALAD outcome was performed for each cell subset. Interferon and TCR signaling/antigen presentation pathways were strongly associated with ALAD progression in epithelial cells and in lymphocytes (FIG. 10A). In addition, pathways associated with apoptosis and halting of cell proliferation were increased in epithelial populations. Basal cells had diminished peptide synthesis and ciliated cells had decreased cholesterol biosynthesis, which may be consistent with impaired homeostasis. The secretory-ciliated group also had the greatest upregulation of innate immune responses, and responses to hypoxia.

To identify cells with the most differentially expressed genes (DEG), the percentage of all genes detected in the subset that were differentially expressed (FDR <0.1) was calculated as a function of ALAD outcome grouping (FIG. 10B). The subset with the greatest percentage of DEG were basal cells (FIG. 10C), with an increase in those associated with secretory cells (CEACAM6, CEACAM5), suggesting dedifferentiation, and with antigen presentation (TAP1, HLA molecules). Lymphocytes had the second largest fraction of DEG, with increases in inhibitory molecules (TIGIT, HAVCR2), consistent with PD-1 signaling, as well as activation and cytotoxicity genes (GZMH, CCL3) (FIG. 10D). Few DEG were detected in club, secretory-ciliated, and ciliated cells. Secretory-ciliated cells had increased MHC I genes (HLA-A B C), non-classical MHC genes (HLA-E/F), and interferon-associated genes (GBP1, WARS). Ciliated cells upregulated genes related to antigen presentation (TAP1, PARP9, NLRC5).

### Immune Compartment Compositional Changes

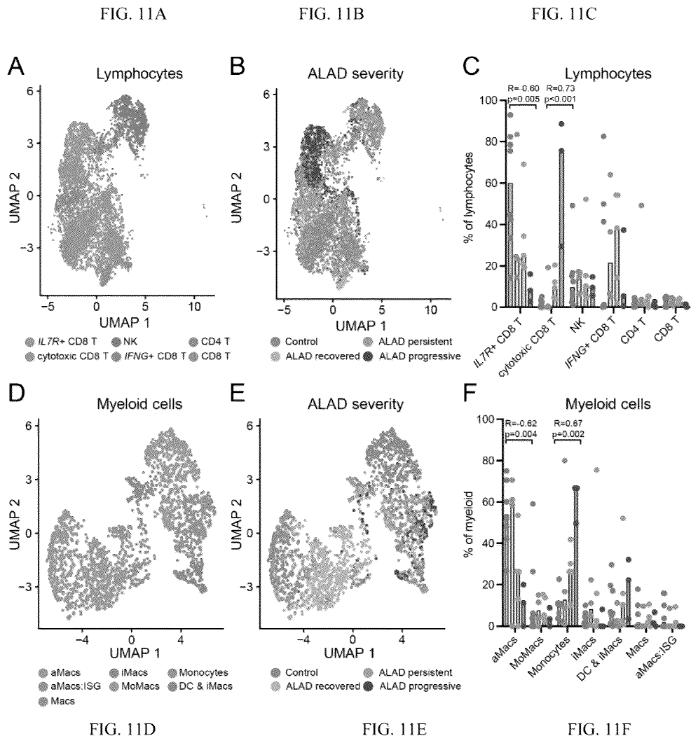

Given evidence of immune-activating signals from the epithelial cells, the immune cells were characterized further. Reclustering of both lymphocyte populations yielded 6 clusters (FIG. 11A). Most clusters were subtypes of CD8+T (cytotoxic, IL7R+, or IFNG+), although it was observed a CD4+T and a NK cell cluster. Cell composition differed based on ALAD outcome (FIG. 111B). While the IL7R+ population was the most prevalent CD8 T cell population in the control participants, it was largely lost in progressors, replaced by cytotoxic CD8+ T cells, with a trend for IFNG+CD8+ T cells as well (FIG. 11C).

Myeloid cells were reclustered, revealing 7 types of myeloid cells. There were two resident macrophage types: alveolar macrophages (aMacs)—one cluster having high expression of ISG, as recently described (36) and interstitial macrophages (iMacs) with low MHC-II expression. Other myeloid clusters included monocyte-derived macrophages (MoMacs), monocytes, an unidentified cluster of macrophages, and a cluster with high HLA expression, which included both dendritic cells (DC) and iMacs (FIG. 11D). Worst ALAD outcome demonstrated a sharp decrease in aMacs, and an increase in monocytes (FIG. 11EF). Of note, the aMacs expressed anti-inflammatory markers (37).

### DISCUSSION

Persistent and progressive ALAD were associated with substantial changes in cell composition and cell-specific gene expression in the small airways. Poor outcomes from ALAD were associated with a loss of well-differentiated club and basal cell subsets. The remaining basal cells had upregulation of antigen presentation and differentiation towards a more secretory phenotype that could be indicative of a reparative response to injury. The AI2 score (30), developed as an airway brush transcriptome predictor of graft-failure risk, was most prominently upregulated in basal and club cells in association with progressive ALAD. Worse ALAD outcomes were also associated with a loss of quiescent CD8+ T cells and increases in activated CD8+ T populations, while myeloid populations were depleted of alveolar macrophages in favor of monocyte-derived macrophages. These findings indicate the important biological features of non-resolving ALAD.

The concept of ALAD was proposed in 2013 as a syndrome of 10% decline in FEV1 concerning for incipient CLAD (27). However, as a 10% decline in FEV1 may reflect unusual variation in spirometry, ALAD was defined based on both FEV1 decline and documented clinical concern, with a goal of enhancing specificity. No significant airway cell compositional or transcriptional changes between ALAD recovered and control recipients was observed, suggesting an absence or transience of molecular changes of with resolving ALAD. By contrast, FEV1 decline paired with airway inflammation was associated with poor outcomes, consistent with our prior observations (29, 30). These data evidence that biomarkers of allograft injury can risk-stratify declines in lung function (14, 15). At the same time, larger studies may identify ALAD endotypes with distinct molecular pathogenesis and natural histories (29, 30, 40).

While other lung transplant tissues have been examined by single cell sequencing (36, 37, 41, 42), small airway brushes during ALAD may uniquely capture the epicenter of CLAD (29, 43, 44). CLAD explant tissue showed similar features including upregulation of interferon responses, antigen presentation, and apoptosis in basal cells, increases in cytotoxic T cells, and a loss of macrophages (41). Enrichment of cytotoxic CD8+ T cells has also been observed in flow cytometric analysis of small airway cells from CLAD lungs (40) and in the BAL of participants with ALAD (39). The loss of a potentially immune-suppressive BAL macrophage population associated with ALAD progression was replicated here in airway brushes (37). However, no significant differences in frequency of the ISG+ alveolar macrophages, also recently linked to ALAD (36), was observed.

The genes in the AI2 score were analyzed based on their prior associations with graft failure (30). Single cell approaches may help address heterogeneity attributable to cell compositional differences, and some of the heterogeneity in the AI2 score subcomponents does appear to map to cell compositional variation. In contrast to our expectation that many of these genes be driven by immune cell influx, the epithelial cell compartment was the major source AI2 gene expression changes. CXCL9/10 molecules, for example, are linked to interferon responses primarily in myeloid cells (45), but these chemokines can be produced by epithelial cells, as observed here. IDO1, a key tolerance mediator in lung transplant rejection (40, 46), was primarily upregulated in basal and club cells with severe ALAD. As expected, NKG7 and KLRD1 were primarily seen in lymphocytes and may reflect useful markers or therapeutic targets for higher risk lymphocytic inflammation (47). CD74 can be upregulated on epithelial cells, but here it was primarily expressed by monocytes (48).

In summary, this 68,140-cell dataset identified cell compositional and transcriptional changes associated with persistent or progressive ALAD. The findings suggest that interventions targeting interferon-dependent antigen presentation, macrophage/T cell dysregulation, and associated basal/club cell dedifferentiation may hold promise as interventions during episodes of lung function decline in lung transplant recipients.

### D. Example 4

Most cells from airway brushes are epithelial subsets, accompanied by 30-40% monocytes, and ˜10% lymphocytes. To understand the link between differential gene expression and cell types, genes in bulk RNA sequencing were labeled according the cell with the highest relative expression of that gene in the lung cell atlas (50). From this, AI2 score was observed to be positively correlated with myeloid- and lymphoid-associated genes and negatively correlated with epithelial-associated genes (ANOVA <0.01). Highly expressed genes that were negatively correlated with the AI2 score and reflected several epithelial subtypes were selected: SCGB3A1 (secretory/club), MS4A8 (ciliated), KRT5 (basal), CALM1 (transitional epithelial).

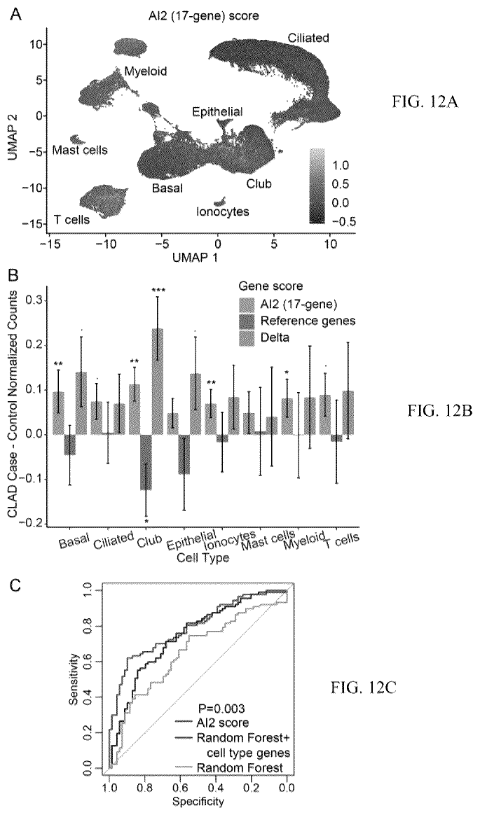

Data from single cell RNA sequencing from prospectively identified airway brush recipients was examined. To enrich for those at risk of developing CLAD, brushes from individuals with Acute Lung Allograft Dysfunction (ALAD), with ALAD defined as a 10% decline FEV1 over the past 6 months were collected. ALAD diagnosis was confirmed based on chart review of clinician documentation of ALAD as an indication for bronchoscopy. 5 cases of ALAD who progressed to CLAD, 7 who recovered normal lung function, and 8 stable controls, were identified at UCSF as part of a CFF-funded research study. As shown in FIG. 12A, the AI2 score was expressed in multiple airway brush subsets, including club cells, T cells, myeloid cells and subsets of basal and ciliated cells. Module score analysis was performed within each cell type for the AI2 gene set, and their difference, using mixed effects models. As shown in FIG. 12B, the AI2 score was highest in recipients with ALAD who declined, with high statistical significance in Basal, Club, Ciliated and Transitional epithelial cell subsets. The reference score was found to be negatively associated with CLAD, particularly in Club cells, consistent with an extensive literature linking club cell dysfunction and CLAD (51). ALAD outcomes were best predicted when the reference gene set was subtracted from the AI2 gene set. Thus, this reference gene set can be used to normalize AI2 gene scores.

The identification of appropriate reference genes reflecting the cells present in most airway brushes, particularly in states of health (SCGB3A1, MS4A8, KRT5, and CALM1) permits an important advance on the AI2 classifier paradigm. Normally, RNAseq genes are normalized based on the overall number of counts, making it difficult to translate RNAseq findings to lower-cost, rapid turnaround assays, such as quantitative PCR, reverse transcription-loop mediated isothermal amplification (LAMP), or digital RNA counting methods. However, with an appropriate set of reference genes, assays based on unnormalized counts that would derive from more clinically applicable sequencing technologies can be developed.

The power of these systems can be further increased by using artificial intelligence machine learning algorithms. Random forest machine learning algorithm was used to develop a classifier of CLAD cases versus controls based on the log of (counts+1) for genes in the AI2 gene set. Reference genes for secretory (SCGB3A1), ciliated (MS4A8, CALM1), basal (KRT5, TP63), leukocyte (PTPRC, MARCO, GNLY) were added. To ensure that these random forest models were not trained on the data from the same subject as the test data, a modified leave-one-out cross validation strategy was used. These models used 500 trees with replacement. To assess accuracy of CLAD classifiers, area under the receiver operating curve (AUC) was calculated, with the DeLong method for assessing statistically significance and error. For comparison, the AI2 score had an AUC of 0.79 (95% CI 0.72-0.86), while a random forest model trained on unnormalized counts had an AUC of only 0.65 (95% CI 0.57-0.74). However, adding the above cell type reference genes boosted the AUC to 0.74 (95% CI 0.67-0.83, P=0.0003 versus unnormalized, FIG. 7C). Relative importance diagnostics on these random forest classifiers identified SCGB3A1 and PTPRC, and MARCO as top genes, indicating the importance of quantifying epithelial, immune, and macrophage subsets.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Verleden G M, Glanville A R, Lease E D, Fisher A J, Calabrese F, Corris P A, Ensor C R, Gottlieb J, Hachem R R, Lama V, Martinu T, Neil D A H, Singer L G, Snell G, Vos R. Chronic lung allograft dysfunction: Definition, diagnostic criteria, and approaches to treatment-A consensus report from the Pulmonary Council of the ISHLT. J Heart Lung Transplant 2019; 38: 493-503.
2. Kulkari H S, Cherikh W S, Chambers D C, Garcia V C, Hachem R R, Kreisel D, Puri V, Kozower B D, Byers D E, Witt C A, Alexander-Brett J, Aguilar P R, Tague L K, Furuya Y, Patterson G A, Trulock E P, 3rd, Yusen R D. Bronchiolitis obliterans syndrome-free survival after lung transplantation: An International Society for Heart and Lung Transplantation Thoracic Transplant Registry analysis. J Heart Lung Transplant 2019; 38: 5-16.
3. Khush K K, Potena L, Cherikh W S, Chambers D C, Harhay M O, Hayes D, Jr., Hsich E, Sadavarte A, Singh T P, Zuckermann A, Stehlik J, International Society for H, Lung T. The International Thoracic Organ Transplant Registry of the International Society for Heart and Lung Transplantation: 37th adult heart transplantation report-2020; focus on deceased donor characteristics. J Heart Lung Transplant 2020; 39: 1003-1015.
4. Glanville A R, Aboyoun C L, Havryk A, Plit M, Rainer S, Malouf M A. Severity of lymphocytic bronchiolitis predicts long-term outcome after lung transplantation. Am J Respir Crit Care Med 2008; 177: 1033-1040.
5. Greenland J R, Jones K D, Hays S R, Golden J A, Urisman A, Jewell N P, Caughey G H, Trivedi N N. Association of large-airway lymphocytic bronchitis with bronchiolitis obliterans syndrome. Am J Respir Crit Care Med 2013; 187: 417-423.
6. Rademacher J, Suhling H, Greer M, Haverich A, Welte T, Warnecke G, Gottlieb J. Safety and efficacy of outpatient bronchoscopy in lung transplant recipients—a single centre analysis of 3,197 procedures. Transplant Res 2014; 3: 11.
7. Chambers D C, Hodge S, Hodge G, Yerkovich S T, Kermeen F D, Reynolds P, Holmes M, Hopkins P M. A novel approach to the assessment of lymphocytic bronchiolitis after lung transplantation—transbronchial brush. J Heart Lung Transplant 2011; 30: 544-551.
8. Dugger D T, Fung M, Hays S R, Singer J P, Kleinhenz M E, Leard L E, Golden J A, Shah R J, Lee J S, Deiter F, Greenland N Y, Jones K D, Langelier C R, Greenland J R. Chronic lung allograft dysfunction small airways reveal a lymphocytic inflammation gene signature. Am J Transplant 2021; 21: 362-371.
9. Iasella C J, Hoji A, Popescu I, Wei J, Snyder M E, Zhang Y, Xu W, Iouchmanov V, Koshy R, Brown M, Fung M, Langelier C, Lendermon E A, Dugger D, Shah R, Lee J, Johnson B, Golden J, Leard L E, Ellen Kleinhenz M, Kilaru S, Hays S R, Singer J P, Sanchez P G, Morrell M R, Pilewski J M, Greenland J R, Chen K, McDyer J F. Type-1 immunity and endogenous immune regulators predominate in the airway transcriptome during chronic lung allograft dysfunction. Am J Transplant 2021; 21: 2145-2160.
10. Tissot A, Danger R, Claustre J, Magnan A, Brouard S. Early Identification of Chronic Lung Allograft Dysfunction: The Need of Biomarkers. Front Immunol 2019; 10: 1681.
11. Orenbuch R, Filip I, Comito D, Shaman J, Pe'er I, Rabadan R. arcasHLA: high-resolution HLA typing from RNAseq. Bioinformatics 2020; 36: 33-40.
12. Greenland J R, Wang P, Brotman J J, Ahuja R, Chong T A, Kleinhenz M E, Leard L E, Golden J A, Hays S R, Kukreja J, Singer J P, Rajalingam R, Jones K, Laszik Z G, Trivedi N N, Greenland N Y, Blanc P D. Gene signatures common to allograft rejection are associated with lymphocytic bronchitis. Clin Transplant 2019; 33: e13515.
13. Reeve J, Einecke G, Mengel M, Sis B, Kayser N, Kaplan B, Halloran P F. Diagnosing rejection in renal transplants: a comparison of molecular- and histopathology-based approaches. Am J Transplant 2009; 9: 1802-1810.
14. Halloran P F, Venner J M, Madill-Thomsen K S, Einecke G, Parkes M D, Hidalgo L G, Famulski K S. Review: The transcripts associated with organ allograft rejection. Am J Transplant 2018; 18: 785-795.
15. Khatri P, Roedder S, Kimura N, De Vusser K, Morgan A A, Gong Y, Fischbein M P, Robbins R C, Naesens M, Butte A J, Sarwal M M. A common rejection module (CRM) for acute rejection across multiple organs identifies novel therapeutics for organ transplantation. J Exp Med 2013; 210: 2205-2221.
16. Khatri A, Todd J L, Kelly F L, Nagler A, Ji Z, Jain V, Gregory S G, Weinhold K J, Palmer S M. JAK-STAT activation contributes to cytotoxic T cell-mediated basal cell death in human chronic lung allograft dysfunction. JCI Insight 2023; 8.
17. Shino M Y, Todd J L, Neely M L, Kirchner J, Frankel C W, Snyder L D, Pavlisko E N, Fishbein G A, Schaenman J M, Mason K, Kesler K, Martinu T, Singer L G, Tsuang W, Budev M, Shah P D, Reynolds J M, Williams N, Robien M A, Palmer S M, Weigt S S, Belperio J A. Plasma CXCL9 and CXCL10 at allograft injury predict chronic lung allograft dysfunction. Am J Transplant 2022; 22: 2169-2179.
18. Verleden S E, Hendriks J M H, Lauwers P, Yogeswaran S K, Verplancke V, Kwakkel-Van-Erp J M. Biomarkers for Chronic Lung Allograft Dysfunction: Ready for Prime Time?Transplantation 2023; 107: 341-350.
19. Parkes M D, Halloran K, Hirji A, Pon S, Weinkauf J, Timofte I L, Snell G I, Westall G P, Havlin J, Lischke R, Zajacova A, Hachem R, Kreisel D, Levine D, Kubisa B, Piotrowska M, Juvet S, Keshavjee S, Jaksch P, Klepetko W, Halloran P F. Transcripts associated with chronic lung allograft dysfunction in transbronchial biopsies of lung transplants. Am J Transplant 2022; 22: 1054-1072.
20. Verleden S E, Vos R, Vanaudenaerde B M, Verleden G M. Chronic lung allograft dysfunction phenotypes and treatment. J Thorac Dis 2017; 9: 2650-2659.
21. Hannan S J, Iasella C J, Sutton R M, Popescu I D, Koshy R, Burke R, Chen X, Zhang Y, Pilewski J M, Hage C A, Sanchez P G, Im A, Farah R, Alder J K, McDyer J F. Lung transplant recipients with telomere-mediated pulmonary fibrosis have increased risk for hematologic complications. Am J Transplant 2023.
22. Wang P, Leung J, Lam A, Lee S, Calabrese D R, Hays S R, Golden J A, Kukreja J, Singer J P, Wolters P J, Tang Q, Greenland J R. Lung transplant recipients with idiopathic pulmonary fibrosis have impaired alloreactive immune responses. J Heart Lung Transplant 2022; 41: 641-653.
23. Furukawa M, Chan E G, Ryan J P, Hyzny E J, Sacha L M, Coster J N, Pilewski J M, Lendermon E A, Kilaru S D, McDyer J F, Sanchez P G. Induction Strategies in Lung Transplantation: Alemtuzumab vs. Basiliximab a Single-Center Experience. Front Immunol 2022; 13: 864545.
24. Ivulich S, Paraskeva M, Paul E, Kirkpatrick C, Dooley M, Snell G. Rescue Everolimus Post Lung Transplantation is Not Associated With an Increased Incidence of CLAD or CLAD-Related Mortality. Transpl Int 2023; 36: 10581.
25. Kulkari H S, Cherikh W S, Chambers D C, et al.: Bronchiolitis obliterans syndrome-free survival after lung transplantation: An International Society for Heart and Lung Transplantation Thoracic Transplant Registry analysis. J Heart Lung Transplant 2019; 38:5-16.
26. Venado A, Kukreja J, Greenland J R: Chronic Lung Allograft Dysfunction. Thorac Surg Clin 2022; 32:231-42.
27. Verleden G M, Raghu G, Meyer K C, Glanville A R, Corns P: A new classification system for chronic lung allograft dysfunction. J Heart Lung Transplant 2014; 33:127-33.
28. Verleden S E, Hendriks J M H, Lauwers P, Yogeswaran S K, Verplancke V, Kwakkel-Van-Erp J M: Biomarkers for Chronic Lung Allograft Dysfunction: Ready for Prime Time?Transplantation 2023; 107:341-50.
29. Dugger D T, Fung M, Hays S R, et al.: Chronic lung allograft dysfunction small airways reveal a lymphocytic inflammation gene signature. Am J Transplant 2021; 21:362-71.
30. Mohanty R P, Moghbeli K, Singer J P, et al.: Small airway brush gene expression predicts chronic lung allograft dysfunction and mortality. J Heart Lung Transplant 2024.
31. Verleden G M, Glanville A R, Lease E D, et al.: Chronic lung allograft dysfunction: Definition, diagnostic criteria, and approaches to treatment-A consensus report from the Pulmonary Council of the ISHLT. J Heart Lung Transplant 2019; 38:493-503.
32. Zuo W L, Rostami M R, Shenoy S A, et al.: Cell-specific expression of lung disease risk-related genes in the human small airway epithelium. Respir Res 2020; 21:200.
33. Yu G, He Q Y: ReactomePA: an R/Bioconductor package for reactome pathway analysis and visualization. Mol Biosyst 2016; 12:477-9.
34. Greenland J R, Jewell N P, Gottschall M, et al.: Bronchoalveolar lavage cell immunophenotyping facilitates diagnosis of lung allograft rejection. Am J Transplant 2014; 14:831-40.
35. Delorey T_{M}, Ziegler C G K, Heimberg G, et al.: COVID-19 tissue atlases reveal SARS-CoV-2 pathology and cellular targets. Nature 2021; 595:107-13.
36. Moshkelgosha S, Duong A, Wilson G, et al.: Interferon-stimulated and metallothionein-expressing macrophages are associated with acute and chronic allograft dysfunction after lung transplantation. J Heart Lung Transplant 2022; 41:1556-69.
37. Calabrese D R, Ekstrand C A, Yellamilli S, et al.: Macrophage and CD8 T cell discordance are associated with acute lung allograft dysfunction progression. J Heart Lung Transplant 2024; 43:1074-86.
38. Bazemore K, Rohly M, Permpalung N, et al.: Donor derived cell free DNA % is elevated with pathogens that are risk factors for acute and chronic lung allograft injury. J Heart Lung Transplant 2021; 40:1454-62.
39. Shino M Y, Todd J L, Neely M L, et al.: Plasma CXCL9 and CXCL10 at allograft injury predict chronic lung allograft dysfunction. Am J Transplant 2022; 22:2169-79.
40. Iasella C J, Hoji A, Popescu I, et al.: Type-1 immunity and endogenous immune regulators predominate in the airway transcriptome during chronic lung allograft dysfunction. Am J Transplant 2021; 21:2145-60.
41. Khatri A, Todd J L, Kelly F L, et al.: JAK-STAT activation contributes to cytotoxic T cell-mediated basal cell death in human chronic lung allograft dysfunction. JCI Insight 2023; 8.
42. Wong A, Duong A, Wilson G, et al.: Ischemia-reperfusion responses in human lung transplants at the single-cell resolution. Am J Transplant 2024.
43. Verleden S E, Vasilescu D M, McDonough J E, et al.: Linking clinical phenotypes of chronic lung allograft dysfunction to changes in lung structure. Eur Respir J 2015; 46:1430-9.
44. Chambers D C, Hodge S, Hodge G, et al.: A novel approach to the assessment of lymphocytic bronchiolitis after lung transplantation—transbronchial brush. J Heart Lung Transplant 2011; 30:544-51.
45. Kang T G, Kwon K W, Kim K, et al.: Viral coinfection promotes tuberculosis immunopathogenesis by type I IFN signaling-dependent impediment of Th1 cell pulmonary influx. Nat Commun 2022; 13:3155.
46. Li F, Zhang R, Li S, Liu J: IDOL: An important immunotherapy target in cancer treatment. Int Immunopharmacol 2017; 47:70-7.
47. Tsao T, Qiu L, Bharti R, et al.: CD94(+) natural killer cells potentiate pulmonary ischaemia-reperfusion injury. Eur Respir J 2024; 64.
48. Su H, Na N, Zhang X, Zhao Y: The biological function and significance of CD74 in immune diseases. Inflamm Res 2017; 66:209-16.
49. Parkes M D, Halloran K, Hirji A, et al.: Transcripts associated with chronic lung allograft dysfunction in transbronchial biopsies of lung transplants. Am J Transplant 2022; 22:1054-72.
50. Mohanty R P, Moghbeli K, Singer J P, et al. Small airway brush gene expression predicts chronic lung allograft dysfunction and mortality. The Journal of Heart and Lung Transplantation. doi:10.1016/j.healun.2024.07.010
51. Martinu T, Todd J L, Gelman A E, Guerra S, Palmer S M. Club Cell Secretory Protein in Lung Disease: Emerging Concepts and Potential Therapeutics. Annu Rev Med. Jan. 27 2023; 74:427-441. doi:10.1146/annurev-med-042921-123443.

1. A method of treating or preventing chronic lung allograft dysfunction (CLAD) in a subject having or at risk of developing CLAD comprising: administering a CLAD therapeutic to the subject identified in need thereof, wherein the subject was identified as being in need thereof by determining that the subject has an Airway Inflammation 2 (AI2) metagene score from a sample obtained from the subject which is higher than a reference AI2 metagene score obtained from a reference population.

2. The method of claim 1, wherein the AI2 metagene score comprises one or more of an RS1 subscore, RS2 subscore, or RS3 subscore.

3. The method of claim 1, wherein the AI2 metagene score comprises expression data from an AI2 metagene comprising the genes MYH9, SAT1, TPM4, MDK, UBD, CD74, HLA-A, HLA-E, HLA-C, HLA-B, IRF1, PSMB9, PSMB8, ISG20, MIDN, APOL3, CXCL11, CXCL10, CXCL9, GBP4, NLRC5, IDO1, TAP1, HLA-F, SERPINA3, ADAMDEC1, CXCL13, INPP5D, KLRD1, FCAR, NKG7, and ADORA2A or the genes listed in Table 1b or the genes listed in Table 1c.

4. The method of claim 3, wherein the AI2 metagene score is determined by:
a) normalizing the number of copies of each gene in the AI2 metagene by variance stabilizing transformation to obtain a subject's normalized gene count for each gene in the AI2 metagene;
b) calculating a subject's total normalized gene count by adding the subject's normalized gene counts for each gene in the AI2 metagene;
c) comparing the subject's total normalized gene count to a reference population data set of total normalized gene counts for each gene in the AI2 metagene, wherein the reference population data set of total normalized gene counts has a reference population mean and a reference population standard deviation; wherein said comparing comprises:
i) subtracting the reference population mean from the subject's total normalized gene count to obtain a numerator; and
ii) dividing the numerator by the reference population standard deviation;
thereby determining the AI2 metagene score.

5. The method of claim 1, further comprising determining an RS1 subscore, RS2 subscore and/or RS3 subscore.

6. The method of claim 5, wherein
a) the RS1 subscore comprises expression data of an RS1 gene cluster comprising the genes MYH9, SAT1, TPM4, MDK, UBD, CD74, HLA-A, HLA-E, HLA-C, HLA-B, IRF1, PSMB9, and PSMB8;
b) the RS2 subscore comprises expression data of an RS2 gene cluster comprising the genes ISG20, MIDN, APOL3, CXCL11, CXCL10, CXCL9, GBP4, NLRC5, IDO1, TAP1, and HLA-F; and
c) the RS3 subscore comprises expression data of an RS3 gene cluster comprising the genes SERPINA3, ADAMDEC1, CXCL13, INPP5D, KLRD1, FCAR, NKG7, and ADORA2A.

7. The method of claim 6, wherein the RS1 subscore is determined by
a) normalizing the number of copies of each gene in the RS1 gene cluster by variance stabilizing transformation to obtain a subject's normalized gene count for each gene in the RS1 gene cluster;
b) calculating a subject's total normalized gene count by adding the subject's normalized gene counts for each gene in the RS1 gene cluster;
c) comparing the subject's total normalized gene count to a reference population data set of total normalized gene counts for each gene in the RS1 gene cluster, wherein the reference population data set of total normalized gene counts has a reference population mean and a reference population standard deviation; wherein said comparing comprises:
i) subtracting the reference population mean from the subject's total normalized gene count to obtain a numerator; and
ii) dividing the numerator by the reference population standard deviation;
thereby determining the RS1 subscore.

8. The method of claim 6, wherein the RS2 subscore is determined by
a) normalizing the number of copies of each gene in the RS2 gene cluster by variance stabilizing transformation to obtain a normalized gene count for each gene in the RS2 gene cluster
b) calculating a subject's total normalized gene count by adding the subject's normalized gene counts for each gene in the RS2 gene cluster;
c) comparing the subject's total normalized gene count to a reference population data set of total normalized gene counts for each gene in the RS2 gene cluster, wherein the reference population data set of total normalized gene counts has a reference population mean and a reference population standard deviation; wherein said comparing comprises:
i) subtracting the reference population mean from the subject's total normalized gene count to obtain a numerator; and
ii) dividing the numerator by the reference population standard deviation;
thereby determining the RS2 subscore.

9. The method of claim 6, wherein the RS3 subscore is determined by
a) normalizing the number of copies of each gene in the RS3 gene cluster by variance stabilizing transformation to obtain a normalized gene count for each gene in the RS3 gene cluster;
b) calculating a subject's total normalized gene count by adding the subject's normalized gene counts for each gene in the RS3 gene cluster;
c) comparing the subject's total normalized gene count to a reference population data set of total normalized gene counts for each gene in the RS3 gene cluster, wherein the reference population data set of total normalized gene counts has a reference population mean and a reference population standard deviation; wherein said comparing comprises:
i) subtracting the reference population mean from the subject's total normalized gene count to obtain a numerator; and
ii) dividing the numerator by the reference population standard deviation;
thereby determining the RS3 subscore.

10. The method of claim 1, wherein the AI2 metagene score is greater than about 0.43.

11. The method of claim 1, wherein the sample was obtained from small airways, optionally wherein the sample comprises basal, club, secretory, secretory-ciliated, ciliated, ionocyte, mast, lymphocyte, and/or monocyte cells.

12.-14. (canceled)

15. The method of claim 1, wherein the CLAD therapeutic is macrolide antibiotic azithromycin, cyclosporine, tacrolimus, fundoplication for gastroesophageal reflux, montelukast, extracorporeal photopheresis (ECP), aerosolized cyclosporine, cytolytic anti-lymphocyte therapies, thymoglobulin, total lymphoid irradiation (TLI), pirfenidone, everolimus, sirolimus, rapamycin, inhaled rapamycin, macitentan, prednisone, baricitinib, anti-CD94 monoclonal antibody, aztreonam lysine inhalation, tocilizumab, mesenchymal stem cells, regadenoson, belumosudil, immunoglobulin and/or belatacept.

16. The method of claim 15, wherein said step of administering a CLAD therapeutic to a subject identified in need thereof comprises administering a higher dose of said CLAD therapeutic than had been administered prior to treating a subject having CLAD.

17.-174. (canceled)

175. A system for diagnosing lung allograft dysfunction, comprising:
a) a sequencing device configured to obtain gene expression data from an airway brush sample of a lung transplant recipient; and
b) a processor configured to:
i) input the gene expression data for a gene set into a random forest machine learning model, and
ii) classify the lung transplant recipient as having chronic lung allograft dysfunction (CLAD) or acute lung allograft dysfunction (ALAD) based on an output of the random forest machine learning model.

176. The system of claim 175, wherein the sequencing device is configured to perform quantitative PCR, reverse transcription-loop mediated isothermal amplification (LAMP), or digital RNA counting.

177. The system of claim 175, wherein the processor is further configured to:
a) obtain gene expression data for cell type-specific genes; and
b) input the gene expression data for the cell type-specific genes into the random forest machine learning model along with the gene set data.

178. The system of claim 177, wherein the cell type-specific genes include genes associated with epithelial subtypes and leukocytes.

179. The system of claim 178, wherein the epithelial subtype genes include one or more of SCGB3A1, MS4A8, KRT5, and CALM1, and the leukocyte genes include one or more of PTPRC, MARCO, and GNLY.

180.-181. (canceled)

182. A non-transitory computer-readable medium storing instructions that, when executed by a processor, cause the processor to perform a method of diagnosing lung allograft dysfunction, the method comprising:
a) receiving gene expression data from an airway brush sample of a lung transplant recipient;
b) inputting the gene expression data for a gene set into a random forest machine learning model; and
c) classifying the lung transplant recipient as having chronic lung allograft dysfunction (CLAD) or acute lung allograft dysfunction (ALAD) based on an output of the random forest machine learning model.

183. The non-transitory computer-readable medium of claim 182, wherein the method further comprises:
a) obtaining gene expression data for cell type-specific genes; and
b) inputting the gene expression data for the cell type-specific genes into the random forest machine learning model along with the gene set data.

184.-187. (canceled)
